(19) United States
(12) Patent Application Publication
(10) Pub. No.: US 2022/0105051 A1
(43) Pub. Date: Apr. 7, 2022

(54) COMPOUNDS FOR INCREASING MHC-I EXPRESSION AND MODULATING HISTONE DEACETYLASE ACTIVITY

(71) Applicant: Cava Healthcare Inc., Surrey (CA)

(72) Inventors: Wilfred Jefferies, Surrey, British Columbia (CA); Samantha Ellis, Surrey, British Columbia (CA); Ray Anderson, Surrey, British Columbia (CA); Sarah Dada, Surrey, British Columbia (CA); Ping Cheng, Surrey, British Columbia (CA); Cheryl Pfeifer, Surrey, British Columbia (CA); David Williams, Surrey, British Columbia (CA); Lilian Nohara, Surrey, British Columbia (CA)

(73) Assignee: Cava Healthcare Inc., Surrey, BC (CA)

(21) Appl. No.: 17/426,860

(22) PCT Filed: Jan. 30, 2020
(86) PCT No.: PCT/CA2020/050112

§ 371 Date: Jul. 29, 2021

Related U.S. Application Data
(60) Provisional application No. 62/799,305, filed on Jan. 31, 2019.

(51) Int. Cl. A61K 31/05; A61P 35/00; A61K 45/06; A61K 31/085

(52) U.S. Cl. CPC A61K 31/05; A61K 31/085; A61K 45/06; A61P 35/00

(57) ABSTRACT

An object of the present invention is to provide a compound for modulating expression of Major Histocompatibility Complex Class I (MHC-1) and/or TAP-1, in eukaryotic cells. In certain aspects, the compound is a curcuphenol, a terpene or a cannabinoid. Also provided are a composition that comprises the compound and methods of use thereof, for instance, for augmenting an immune response involving MHC-1 CTL, treating cancer, or treating a disease associated with histone acetylation abnormalities.

### FIELD OF THE INVENTION

This invention pertains generally to disease therapeutics and in particular, to compounds for increasing MHC-I expression and modulating histone deacetylases activities.

### BACKGROUND OF THE INVENTION

Cancer is a devastating disease that arises from genetic and epigenetic modifications. A common signature across several forms of cancer, particularly the deadliest form, metastatic, is loss of immunogenicity and consequently, immune evasion. This can be achieved through several mechanisms, one of which involves loss of the antigen presentation machinery (APM). A key component to APM are the Major histocompatibility complexes.

Major histocompatibility complex class I (MHC-I) antigens are found on nearly all nucleated cells of the body. The primary function of this class of major histocompatibility complex (MHC) molecules is to display (or present) peptide fragments of intracellular proteins to cytotoxic T lymphocytes (CTLs). Based on this display, CTLs will ignore healthy cells and attack those displaying MHC-bound foreign or otherwise abnormal peptides, including disease-associated peptide (antigens) such as cancer antigens. Thus, the surface expression of MHC-I molecules plays a crucial role in determining the susceptibility of target cells to CTLs.

Many cancerous cells display down-regulated MHC-I cell surface expression (see, for example, Jefferies et al, J Immunol Sep. 15, 1993, 151 (6) 2974-2985); Gabathuler et al., J Exp Med (1994) 180 (4): 1415-1425.; Alimonti et al., Nature Biotechnology 18: 515-520(2000); Wang et al., JBC. 283: 3951-3959, 2008; Chang et al., Keio J. Med. 52:220-9, 2003; Zagzag et al., Lab Invest. 85:328-41, 2005; and Hewitt, Immunology. 110:163-69, 2003). Reduced MHC-I expression can result at least in part from the down-regulation of multiple factors such as transporters (for example, TAP-1, TAP-2), proteasome components (LMP), and other accessory proteins involved in the antigen presentation and processing pathway. This characteristic may allow cancerous cells to evade immune surveillance and thereby provide a survival advantage against immune activity otherwise designed to eliminate the cells.

Accordingly, there is a need in the art for agents that can increase MHC class I expression in these and other types of diseased cells and thereby improve the ability of the immune system to target such cells for destruction.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide compounds for increasing MHC-I expression and modulating histone deacetylases activity.

In one aspect of the present invention, there is provided a compound which modulates expression of MHC-1 and/or TAP-1, in eukaryotic cells. In certain aspects, the compound is a terpene. In certain aspects, the compound is a curcuphenol. In certain aspects, the compound is a cannabinoid.

In one aspect of the present invention, there is provided a compound which modulates expression of MHC-1 and/or TAP-1, in eukaryotic cells and having the structure:

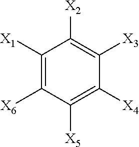

where:

X₁ is H, R, OH, OR, SH, SR, F, Cl, Br, I, OCOR, NH₂, RNH, R₂NH, NHCOR, OSO₃H, OP(OH)₃

X₂ is R₁

X₃ is H, R, OH, OR, SH, SR, F, Cl, Br, I, OCOR, NH₂, RNH, R₂NH, NHCOR, OSO₃H, OP(OH)₃

X₄ and X₆ are independently H, R, OH, OR, SH, SR, F, Cl, Br, I, OCOR, NH₂, RNH, R₂NH, NHCOR, OSO₃H, OP(OH)₃

X₅ is R₂

R is a linear, branched, or cyclic, saturated or unsaturated, one to thirty carbon alkyl group that may be substituted with one or more of OH, OR, SH, SR, ═O, F, Cl, Br, I, OCOR, NH₂, RNH, R₂NH, NHCOR, OSO₃H, OP(OH)₃, and where individual carbon atoms may be replaced by O, N, or S atoms.

R₁ is a linear, branched, or cyclic, saturated, unsaturated or aromatic, one to thirty carbon alkyl group that may be substituted with one or more of OH, OR, SH, SR, ═O, F, Cl, Br, I, OCOR, NH₂, RNH, R₂NH, NHCOR, OSO₃H, OP(OH)₃, and where individual carbon atoms may be replaced by O, N, or S atoms.

R₂ is a linear, branched, or cyclic, saturated, unsaturated, or aromatic one to twenty carbon alkyl group that may be substituted with one or more of OH, OR, SH, SR, ═O, F, Cl, Br, I, OCOR, NH₂, RNH, R₂NH, NHCOR, OSO₃H, OP(OH)₃, and where individual carbon atoms may be replaced by O, N, or S atoms.

In specific aspects, the compounds of the invention modulate HDAC activity as compared to activity untreated control cells.

In specific aspects, the compounds of the invention inhibits HDAC8 activity and upregulates HDAC5 and HDAC10.

In specific aspects, X₁ is OH or OR; X₂ is one of the following:

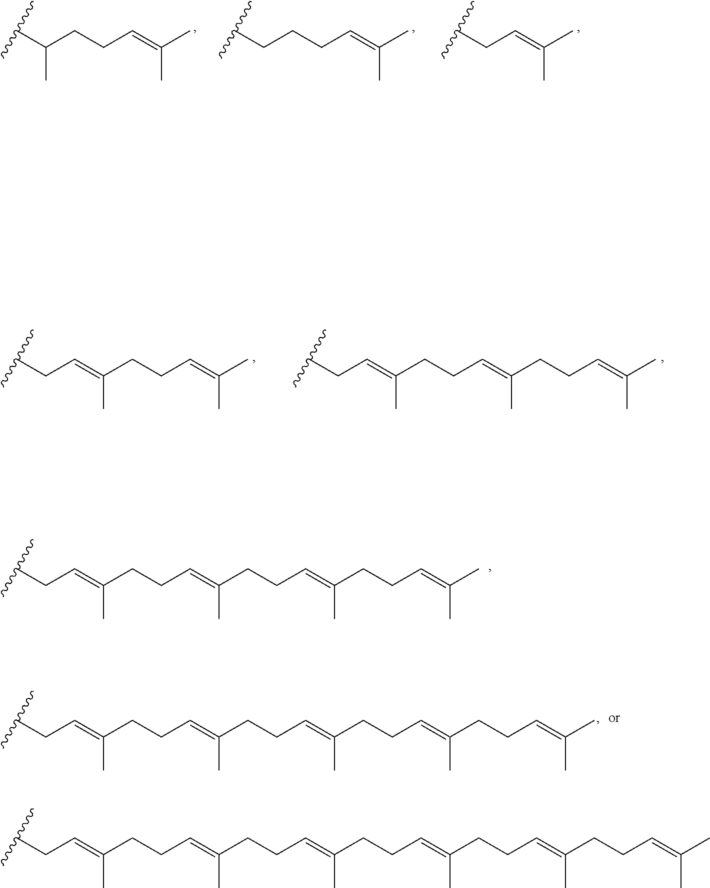

X₃ is H, OH, or OR; X₄ and X₆ is H; X₅ is OH, OR, or methyl, ethyl, n-propyl, n-butyl, n-pentyl, n-hexyl or any seven to twenty carbon linear saturated n-alkyl

In specific aspects, the compounds of the invention have structure:

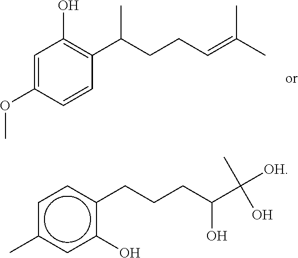

In another aspect of the present invention, there is provided a method of treating cancer comprising administering one or more compounds of the invention alone or in combination with one or more other therapeutic agents.

In another aspect of the present invention, there is provided method of modulating histone acetylation comprising administering one or more compounds of the invention alone or in combination with one or more other therapeutic agents.

In another aspect of the present invention, there is provided a method of treating a disease associated with histone acetylation abnormalities comprising administering one or more compounds of the invention or in combination with one or more other therapeutic agents. Optionally, the disease is selected from cancer, a mood disorder or epilepsy.

In another aspect of the present invention, there is provided a method of augmenting an immune response, improving general health, improving longevity and/or reducing nausea comprising administering one or more compounds of the invention alone or in combination with one or more other therapeutic agents.

In another aspect of the present invention, there is provided a method of augmenting an immune response involving MHC-1 CTL comprising administering one or more compounds of the invention alone or in combination with one or more other therapeutic agents. For example, an immune response to viruses, bacteria and/or fungus. Exemplary viruses include but are not limited to herpes viruses.

In another aspect of the present invention, there is provided a composition comprising one or more compounds of the invention alone or in combination with one or more other therapeutic agents and a carrier. Optionally, the composition comprises a compound having the structure:

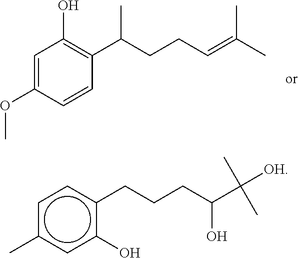

### BRIEF DESCRIPTION OF THE DRAWINGS

These and other features of the invention will become more apparent in the following detailed description in which reference is made to the appended drawings.

FIG. 1 shows endogenous antigen presentation pathway. The pathway though which endogenous proteins are processed and presented to cytotoxic T lymphocytes (CD8^{+/+}) cells of the immune system via the major histocompatibility complex I molecules.

FIG. 2 shows characterization of antigen presentation machinery proteins, TAP-1 and MHC-I, in TC-1 and antecedent A9 cell lines in vitro. (A) Levels of TAP-1 protein measured by Western blot in TC-1 and A9 cell lines. (B) Surface expression levels of MHC-I (PE-A) on TC-1 (blue) and A9 (red) cell lines measured by flow cytometry.

FIG. 3 shows characterization of immune response to the TC-1 cell line in vivo. To examine the immunological characteristics of the TC-1 cell line in vivo 5×10⁵ cells were subcutaneously injected into the right flank of 32 mice: C57BL/6 (n=8), GATA1^{-/-} (n=8), CD4^{-/-} (n=8), and CD8^{-/-} (n=8). (A) Body weight was recorded three times a week until humane end point. (B) Tumour volume was measured three times a week (V=L×W²). (C) After 34 days all mice were euthanized and tumour weights were measured. Outliers were removed if two SEM outside the average calculated for each group.

FIG. 4 shows immune response to A9 cell line in vivo. To examine the immunological characteristics of the A9 cell line in vivo 5×10⁵ cells were subcutaneously injected into the right flank of 32 female mice: C57BL/6 (n=8), GATA1^{-/-} (n=8), CD4^{-/-} (n=8), and CD8^{-/-} (n=8). (A) Body weight was recorded three times a week until humane end point. (B) Tumour volume was measured three times a week (V=L×W²). (C) After 14 days all mice were euthanized and tumour weights were measured. Outliers were removed if two SEM outside the average calculated for each group.

FIG. 5 shows screening of two generations of curcuphenol analogues for induction of MHC-I on the cell surface of A9 cell line in vitro. (A) Cells were plated (Day 0) at a density of 10⁵ cells/well in a 6 well plate. After 24 hours they were treated with one of curcuphenol analogues at a range of concentrations (0.0067 mg/mL, 0.02 mg/mL, or 0/06 mg/mL). After 48 hours the cells were analyzed by flow cytometry expression of MHC-I at the cell surface. (B) Structure of P02-113 and P03-97-1.

FIG. 6 shows pharmacokinetic analyses of P02-113 and P03-97-1. Female C57BL/6 mice, between the ages of 6-8 weeks, were i.p. injected with 5.2 mg/kg of P02-113 or P03-97-1 and blood was collected by cardiac puncture from mice at various time points (n=3) following injection. Plasma was isolated from blood and shipped on dry ice, to TMIC for PK analysis.

FIG. 7 shows in vivo analyses of anti-cancer effects of P02-113 and P03-97-1. Thirty-two C57BL/6 mice were injected subcutaneously in the right flank by i.p. with 5×10⁴ A9 cells. After seven days mice were randomized into four treatment groups (8 mice per group): vehicle (1% DMSO), TSA (0.5 mg/kg, positive control), P02-113 (5.2 mg/kg), or P03-97-1 (5.2 mg/kg), and were treated daily for twelve days. Body weights of mice (A) and tumour volumes (B) were calculated (V=L×W²) three times a week. Following 12 days of treatment mice were euthanized and tumours were removed and weighed (C).

FIG. 8 shows analysis of T cell infiltration of tumours in vivo. C57BL/6 mice were injected with 5×10⁴ A9 cells, subcutaneously in the right flank. Seven days after injection mice were divided into four treatment groups: vehicle (a), TSA (0.5 mg/kg) (b), P02-113 (5.2 mg/kg)(c), or P03-97-1 (5.2 mg/kg)(d). Following 12 days of treatment tumours were removed and analyzed by flow cytometry for anti-CD4+ (APC) and anti-CD8+ (PE-Cy7) infiltration.

FIG. 9 shows class I/II histone deacetylase assay measuring HDAC activity in A9 cells after treatment with P02-113 or P03-97-1. The HDAC-Glo™ I/II Assay and Screening System (Promega) was used to measure the activities of P02-113 and P03-97-1 on the class I/II HDACs in the A9 cells in vitro. The linear range of the A9 cells was first determined following the assay protocol After optimization of A9 cell density the cells were plated at a concentration of 30,000 cells/ml and left overnight at 37° Celsius. The cells were then treated with vehicle, TSA (50 nM), or a range of concentrations of P02-113 or P03-97-1. After completing the assay following the screening protocol the fluorescence was measured using the Infinite M200 (Tecan) with i-control software (Tecan).

FIG. 10 shows class I HDAC enzymes unaffected by P02-113 or P03-97-1. The Class I HDACs were evaluated for activity after treatment with P02-113 or P03-97-1 using respective HDAC Fluorogenic kits (BPS Biosciences). HDAC1-3 showed no change in activity upon treatment with either P02-113 or P03-97-1 at concentrations ranging from 5 μm to 0.02 μm.

FIG. 11 shows HDAC8, a class I HDAC, showed a change in activity when exposed to P02-113 or P03-97-1. HDAC8 was the only HDAC that showed slight inhibition at lower concentrations for both compounds.

FIG. 12 shows HDAC class II Fluorogenic assay of HDACs unaffected by P02-113 or P03-97-1. HDACs 4,6,7 and 9 remains unaffected by analogues at concentrations, 5 μm to 0.02 μm, tested.

FIG. 13 shows class II HDAC assay of HDACs with enhanced activity upon treatment with either P02-113 or P03-97-1. HDAC 5 and 10 were the only class II HDACs showing an increase in activity levels upon treatment with curcuphenol analogues. Enhancement of HDAC activity is novel among the class 1, 11 and IV enzymes. HDAC10 was enhanced at all concentrations tested, while HDAC5 showed limitations between the concentrations of 0.02-2.5 μM, for both compounds.

FIG. 14 shows analysis of activity of SIRT1, from the class III HDAC family, after treatment with P02-113 or P03-97-1. SIRT1 showed no change in activity upon treatment with compounds P02-113 or P03-7-1 between the concentrations of 5 μm to 0.02 μm. Activity was measure using the SIRT1 HDAC Fluorogenic kits in which nicotinamide was provided as the positive control as an inhibitor (BPS Biosciences).

FIG. 15 shows class IV HDAC activity (HDAC11) was unaffected after treatment with P02-113 or P03-97-1. Activity of HDAC11 was measured using the HDAC-Glo™ I/II Assay and Screening System (Promega) and HDAC11 (BPS Biosciences) at a concentration of 60 ng/mL. HDAC11 showed no change in activity upon treatment with P02-113 or P03-97-1 between the concentrations 5 μm to 0.02 μm.

FIG. 16 shows the effect of the Curcuphenol on A9 cells treated for 48 hours at concentrations of 0.032 μmol, 0.064 μmol, and 0.128 μmol. MHC class I upregulation was found to be upregulated upon curcuphenol treatment relative to DMSO treated cells. Upon treatment with 0.128 μmol of Curcuphenol, live cell frequency drops substantially.

FIG. 17 shows the effect of the Curcuphenol on A9 cells treated for 48 hours at concentrations of 0.055 umol, 0.064 umol, and 0.071 umol. MHC class I upregulation was found to be upregulated upon curcuphenol treatment relative to DMSO treated cells. Upon treatment with 0.128 umol of Curcuphenol, live cell frequency drops substantially. Optimum MHC upregulation and live cell frequency is at 0.064 umol.

FIG. 18 shows the treatment with Curcuphenol at 0.064 μmol causes increased mRNA expression of TAP, MHC class I, and HDACs 8 and 10.

FIG. 19 shows curcuphenol causes a change in cell growth and differentiation cytokine profile in A9 cells, relative to DMSO treated cells. Red (circles) denotes 0.064 μmol Curcuphenol-treated fold change, and black (triangles) denotes IFN gamma treated A9 cell fold change.

FIG. 20 shows curcuphenol causes a change in inflammation cytokine profile in A9 cells, relative to DMSO treated cells. Red (circles) denotes 0.064 μmol Curcuphenol-treated fold change, and black (triangles) denotes IFN gamma treated A9 cell fold change.

FIG. 21 shows curcuphenol causes a change in leukocyte migration cytokine profile in A9 cells, relative to DMSO treated cells. Red (circles) denotes 0.064 μmol Curcuphenol-treated fold change, and black (triangles) denotes IFN gamma treated A9 cell fold change.

FIG. 22 shows curcuphenol causes a change in inflammation cytokine profile in A9 cells, relative to DMSO treated cells. Cytokines are related to Angiogenesis, immune regulation, leukocyte development, and metabolism. Circles denotes 0.064 μmol Curcuphenol-treated fold change, and Triangles denotes IFN gamma treated A9 cell fold change.

FIG. 23 shows curcuphenol causes a change in cytokine profile in A9 cells, relative to DMSO treated cells. Red (circles) denotes 0.064 μmol Curcuphenol-treated fold change, and black triangles denotes IFN gamma treated A9 cell fold change.

FIG. 24 shows high-throughput screen to identify compounds that are able to induce expression of TAP-1. A. Image acquisition, segmentation and analysis of 96-well plates were carried out using the Cellomics™ Arrayscan VTI automated fluorescence imager. Images of the DNA staining and TAP promoter-induced GFP expression are shown. Segmentation to delineate the nuclei based on the DNA staining fluorescence intensity was performed to identify individual objects and create a cytoplasmic mask around the nuclei in which total GFP fluorescence is measured. Average GFP fluorescence intensity (intensity per cell per pixel) and total number of cells per well were determined. B. IFN-γ treatment induces high level of GFP expression in TAP-deficient cancer cells. LMD:TAP-1 cells were treated with 10 ng/mL of IFN-γ or 1% DMSO vehicle control. Images were taken by Cellomics ArrayScan VTI with the same exposure time. Lines indicate the average GFP intensities.

FIG. 25 shows a summary of high-throughput screen to identify marine extracts able to induce APM in metastatic cells. A. Results from high-throughput screen of 480 marine invertebrate extracts looking at TAP-1 expression in LMD:TAP-1 cell line. Extracts with greater then 40% activity for TAP-1 and within 1 SD of the DMSO negative control were selected as candidates for further analysis (red dots). B. Table summarizing activity and viability of seven extracts that were selected for further analysis after initial high-throughput screen.

FIG. 26 shows identification of two selected marine extracts with the ability to induce MHC-I in metastatic cells. A. Two of the selected extracts, 2 (76018) and 5 (76336), had highly replicable TAP-1 activity at varying concentrations in the LMD:TAP-1 cell line that was measured using the high-throughput screen. MHC-1 expression was quantified using flow cytometry with extracts 2 and 5 at varying concentrations in the A9 cell line. B. Extracts 2 and 5 were fractionated to identify the components inducing the expression of MHC-I. The fractionated compounds were tested for their ability to induce MHC-I in the A9 cell lines, 48 hours after treatment using flow cytometry.

FIG. 27 shows structure of curcuphenol and curcuphenol analogues. A. Structure of the active component in extract 2 (76018), curcuphenol, as well as the two synthesized analogues, P02-113 and P03-97-1 that resulted in the highest expression of MHC-I and lowest cytotoxicity in the A9 cell line. B. The ability of P02 and P03 curcuphenol analogues to induce MHC-I expression was assessed by flow cytometry.

FIG. 28 shows in vivo treatment with PC-02-113 or P03-97-1 suppresses growth of tumors derived from APM-deficient cells. 4×105 A9 cells were s.c. injected into C57BL/6 syngeneic mice. Seven days after inoculation, mice were i.p. injected with PC-02-113 (5.2 mg/kg), P03-97-1 (5.2 mg/kg), TSA (0.5 mg/kg) or vehicle control (1% DMSO) everyday for 12 days. Body weight (A) and tumour volume (B) were assessed three times per week. Mice that did not develop tumours during the study were removed for the analysis, as outliers. C. Following 12 days of treatment with vehicle (a), TSA (b), P02-113 (c), or P03-97-1 (d), tumours were removed and analyzed by flow cytometry for anti-CD4+ (APC) and anti-CD8+ (PE-Cy7) infiltration.

FIG. 29 shows effects of P02-113 and P03-97-1 on class I/II histone deacetylase activity. A. Class I/II histone deacetylase assay measuring HDAC activity in A9 cells after treatment with P02-113 or P03-97-1. A9 cells were plated at a concentration of 30,000 cells/mL and left overnight at 37° C. The cells were then treated with vehicle, TSA (50 nM), or a range of concentrations of P02-113 or P03-97-1. After completing the assay following the screening protocol, the fluorescence was measured using the Infinite M200 (Tecan) with i-control software (Tecan). B. HDAC8, a class I HDAC, showed a change in activity when exposed to P02-113 or P03-97-1. HDAC8 was the only HDAC that showed slight inhibition at lower concentrations for both compounds. C. Class II HDAC assay of HDACs with enhanced activity upon treatment with either P02-113 or P03-97-1. HDAC 5 and 10 were the only class II HDACs showing an increase in activity levels upon treatment with curcuphenol analogues.

FIG. 30 provides testing overview of isolated extracts. Blue (lighter lettering) denotes compounds which exhibit considerable activity.

FIG. 31 shows structure of curcuphenol analogues of the invention (PC-02-113, PC-03-97-1 and P04-149) compared to known anti-cancer agents: TSA and SAHA and curcuphenol.

FIG. 32 shows surface expression of MHC-I was increased after treatment of lung metastatic cancer cell line (A9) with curcuphenol analogues.

FIG. 33 shows water soluble Curcuphenol analogue, P04-149, increases MHC-I expression in A9 cells.

FIG. 34 illustrates epigenetic changes following treatment with Interferon gamma. Briefly, A9 metastatic lung carcinomas were treated with interferon gamma or control (DMSO) and acetylation levels h3k27ac cistrome epigenetic marks around the genes in the A9 genome were compared. h3k27ac cistrome are transcriptionally active marks.

FIG. 35 provides a functional annotation of lost, gained and common regions identified in FIG. 34.

FIG. 36 illustrates an investigation of dmso/Cannabigerol (cann1)/interferon gamma (ifnr) acetylation levels on gained and lost regions. The ifnr compare gained regions with cann1 regions.

FIG. 37 illustrates Gene Ontology analysis of these regions (top 10)

FIG. 38 illustrates investigation of common regions from (ifnr and dmso) comparison. data shows clustering and unclustered way.

FIG. 39 illustrates cann1 active or non active vs ifnr too active or ifnr some active.

FIG. 40 illustrates an investigation of dmso/curcuphenol (curc1)/interferon gamma (ifnr) acetylation levels on gained and lost regions. (on left). The ifnr compare gained regions with curc1 regions.

FIG. 41 illustrates Gene Ontology analysis of these regions (top 10) FIG. 42 illustrates investigation of common regions from (ifnr and dmso) comparison. data shows clustering and unclustered way.

FIG. 43 illustrates curc1 active or nonactive vs ifnr too active or ifnr some active.

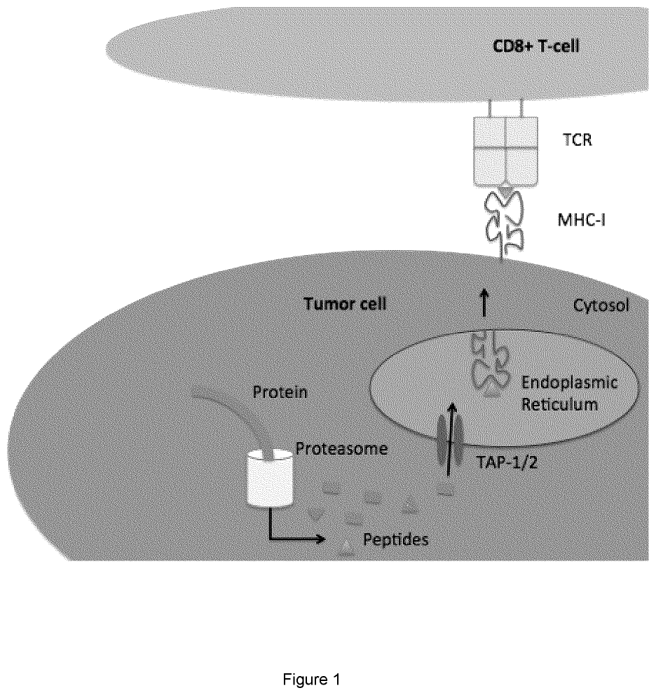

### DETAILED DESCRIPTION OF THE INVENTION

Recognition of MHC-I/peptide complexes is crucial for CTL-mediated immune surveillance of cells. Because certain diseased cells such as cancer cells evade immune surveillance by down-regulating MHC-I cell surface expression, often by down-regulating expression proteins of the antigen presentation pathway such as TAP-1, compounds which restore MHC-I surface expression and presentation of MHC-I/peptide antigen complexes may improve CTL-mediated immune activity towards these diseased cells.

The present invention relates to the discovery that a number of compounds enhance antigen presentation by increasing MHC-I cell surface expression and/or decrease histone deacetylase (HDAC) activity. In certain embodiments, the compounds of the invention increase the expression of TAP-1 (Transporter associated with Antigen Processing 1), a transporter protein of the MHC-I antigen presentation pathway. These compounds may be useful in stimulating an immune response and/or in the treatment of diseases associated with reduced MHC-I surface expression and/or TAP-1 expression, including many cancers.

Compounds

The present invention is directed to compounds that enhance expression of one or more components of the antigen presentation machinery (APM) in cells including but not limited to cells having a reduction in APM, such as certain cancer cells. In certain embodiments, the compounds have the structure:

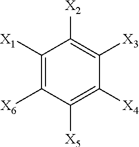

Where:

X₁ is H, R, OH, OR, SH, SR, F, Cl, Br, I, OCOR, NH₂, RNH, R₂NH, NHCOR, OSO₃H, OP(OH)₃

X₂ is R₁

X₃ is H, R, OH, OR, SH, SR, F, Cl, Br, I, OCOR, NH₂, RNH, R₂NH, NHCOR, OSO₃H, OP(OH)₃

X₄ and X₆ are independently H, R, OH, OR, SH, SR, F, Cl, Br, I, OCOR, NH₂, RNH, R₂NH, NHCOR, OSO₃H, OP(OH)₃

X₅ is R₂

R is a linear, branched, or cyclic, saturated or unsaturated, one to thirty carbon alkyl group that may be substituted with one or more of OH, OR, SH, SR, ═O, F, Cl, Br, I, OCOR, NH₂, RNH, R₂NH, NHCOR, OSO₃H, OP(OH)₃, and where individual carbon atoms may be replaced by O, N, or S atoms.

R₁ is a linear, branched, or cyclic, saturated, unsaturated or aromatic, one to thirty carbon alkyl group that may be substituted with one or more of OH, OR, SH, SR, ═O, F, Cl, Br, I, OCOR, NH₂, RNH, R₂NH, NHCOR, OSO₃H, OP(OH)₃, and where individual carbon atoms may be replaced by O, N, or S atoms.

R₂ is a linear, branched, or cyclic, saturated, unsaturated, or aromatic one to twenty carbon alkyl group that may be substituted with one or more of OH, OR, SH, SR, ═O, F, Cl, Br, I, OCOR, NH₂, RNH, R₂NH, NHCOR, OSO₃H, OP(OH)₃, and where individual carbon atoms may be replaced by O, N, or S atoms.

In certain embodiments:

X₁ is OH or OR

X₂ is a linear saturated or unsaturated one to thirty carbon alkyl group containing methyl substituents

X₃ is H, OH, or OR

X₄ and X₆ is H, OH, R1, or OR

X₅ is OH, OR, or R₁

In certain embodiments:

X₁ is OH or OR

X₂ is one of the following:

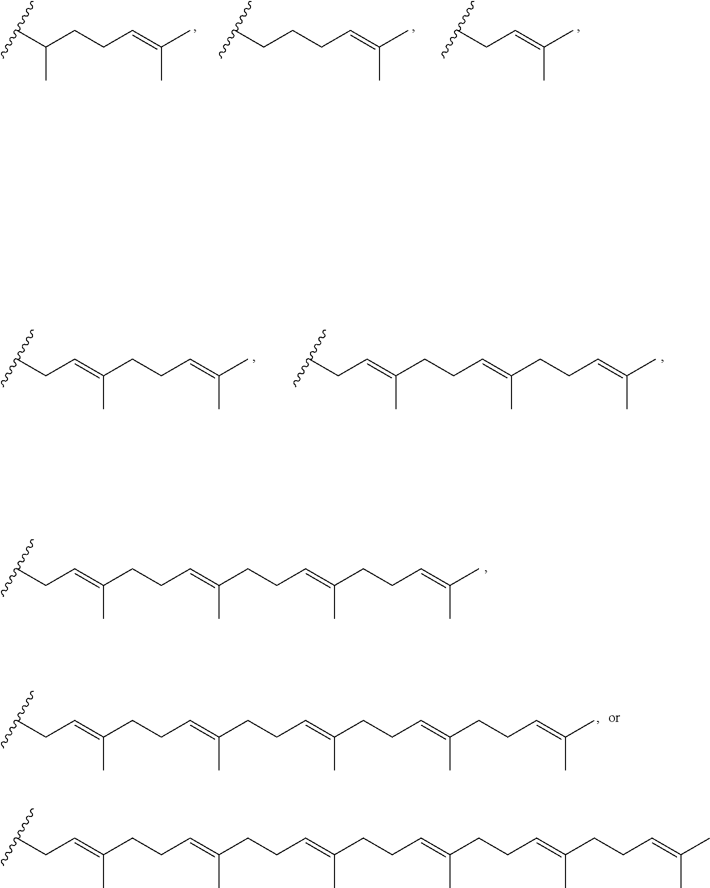

X₃ is H, OH, or OR

X₄ and X₆ is H

X₅ is OH, OR, or methyl, ethyl, n-propyl, n-butyl, n-pentyl, n-hexyl or any seven to twenty carbon linear saturated n-alkyl

Non-limiting examples include:

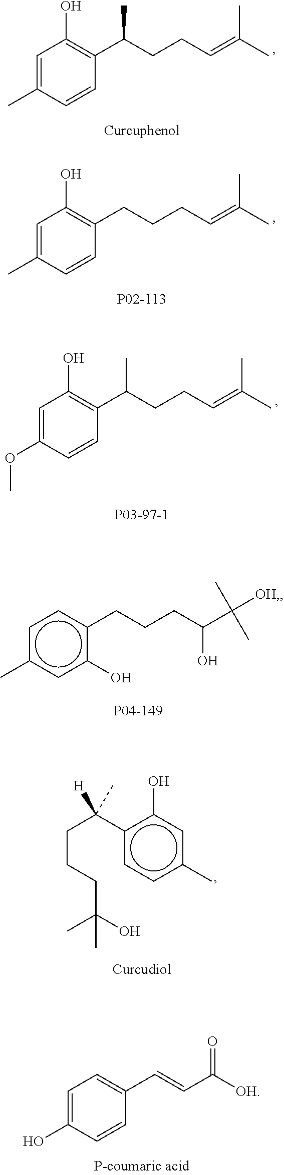

Also provided are enantiomers, stereoisomers, diastereomers, and other stereoisomeric forms, racemates, tautomers, metabolites, and prodrugs of the compounds of the invention. Also included are pharmaceutically acceptable salts of the compounds of the invention, including acid and base addition salts.

In certain embodiments, the compounds are terpenes. In certain embodiments, the compounds are sesquiterpene phenols. In specific embodiments, the compounds are curcuphenol compounds. In certain embodiments, the curcuphenol compounds are water soluble. Non-limiting examples of curcuphenol compounds include but are not limited to Curcuphenol, P02-113, P03-97-1, P04-149, Curcudiol and p-coumaric acid.

In certain embodiments, the compounds are cannabinoids. As used herein, a cannabinoid compound refers to terpenophenolic compounds that binds to a cannabinoid receptor, such as cannabinoid receptor 1 or 2. Generally, there are three types of cannabinoids: phytocannabinoids, endogenous cannabinoids and synthetic cannabinoids. Exemplary cannabinoid compounds include but are not limited to THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin) and CBT (cannabicitran).

In some embodiments, the compound(s) of the present invention are chemically synthesized. Methods of chemical synthesis are known in the art.

In some embodiments, the compounds of the present invention are in natural extracts. In specific embodiments the natural extracts are marine sponge extracts or plant extracts (including but not limited to terrestrial plants). Exemplary genera of plants and sponges include but are not limited to Annona, Abies, Picea, Cedrus, Pinus, Tsuga, Larix, Sciadopitys, Torreya, Cryptomeria, Cannabis, Echinacea, Acmella, Helichrysum, Radula, Piper, Theobroma, Rhododendron, Lepidium, Salvia, Didiscus, Myrmekioderma, Epipolapsis, Pseudopterogorgia, Elvira and Laisanthaea. Exemplary species of these marine sponges and plants include but are not limited to Didiscus flavus, Didiscus oxeata, Myrmekioderma styx, Pseudopterogorgia rigida, Elvira biflora, Laisanthaea podocephala, Glycyrrhiza glabra, Annona squamosa, Annona muricate, Helichrysum umbraculigerum, Radula marginata, Piper nigrum, Piper methusticum, Theobroma cacao, Tuber melanosporum, Rhododendron anthopogonoides, Lepidium meyenii, Salvia Rosmarinus, and Patrinia herterophylla. In certain embodiments, the purity of the compound(s) in the extract is about or at least about 50%, 60%, 70%, 80%, 90%, 95%, 96%, 97%, 98%, 99%, or 100%.

In some embodiments, there is provided resins comprising one or more of the compounds of the invention. Exemplary resins include but is not limited to resins from Pinophyta (also known as Coniferophyta or commonly as conifers).

In some embodiments, the extract comprising one or more of the compounds of the invention is an extract from Tumeric (Curcuma longa), soursop (Annona muricate) or sweetsop (Annona squamosa). In certain embodiments, the extract comprises a curcuminoid. In specific embodiments, the extract comprises curcumin.

In some embodiments, the extract comprising one or more of the compounds of the invention is an extract from Cannabaceae. Exemplary Cannabaceae include but are not limited to Cannabis (e.g. hemp and marijuana) and Humulus (hop).

Pharmaceutical Compositions

The present invention further provides pharmaceutical compositions comprising one or more of the compounds of the present invention, alone or in combination with one or more other agents optionally with a pharmaceutically acceptable carrier, diluent or excipient. As used herein, “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved for use in humans or domestic animals.

Other agents include diagnostic and/or therapeutic agents. Exemplary therapeutic agents include but are not limited to anti-cancer agents and immune stimulatory agents. Examples of anti-cancer agents include small molecules, immunotherapeutics such as vaccines, antibodies, cytokines and cell-based therapies, among others known in the art.

In certain embodiments, one or more compounds of the present invention are used in combination with one or more anti-cancer agents. In specific embodiments, the one or more anti-cancer agents are one or more cytotoxic, chemotherapeutic, immunotherapeutic or anti-angiogenic agents. Particular examples include alkylating agents, anti-metabolites, anthracyclines, anti-tumor antibodies, platinums, type I topoisomerase inhibitors, type II topoisomerase inhibitors, vinca alkaloids, and taxanes.

Non-limiting exemplary small molecules include chlorambucil, cyclophosphamide, cilengitide, lomustine (CCNU), melphalan, procarbazine, thiotepa, carmustine (BCNU), enzastaurin, busulfan, daunorubicin, doxorubicin, gefitinib, erlotinib idarubicin, temozolomide, epirubicin, mitoxantrone, bleomycin, cisplatin, carboplatin, oxaliplatin, camptothecins, irinotecan, topotecan, amsacrine, etoposide, etoposide phosphate, teniposide, temsirolimus, everolimus, vincristine, vinblastine, vinorelbine, vindesine, CT52923, paclitaxel, imatinib, dasatinib, sorafenib, pazopanib, sunitnib, vatalanib, geftinib, erlotinib, AEE-788, dichoroacetate, tamoxifen, fasudil, SB-681323, semaxanib, donepizil, galantamine, memantine, rivastigmine, tacrine, rasigiline, naltrexone, lubiprostone, safinamide, istradefylline, pimavanserin, pitolisant, isradipine, pridopidine (ACR16), tetrabenazine, bexarotene, glatirimer acetate, fingolimod, and mitoxantrone, including pharmaceutically acceptable salts and acids thereof.

Non-limiting exemplary antibodies include 3F8, 8H9, abagovomab, adecatumumab, afutuzumab, alacizumab (pegol), alemtuzumab, altumomab pentetate, amatuximab, anatumomab mafenotox, apolizumab, arcitumomab, bavituximab, bectumomab, belimumab, bevacizumab, bivatuzumab (mertansine), brentuximab vedotin, cantuzumab (mertansine), cantuzumab (ravtansine), capromab (pendetide), carlumab, catumaxomab, cetuximab, citatuzumab (bogatox), cixutumumab, clivatuzumab (tetraxetan), conatumumab, dacetuzumab, daclizumab, dalotuzumab, detumomab, drozitumab, ecromeximab, edrecolomab, elotuzumab, enavatuzumab, ensituximab, epratuzumab, ertumaxomab, etaracizumab, farletuzumab, FBTA05, figitumumab, flanvotumab, galiximab, gemtuzumab, ganitumab, gemtuzumab (ozogamicin), girentuximab, glembatumumab (vedotin), ibritumomab tiuxetan, icrucumab, igovomab, indatuximab ravtansine, intetumumab, inotuzumab ozogamicin, ipilimumab (MDX-101), iratumumab, labetuzumab, lexatumumab, lintuzumab, lorvotuzumab (mertansine), lucatumumab, lumiliximab, mapatumumab, matuzumab, milatuzumab, mitumomab, mogamulizumab, moxetumomab (pasudotox), nacolomab (tafenatox), naptumomab (estafenatox), narnatumab, necitumumab, nimotuzumab, nivolumab, Neuradiab® (with or without radioactive iodine), NR-LU-10, ofatumumab, olaratumab, onartuzumab, oportuzumab (monatox), oregovomab, panitumumab, patritumab, pemtumomab, pertuzumab, pritumumab, racotumomab, radretumab, ramucirumab, rilotumumab, rituximab, robatumumab, samalizumab, sibrotuzumab, siltuximab, tabalumab, tanezumab, taplitumomab (paptox), tenatumomab, teprotumumab, TGN1412, ticilimumab, trastuzumab, tremelimumab, tigatuzumab, TNX-650, tositumomab, TRBS07, tucotuzumab (celmoleukin), ublituximab, urelumab, veltuzumab, volociximab, votumumab, and zalutumumab, including antigen-binding fragments thereof.

Also provided are natural products comprising one or more compounds of the invention alone or in combination with other agents, including but not limited to therapeutic agents. In certain embodiments, the natural product is an extract or combination of extracts.

Methods and Uses

The present invention further provides methods of using one or more of the compounds of the present invention alone or in combination with other therapeutics. In particular, one or more of the compounds of the present invention alone or in combination with other therapeutics may be used in a method for treating essentially any disease or other condition in a subject which would benefit from increased surface expression of MHC-I molecules.

In some embodiments, administration of one or more compounds of the invention increases MHC-I surface expression and optionally TAP-1 expression in about or at least about 10%, 20%, 30%, 40%, 50%, 60%, 70%, 80%, 90%, or 100% of the cancer cells(s) by about or at least about 10%, 20%, 30%, 40%, 50%, 60%, 70%, 80%, 90%, or 100%, 150%, 200%, 300%, 400%, 500%, 600%, 700%, 800%, 900%, or 1000% or more relative to that of a control cell or population of control cells. In some instances, the control cell(s) are from an untreated state, for example, prior to any treatment, or from one or more earlier-treated states, for example, following a series of administrations or treatments.

In certain embodiments, the compounds of the invention alone or in combination with other therapies are used in methods of stimulating/augmenting an immune response and/or in methods of treatment of diseases associated with reduced MHC-I surface expression and/or TAP-1 expression, including but not limited to many cancers. The compounds of the invention may also be used in methods for the treatment of disorders responsive to HDAC inhibitors including psychiatric and neurological disorders such as epilepsy, depression and mood disorders. The compounds of the invention may also be used for improving general health, improving longevity and/or reducing nausea alone or in combination with other therapies. The compounds of the invention may also be used alone or in combination with other therapies in methods for treatment of infections, including but not limited to bacterial infections, including intracellular bacterial infections, viral infections such as herpes virus and parasitic diseases including protozoan and trematode infections including but not limited to schistosomiasis.

In certain embodiments, there is provided a method of augmenting an immune response involving MHC-1 CTL comprising administering one or more compounds of the invention alone or in combination with one or more other therapeutic agents.

In certain embodiments, one or more of the compounds of the invention are used alone or in combination with other therapies in a method of treating cancer. In particular, in certain embodiments, the compounds of the invention increase MHC-1 expression and optionally TAP-1 expression. Increased MHC-I surface expression and optionally increased TAP-1 expression may increase the immunogenicity of the cancer cells, and thereby increases the immune response against the cancer cells. In some instances, the immune response is a cytotoxic T lymphocyte (CTL)-mediated immune response, and can include, for example, CTL activation, clonal expansion, and increased CTL effector function. Examples of CTL effector functions include the release of release the cytotoxins perforin, granzymes, and granulysin, and increased expression of the CTL surface protein FAS ligand (FasL). In some instances, increased MHC-I surface expression and optionally increased TAP-1 expression in the cancer cell(s) increases the CTL-mediated destruction of the cancer cell(s). For solid tumors, administration of one or more curcuphenol compounds can reduce tumor expansion or reduce tumor size, for instance, by about or at least about 10%, 20%, 30%, 40%, 50%, 60%, 70%, 80%, 90%, or 100% relative to an untreated state or an earlier-treated stated.

In some embodiments, the subject has a cancer selected from one or more of breast cancer, cervical cancer, prostate cancer, gastrointestinal cancer, lung cancer, ovarian cancer, testicular cancer, head and neck cancer, bladder cancer, kidney cancer (e.g., renal cell carcinoma), soft tissue sarcoma, squamous cell carcinoma, CNS or brain cancer, melanoma, non-melanoma cancer, thyroid cancer, endometrial cancer, an epithelial tumor, bone cancer, or a hematopoietic cancer.

Examples of lung cancers include adenocarcinomas, squamous-cell lung carcinomas, small-cell lung carcinomas, and large-cell lung carcinomas.

Examples or primary bone cancers include osteosarcoma, chondrosarcoma, and the Ewing Sarcoma Family of Tumors (ESFTs).

Examples of gastrointestinal cancers include esophageal cancer, stomach (gastric) cancer, pancreatic cancer, liver cancer, gallbladder (biliary) cancer, small intestinal cancer, colorectal cancer, anal or rectal cancer, and gastrointestinal carcinoid or stromal tumors.

Examples of CNS or brain cancers include primary brain cancers and metastatic brain cancers. Particular examples of brain cancers include gliomas, meningiomas, pituitary adenomas, vestibular schwannomas, primary CNS lymphomas, neuroblastomas, and primitive neuroectodermal tumors (medulloblastomas). In some embodiments, the glioma is an astrocytoma, oligodendroglioma, ependymoma, or a choroid plexus papilloma. In some aspects, the subject has a glioblastoma multiforme. In specific aspects, the glioblastoma multiforme is a giant cell gliobastoma or a gliosarcoma. In particular embodiments, the cancer is a metastatic cancer of the CNS, for instance, a cancer that has metastasized to the brain. Examples of such cancers include, without limitation, breast cancers, lung cancers, genitourinary tract cancers, gastrointestinal tract cancers (e.g., colorectal cancers, pancreatic carcinomas), osteosarcomas, melanomas, head and neck cancers, prostate cancers (e.g., prostatic adenocarcinomas), and lymphomas.

Examples of melanomas include lentigo maligna, lentigo maligna melanoma, superficial spreading melanoma, acral lentiginous melanoma, mucosal melanoma, nodular melanoma, polypoid melanoma, desmoplastic melanoma, amelanotic melanoma, soft-tissue melanoma, and uveal melanoma.

Examples of hematopoietic cancers include lymphomas, leukemias, and multiple myelomas. In some instances, the lymphoma is a T-cell lymphoma, B-cell lymphoma, small lymphocytic lymphoma, mangle cell lymphoma, anaplastic large cell lymphoma (ALCL), follicular lymphoma, Hodgkin's lymphoma, or non-Hodgkin's lymphoma. In particular instances, the leukemia is chronic lymphocytic leukemia (CLL), hairy cell leukemia, acute lymphoblastic leukemia, myelocytic leukemia, acute myeloid or myelogenous leukemia, or chronic myelogenous leukemia.

The one or more of the compounds of the invention can be combined with other therapeutic modalities. For example, one or more compounds can be administered to a subject before, during, or after other therapeutic interventions, including symptomatic care, radiotherapy, surgery, transplantation, hormone therapy, immunotherapy, photodynamic therapy, antibiotic therapy, and administration of other therapeutic agents such as anti-cancer agents, including any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

Also provided are in vitro methods for increasing major histocompatibility complex class I (MHC-I) surface expression in a cell, comprising contacting the cell with one or more compounds of the invention or a composition that comprises the same. In some aspects, MHC-I surface expression is increased by about or at least about 10%, 20%, 30%, 40%, 50%, 60%, 70%, 80%, 90%, 100%, 150%, 200%, 300%, 400%, 500%, 600%, 700%, 800%, 900%, or 1000% or more relative to an untreated control cell.

In some embodiments, the compounds of the invention increase MHC-I surface expression by increasing the expression of Transporter associated with Antigen Processing 1 (TAP-1), a transporter protein of the MHC-I antigen presentation pathway. Hence, in certain aspects, the expression of TAP-1 is increased by about or at least about 10%, 20%, 30%, 40%, 50%, 60%, 70%, 80%, 90%, 100%, 150%, 200%, 300%, 400%, 500%, 600%, 700%, 800%, 900%, or 1000% or more relative to an untreated control cell.

In certain embodiments, the cell is a (diseased) cell characterized by reduced MHC-I surface expression (in its untreated state) relative to a non-diseased or otherwise normal or healthy cell of the same cell type. In some embodiments, reduced MHC-I surface expression in the diseased cell is associated with or caused by reduced TAP-1 expression. Hence, in some embodiments, the cell is a (diseased) cell characterized by reduced TAP-1 expression (in its untreated state) relative to a non-diseased or otherwise normal or healthy cell of the same cell type. In some embodiments, after contacting with one or more compounds of the invention, MHC-I surface expression and/or TAP-1 expression in the treated cell is increased to a level that is comparable to the MHC-I surface expression and/or TAP-1 expression of an otherwise normal or healthy cell of the same cell type. For instance, in these and related aspects, MHC-1 surface expression and/or TAP-1 expression can be increased to about or within about 50%, 40%, 30%, 20%, 10%, or 5% of the levels of MHC-1 surface expression of the otherwise normal or healthy cell of the same cell type.

In certain embodiments, the cell is a cancer cell. In specific embodiments, the cancer cell is a metastatic or invasive cancer cell. Examples of cancer cells include but are not limited to breast cancer cell, a cervical cancer cell, a prostate cancer cell, a gastrointestinal cancer cell, a lung cancer cell, an ovarian cancer cell, a testicular cancer cell, a head and neck cancer cell, a bladder cancer cell, a kidney cancer cell (e.g., renal cell carcinoma), a squamous cell carcinoma, a CNS or brain cancer cell, a melanoma cell, a non-melanoma cancer cell, a thyroid cancer cell, an endometrial cancer cell, an epithelial tumor cell, a bone cancer cell, or a hematopoietic cancer cell.

Certain embodiments employ one or more compounds of the invention or compositions comprising the same to modulate HDAC activity. Some embodiments therefore relate to method for decreasing HDAC activity in a cell, comprising contacting the cell with one or more compounds of the invention or a composition that comprises the same. In some aspects, HDAC activity is decreased by about or at least about 10%, 20%, 30%, 40%, 50%, 60%, 70%, 80%, 90%, 100%, relative to an untreated control cell. In specific embodiments, the compounds of the invention inhibit HDAC8 activity. In some embodiments, the compounds of the invention enhance HDAC activity. In specific embodiments, the compounds of the invention enhance HDAC5 and/or HDAC10 activity.

To gain a better understanding of the invention described herein, the following examples are set forth. It will be understood that these examples are intended to describe illustrative embodiments of the invention and are not intended to limit the scope of the invention in any way.

### EXAMPLES

### Example 1

The immune system is crucial in the prevention and eradication of cancer. However, cancer cells are known to mutate more frequently than normal cells and a commonly acquired phenotype is lost or reduced expression of the antigen presentation machinery (APM) that is required for immunosurveillance. This phenotype has the potential to allow cancer cells to become invisible to the immune system and metastasize with limited inhibition. This phenomenon is seen across a wide variety of cancers, discovering methods to reverse this phenotype could lead to the development of widely used anti-evasion therapeutics. A compound, curcuphenol, found in marine invertebrates as well as plants and spices, has been identified as a novel candidate for restoring expression of the APM in cancer cells. Furthermore two derivatives of curcuphenol have been synthesized which show improved outcomes, in vitro and in vivo, as anti-cancer therapeutics. Based on the structural similarity to established anti-cancer compounds, it was hypothesized that these new curcuphenol derivatives acted as histone deacetylase (HDAC) modifiers.

### MATERIALS AND METHODS

TC-1 and A9 Cell Culture

The murine lung carcinoma cell line, TC-1, was derived from primary lung epithelial cells of a C57BL/6 mouse that were immortalized using the amphotropic retrovirus vector LXSN16 carrying the Human Papillomavirus E6/E7 oncogenes and subsequently transformed with pVEJB plasmid expressing the activated human c-Has-ras oncogene. The metastatic cell line, A9, is an antecedent derivative of TC-1 that was generated in vivo after immunization of animals bearing the original TC-1 parental cells. Both cell lines were cultured in Dulbecco's modified Eagle's medium (Gibco) containing 10% fetal bovine serum (FBS, Gibco), 100 U/mL penicillin-streptomycin (Gibco) and incubated at 37° C. in a 5% CO₂ humidified atmosphere.

Western Blot

TC-1 and A9 cells were trypsinized (0.05%, Gibco) and washed with Phosphate-buffered saline pH 7.4 (PBS, Gibco). The cells were lysed in RIPA buffer (1× Tris buffered saline, Nonidet P40, 0.5% sodium deoxycholate, 0.1 sodium dodecyl sulphate (SDS), 0.004% sodium azide, Santa Cruz Biotechnologies) with HALT protease and phosphatase inhibitor cocktails (Thermo Scientific) on ice for 40 minutes with vortexing every ten minutes.

Subsequently, cells were centrifuged at 15,000×RCF for 5 minutes and supernatant was collected. Total protein was quantified using a Bradford assay and measured using the Molecular Devices Vmax kinetic micro plate reader. A total of 55 μg of protein, in 20 μL of 1× NuPAGE SDS sample buffer (Thermo Scientific) and was heated to 95° for 5 minutes, before being separated by SDS polyacrylamide electrophoresis (PAGE). Resolved samples were transferred to nitrocellulose membrane (Bio-Rad) before being blocked in 5% (w/v) skim milk with 0.2% Tween 20 (Bio-Rad). The membranes were incubated with rabbit anti-mouse TAP-1 antibody (1:1000 Jackson Immunoresearch Laboratories) and washed three times with PBS containing 2% Tween (Bio-Rad), before incubation with Alexa-Flour-680 conjugated goat anti-rabbit antibody (1:10,000, Life Technologies). Membranes were imaged on the Licor Odyssey Imaging System and quantified using Image Studio LITE (LI-COR).

Flow Cytometry

A9 and TC-1 cell lines were trypsinized (0.05%, Gibco), washed twice with PBS (Gibco), and stained with allophycocyanin (APC) conjugated anti-mouse H-2K^{b} antibody (1:200, Biolegend) suspended in 150 μL of FACS buffer (PBS+2% FBS) for 20 minutes at 4° C. Cells were washed twice with PBS and re-suspended in 200 μL FACs buffer containing 1 μL of 7-aminoactinomycin (7AAD) viability stain (Biolegend). Flow cytometry was performed on the LSRII (BDBiosciences) and analysis was done using FlowJo (Flow cytometry Analysis Software).

Immune Response of TC-1 and A9 In Vivo

To determine immune phenotype of cell lines, 5×10⁵ TC-1 or A9 cells were subcutaneously injected into the right flank of 6-8 week syngeneic female C57BL/6 (n=8), CD4^{-/-} (n=8), CD8^{-/-} (n=8) or GATA1^{-/-} (n=8) mice, giving a total number of 32 mice per cell line. Body weights were recorded three times a week following inoculation. Once tumours reached a measurable size they were calibrated three times a week and volume was calculated (V=L×W²). Mice were euthanized if they reached humane end point, based on 20% reduction in body weight, a tumour volume larger than 1 cm³ or ulceration. At the humane end point, final weights and tumour volumes were calculated before mice were euthanized and tumours were removed and weighed.

Marine Extract Library Analysis In Vitro

The marine extract library was provided by Dr. Raymond J. Andersen (UBC). The marine invertebrate specimens were collected by SCUBA diving at a 40 metre depth from regions of high marine biodiversity in Papua New Guinea, Indonesia, Thailand, Sri Lanka, Dominica, Brazil, British Columbia, South Africa, and Norway 35. Previously curcuphenol was identified as the active component in one of the marine extracts showing induction of the APM, and since then two new generations of curcuphenol analogues were synthesized in the lab of Dr. Raymond Anderson. To evaluate the ability of these compounds to induce MHC-I surface expression, A9 cells were plated in 6 well plates at 10⁵ cells/well and incubated for 24 hours at 37° C. in a 5% CO₂ humidified atmosphere. After 24 hours the medium was removed and replaced with medium containing varying concentration of synthesized compounds (6.7 μg/mL, 20 μg/mL, 60 μg/mL). One positive control TSA (100 ng/mL) and one negative control (buffer alone, 1% dimethyl sulfoxide (DMSO)), was used. Following treatment, cells were incubated for 48 hours at 37° C. with 5% CO₂ and humidified atmosphere. After incubation cells were subjected to flow cytometry.

Maximum Tolerated Dose

British Columbia Cancer Agency (BCCA) completed the maximum tolerated dose study for compound P02-113, whereas P03-97-1 was assessed in-house following the same protocol. A total of nine C57B/6 female mice between the ages of 6-8 weeks were used for each compound. The compounds were injected intraperitoneally (i.p.) at concentrations of 1.0 mg/kg (n=3), 3.5 mg/kg (n=3), or 5.2 mg/kg (n=3). These doses were based on the maximum solubility of the compounds that was determined using the known solubility of curcuphenol. Mice were assessed for clinical signs of toxicity for 14 days following injection. After 14 days the mice were euthanized and examined by necropsy, P02-113 was performed at BCCA, and P03-97-1 was performed by Animal Care Services located at the Center for Comparative Medicine on UBC Point Grey campus, Vancouver BC.

Pharmacokinetics

To assess the pharmacokinetics (PK) of the curcuphenol analogues, a mass spectrometry assay was developed to measure the compounds in plasma. This assay was created by The Metabolomics Innovation Center (TMIC) at UVic-Genome BC Proteomics Centre located in Victoria, British Columbia. Eight samples were sent to TMIC for PK design for identification of P02-113 and P03-97-1 in mouse plasma. To collect plasma, mice were anesthetized using isoflurane and blood was collected by cardiac puncture. Plasma was isolated from blood in a potassium-EDTA coated Tube with K2E (BD Microtainer) and centrifugation at 10,000×g for 1 minute. Plasma was transferred to a cryovial and stored at −20° C. before being shipped on dry ice. TMIC used a chemical derivatization—UPLC-MMR/MS method to create a quantitative analysis tool for the compounds using dansyl chloride (DAN.CI) as the derivatizing reagent. 13C-labeled DAN.CI was used to produce stable isotope-labeled internal standards (ISs). All tests were performed using an UPLC-4000 QTRAP system with ESI and (+) ion detection using C18 column and acetonitrile-water-formic acid as the mobile phase.

For the PK analysis of P02-113 and P03-97-1, mice were injected i.p. and anesthetized before blood was collected by cardiac puncture at five time points (5 min, 10 min, 30 min, 1 hour and 6 hour). Time points were chosen based on the published data for TSA (0.5 mg/kg), a drug of similar chemical structure and size to the compounds. Three female C57BL/6 mice, between the ages 6-8 weeks, were used for each compound for each time point, giving a total 12 mice per compound. All mice were injected at the highest maximum tolerated dose (5.2 mg/kg) and plasma was prepared and stored as previously described above.

In Vivo Tumour Trial

The metastatic cell line, A9, was grown in the DMEM, as previously described without, the addition of antibiotics (penicillin and streptomycin, P/S). Once cells reached 75-80% confluence, they were trypsinized (0.05%, Gibco) and washed with HBSS (Hanks balanced salt solution). The cells were counted using the Bio-RAD TC20 automated cell counter and suspended to a concentration of 10⁷ cells/mL in HBSS. Thirty-two synergistic female C57BL/6 mice, between the ages 6-8 weeks, were subcutaneously injected in the right flank with 50 μL containing 5×10⁴ A9 metastatic tumour cells. Seven days following tumour inoculation i.p. treatment began daily for 12 days. Four treatment groups were studied, with eight animals per group. The vehicle was used for a negative control (1% DMSO in PBS), TSA (0.5 mg/kg) a drug known to reduce A9 tumour burden in vivo (11), P02-113 (5.2 mg/kg) and P03-97-1 (5.2 mg/kg), were evaluated. Body weights were measured three times a week and once tumours developed they were measured with calipers and tumour volume was calculated. Twelve days after starting treatment, mice were euthanized and tumours were collected and weighed. The tumours were then processed for flow cytometry analysis. Tumours were cut into small pieces and incubated RPMI (Gibco; with P/S 0.5%, Sodium pyruvate 1%, and L-glutamine 1%) and 3 mg/mL collagenase A (Roche) for one hour at 37° C. with shaking. Dissociated tumour cells were passed through a 100 μm filter and spun down at 15,000×RCF for 3 minutes. The pellet was washed once in FACs buffer (2% FBS in PBS) and spun down. The pellet was next suspended in red blood cell (RBC) lysis buffer and kept at room temperature for 5 minutes before being neutralized by the addition of 5 ml of PBS and spun down. If pellets were still found to contain RBCs this step was repeated. Once all RBCs were removed cells were suspended in FACs buffer to a concentration of 10⁷ cells/mL. A total volume of 200 ul of cells from each tumour were added to a 96 well plate (Falcon) and incubated with Fc Blocker (Biolegend, 1:400) for twenty minutes at 4° C. The 96 well plate was spun down at 1,200 rmp for three minutes and supernatant was removed. The cells were then suspended in 150 ul of FACs buffer containing anti-CD8a (PE-Cy7, 1:200, eBioscience) and CD4 (APC, 1:200, Biolegend) antibodies and incubated at 4° Celsius for 20 minutes. The cells were washed twice and spun down using FACs buffer before being to flow cytometry tubes in a final volume of 200 ul of FACs buffer containing 7AAD (Biolegend 1:200). Flow cytometry was performed on LSRII (BDBiosciences) and analysis was done using FlowJo (Flow cytometry Analysis Software).

HDAC Assays

Compounds P02-113 and P03-97-1 were analyzed for their effect on histone deacetylase activity in the A9 cell line, using the HDAC-Glo™ I/II Assay and Screening System (Promega). The linear range was established for the A9 cells in a black-walled, clear-bottomed 96 well plate (PerkinElmer). Cells were diluted to 10⁵ cells/mL and serial diluted by two fold, to a final concentration of 98 cells/ml. All dilutions were plated in triplicates in a volume of 100 ul per well. Cells were left overnight at 37° C. for 24 hours before addition of HDAC class I/II reagent. Luminescence was read after 30 minute incubation with HDAC class I/II reagent. After determination of an optimal cell density of 30,000 cells/well, cells were plated in 96 well plate and left for 24 hours at 37° C. Media was used as a blank control, as well a positive control was included consisting of HeLa cells provided in the HDAC assay kit. The next day, media was removed from the wells and new media containing vehicle (negative control), TSA (positive control), or a range of dilutions of P02-113 or P03-97-1 (5 to 0.02 μM) was added in triplicates and incubated for 30 minutes. HDAC class I/II reagent was then added and incubated for 30 minutes before luminescence was measured using the Infinite M200 (Tecan) and i-control software (Tecan).

Individualized HDAC Assays

The activity of the curcuphenol analogues was assessed with purified HDAC enzymes from all classes I, II, and IV, as well as a select member of HDAC class III (SIRT1). HDACs 1-9 and SIRT1 were evaluated using HDAC Fluorogenic Assay Kits (BPS Biosciences). All assays were completed in black-sided clear-bottom 96 well plates (PerkinElmer), and all treatments were plated in triplicates. Treatment started at 5 μM and was two-fold diluted to a concentration 0.02 μM. The assays were measured using the Synergy HI hybrid reader (BioTek) and Gen5 software (BioTek), excitation was set to 360 nm and detection was measured at 450 nm with a gain of 100. Alternatively HDAC 10 and 11 (BPS Biosciences) were optimized for HDAC concentration using the HDAC-Glo™ I/II Assay and Screening System (Promega). Following optimization each HDAC was run following the Promega protocol in black-sided clear-bottomed 96 well plates in triplicates with same treatments listed above (PerkinElmer). Luminescence was read 30 minutes after HDAC-Glo™I/II reagent was added using the Synergy HI hybrid reader (BioTek) and Gen5 software (Bio-Tek). For all assays, vehicle (1% DMSO) was used as a negative control and TSA (25 nM) was used as a positive control, excluding SIRT1 where nicotinamide (5 mM) was used as positive control, and all assays contained multiple blank controls. To calculate percent activity, the average of blank wells was subtracted from all treatment groups. The relative mean of activity of the HDAC being measured was determined and all wells that received treatment were divided this average, to give a percentage of activity.

Plasma Samples Sent for Development of Pharmacokinetic Assay.

| | CONCEN- |
|---|---|
| | TRATION |
| SAMPLE | (mg/mL) |
| Plasma from untreated mouse | 0 |
| Plasma from untreated mouse with P02-113 added | 10 |
| Plasma from untreated mouse with P03-97-1 added | 10 |
| Plasma from mouse injected with 100 μL of P02-113 | 10 |
| Plasma from mouse injected with 100 μL of P03-97-1 | 10 |
| 100 μL of P02-113 in 100% DMSO | 13 |
| 100 μL of P03-97-1 in 100% DMSO | 13 |

Results

Characterization of the TC-1 and A9 Cell Lines

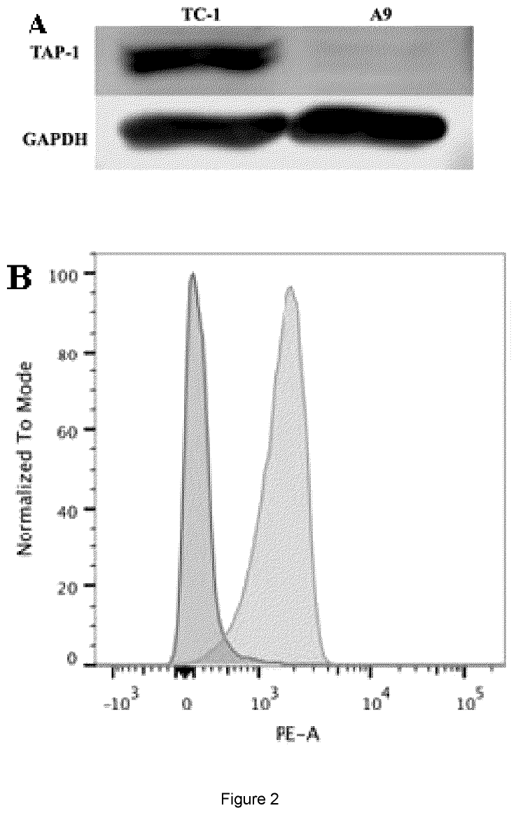

The murine metastatic lung carcinoma cell line, A9, was chosen for the analysis of small molecules to recover an immunological phenotype because it is known to have reduced expression of the APM (FIG. 2(A) (9-11). The metastatic A9 cell line was derived from a murine primary lung carcinoma, TC-1 that retains expression of the APM, by passaging in vivo.

Immune Response In Vivo

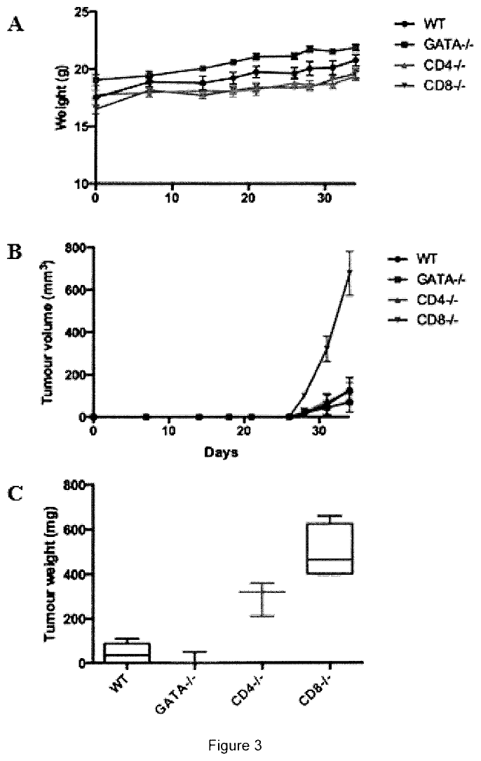

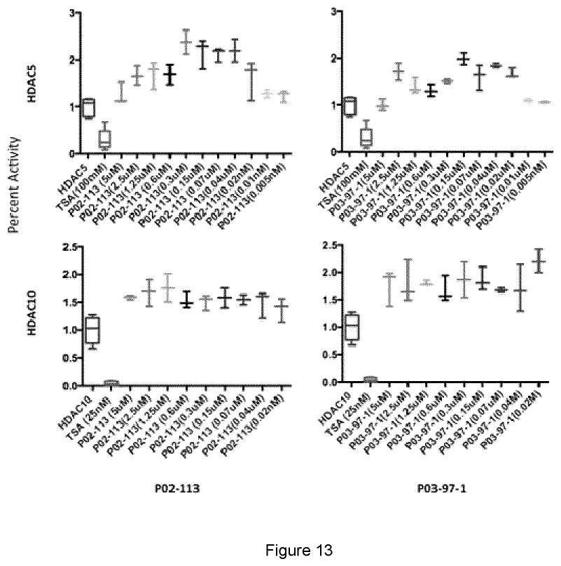

To determine if there was a difference in immune response between primary and metastatic cell lines in vivo, 5×10⁵ TC-1 cells were subcutaneously injected into the right flank of a variety of 6-8 week old, synergistic mouse models. To assess the induction of both the endogenous and exogenous pathways of the APM, mice lacking either CTLs (CD8^{-/-}, n=8) or T helper cells (CD4^{-/-}, n=8) were inoculated. A control mouse, with a fully capable immune system (wild type C57BL/6 mice, n=8), was also included as well mice lacking eosinophils (GATA1^{-/-}, n=8) that are a class of immune cells known to play a role in the tumour response. All mice inoculated with the TC-1 cell line were weighed three times a week throughout the study, and it was found that all mice gained weight at a healthy rate with no significance difference between any of the four groups (FIG. 13.2A). Of the four-mouse strains mice lacking CTLs developed the largest tumours in comparison to the wild type controls (FIGS. 3B&C), demonstrating that the CTLs play a crucial role in recognizing the TC-1 cells and reducing overall tumour burden. This was as hypothesized as CTLs cells interact with cancer cells via the MHC-I molecules, validating the important role of the endogenous antigen pathway in adaptive immune systems' identification and elimination of cancer cells. The mouse model lacking T helper cells, representing the exogenous APM that acts through MHC-II molecules, also showed a more significant tumour volume than the wild type controls (FIGS. 3B&C). A possible explanation for this difference is that the T helper cells are known to help maintain CTL activity after initial activation, and upon removal of the T helper cells the CTLs may have lost a significant amount of activity. The final mouse model examined, lacking eosinophils, was found to have a reduced tumour burden in comparison to the wild type mice, however this difference was not found to be statistically significant. However, the role of eosinophil's has been largely controversial in regard to tumours which largely depends of the tumour type.

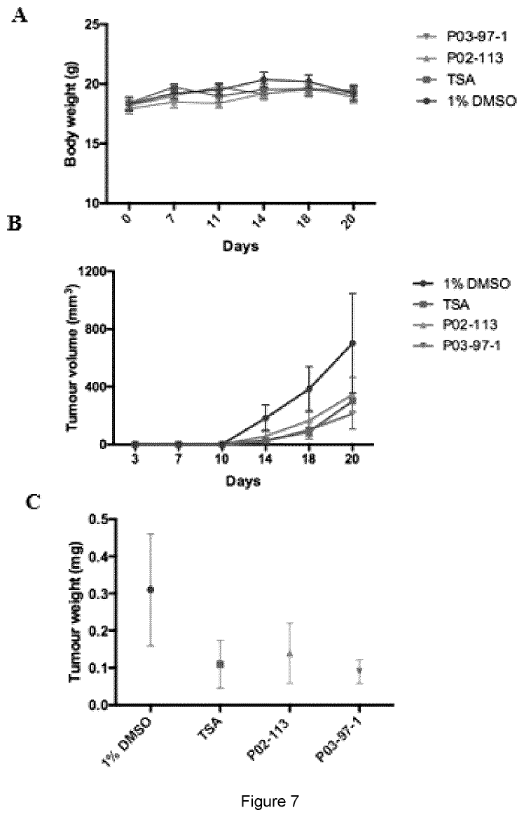

The same mouse experiment was performed using the A9 cell line. Mice of all four genotypes developed tumours at a similar rate when inoculated with A9. However, due to the aggressive nature of the A9 cell line, several mice developed ulcerations and had to be euthanized and to keep the time of tumour growth consistent, all mice were sacrificed on day 14. Of the mouse models examined, only mice lacking T helper cells showed a difference in tumour burden (FIG. 7). A possible explanation for this result is the response of T helper cells to professional antigen presenting cells located in the tumour microenvironment. Therefore, without T helper cells present, they cannot stimulate an immune response. In regard to the TC-1 experiment, it validates that exogenous APP which utilizes MHC-II and T helper cells may also be crucial for an immune response in these cell lines. As for the other knockout models examined, there was no significant difference in tumour burden in comparison to the wild type mice (FIG. 7) demonstrating that eosinophil's are not involved in response to these cells lines and that MHC-1 and TAP-1 expression is required for a CTL response in vivo.

Screening Small Molecules for Induction of MHCI

Two generations of curcuphenol analogues were evaluated for their ability to induce MHC-I surface expression in vitro. Analogues showing the greatest induction of MHC-I and lowest cytotoxicity were further examined for effects on tumour growth in vivo.

Identifying Analogues that Induce the Antigen Presentation In Vitro

Previously, marine invertebrate extracts collected from oceans around the world were screened for the ability to induce TAP-1 and MHC-I expression in the A9 metastatic cell line, using cellomics and flow cytometry (72). After identification of extracts with substantial stimulation of these APM components, selected extracts were fractionated by separation chromatography and HPLC into aqueous and ethanol fractions, in the lab of Dr. Raymond Anderson (Department of Chemistry, UBC). After fractionation, extracts were again screened for the ability to induce MHC-I expression in A9 cells. From these screens one fraction showed a significantly stronger induction of the APM components compared to all other tested fractions. The active component was identified as S-(+)-curcuphenol by NMR in the lab of Dr. Raymond Anderson. While curcuphenol was isolated from a marine invertebrate, it is also found in plants and spices, and its enantiomer, R-(−)-curcuphenol, is also found in several marine invertebrates.

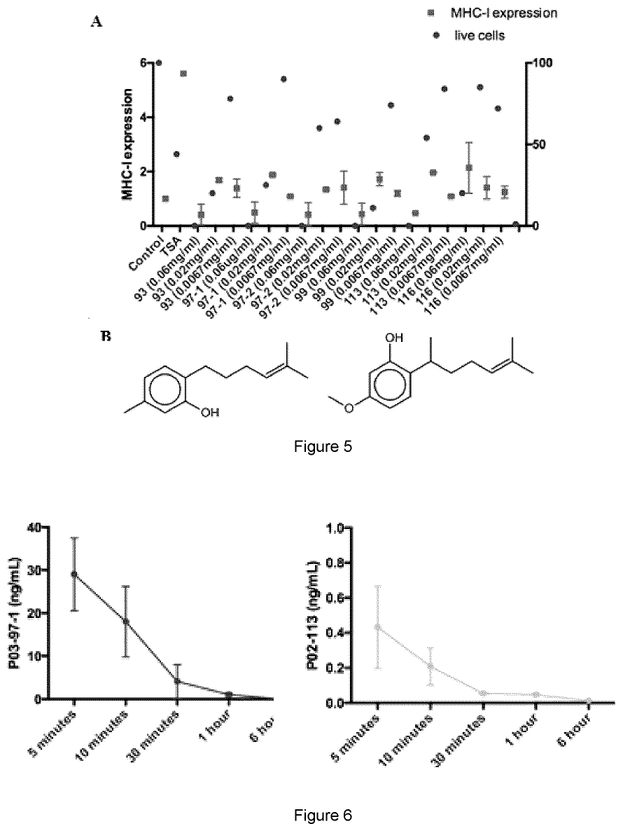

While curcuphenol was isolated from a sea sponge extract in the pure S form laboratory synthesis of curcuphenol results in a racemic mixture, necessitating cumbersome separation methods. Instead, we opted for the synthesis of analogues lacking the chiral center and two generations of curcuphenol analogues were synthesized in the lab of Dr. Raymond Anderson. The first generation was modified by structural changes to the carbon tail, P02-113 and P02-116, whereas the second generation contained modifications on both the carbon tail as well as the carbon ring, P03-93, P03-97-1, P03-97-2 and P03-99. I screened these compounds by flow cytometry for the ability to induce MHC-I expression at the cell surface of A9 cells while maintaining a low level of cytotoxicity (FIG. 5A). Two analogues, P02-113 and P03-97-1, were particularly interesting due to their reproducibility for strong induction of MHC-I while maintaining low cytotoxicity (FIG. 5B).

Maximum Tolerated Dose

To determine the maximum tolerated dose of the curcuphenol analogues, P02-113 and P03-97-1, they were evaluated for toxicity at multiple concentrations. Concentrations started at 1.0 mg/kg for both compounds, followed by 3.5 mg/kg and a final concentration of 5.2 mg/kg. Solubility of the compounds was the limiting factor in this trial as 1% DMSO is the highest concentration approved when using i.p. injection. Based on these restrictions, 5.2 mg/kg was the highest dose we could inject by i.p. Three mice were evaluated at each concentration for both compounds, giving nine mice per compound. Mice were monitored for 14 days and no clinical signs of cytotoxicity were seen. After 14 days, mice were subjected to necropsy. At all concentrations both compounds showed no signs of toxicity or abnormalities to be reported. Therefore 5.2 mg/kg was chosen for dosing in future experiments.

Pharmacokinetic of P02-113 and P03-97-1

To determine the dosage regiment for treatment of mice the pharmacokinetics of P02-113 and P03-97-1 were monitored after i.p injection at varying time points. Time points were chosen based on literature from a structurally similarity compound, TSA, which becomes metabolized between 5 and 60 minutes with a half-life just under ten minutes and no detection after 24 hours (73). While the analogues are similar in structure to each other, they were significantly different in their metabolism. P03-97-1 was found at a concentration 30 ng/mL in mouse plasma after 5 minutes and was approximated to be at half this concentration around 20 minutes based of the 10 and 30 minutes time points. Alternatively, P02-113 was found at a concentration of 0.4 ng/mL after 5 minutes and was reduced to half of this concentration after 10 minutes. Therefore it was calculated that P03-97-1 has a half-life of 15 minutes while P02-113 has a half-life of less than 5 minutes. Due to time limitations in the ability to inject mice and collect blood no time points earlier then 5 minutes were possible. Another limitation was that each time point required one mouse to get sufficient plasma for PK sampling, therefore one mouse could not be used for multiple time points. Both compounds were consistent in that they reached undetectable levels in mouse plasma at the 6 hour time point. Due to the high eliminations in the mouse plasma similar to TSA, which is effective upon daily dosing, as well in limitation dosing regimes mice were chosen to be treated daily.

Evaluation of Small Molecules, P02-113 and P03-97-1, In Vivo

To evaluate the ability of the small molecules to stimulate the immune system in vivo, A9 cells were subcutaneously injected into the right flank of 32 6-8 week year old C57BL/6 mice at a concentration of 4×10⁵ cells/mouse. Seven days after inoculation mice were randomized into one of four treatment groups (n=8): vehicle (1% DMSO), TSA (0.5 mg/kg), P02-113 (5.2.mg/kg), or P03-97-1 (5.2 mg/kg), and treated daily by i.p. for 12 days. The body weights and tumour volumes were measured three times a week throughout the entire study. In all four treatment groups, body weights remained stable throughout the study (FIG. 7). The tumour volumes (FIG. 7B) were reduced in all treatment groups, TSA, P02-113 and P03-97-1, compared to the vehicle control. Tumour weights (FIG. 7C) were measured at the end point and found to agree with final tumour volume data collected at the end of the study. Of the three treatments, P03-97-1 had a significant anti-tumour effect with a p-value of 0.0001, that was more significant then the positive control TSA with a p-value of 0.0012, calculated using a paired one-tailed t-test. P02-113 also showed an inhibition on tumour growth but was not found to be as significant as P03-97-1 or TSA.

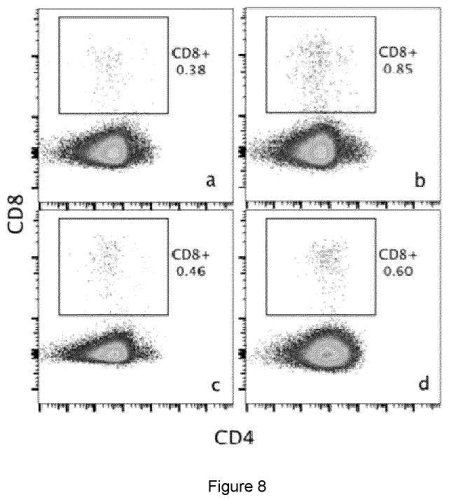

Tumours were also subject to analysis for T cell infiltration at the study end point. Tumours were analyzed by flow cytometry for CD4+ (APC) and CD8+ (PE-Cy7) T cells (FIG. 8). Interesting, the infiltration of CD8+ T cells followed a similar pattern to what was seen in tumour burden. TSA and P03-97-1 had the greatest CD8+ infiltration followed by P02-113 and vehicle alone. As for the CD4+ there was no significant infiltration or difference in any of the groups. These results suggest that P03-97-1 a stronger immunological stimulator in vivo and also exhibited the greater reduction in tumour burden, suggesting that future studies should focus on optimizing the structure of P03-97-1.

Class I/II Histone Deacetylase Activity

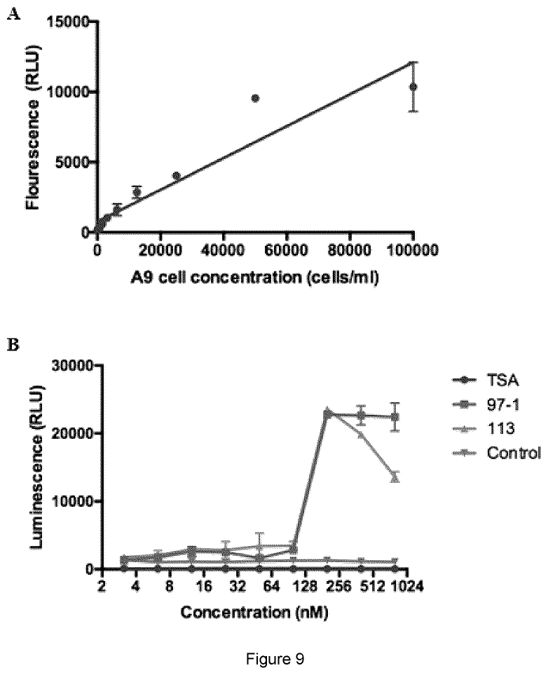

Due to similarity of the curcuphenol analogues, P02-113 and P03-97-1, to a previously described HDACi, TSA, it was hypothesized that these molecules could be acting through a similar mechanism. To test this theory, P02-113 and P03-97-1 were analyzed in the A9 cell line using a general HDAC-Glo™ I/II Assay and Screening System (Promega). First, the linear range of HDAC enzyme activities in the A9 cell line was determined for optimal fluorescence reading in the assay, and a density of 30,000 cells/mL was selected (FIG. 9A). Following optimization, the small molecules were tested in a range of concentrations (1 nM-1 μM) on the A9 cell following the assay protocol and luminescence was determined. Interestingly the compounds, P02-113 and P03-97-1 exhibited the opposite effect to what was hypothesized and showed an increase in class I/II HDAC activity (FIG. 9B). Even at the lowest concentrations, 1 nM-100 nM, there was an induction of HDAC activity. Both compounds showed a peak in HDAC activity around 180 nM, while P02-113 started to reduce it effect at higher concentrations. P03-97-1 maintained peak levels of HDAC activity until the highest concentration of 1 uM suggesting a stronger effect. The stronger effect exhibited by P03-97-1 could be due to several factors including stronger binding affinities to HDAC enzymes, or better ability to enter A9 cells, however the exact reason remains to be determined.

Class I Histone Deacetylase Activity

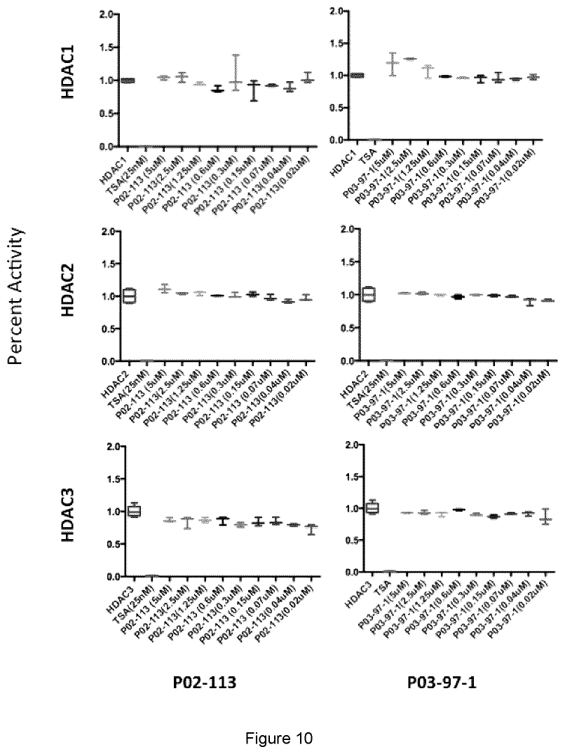

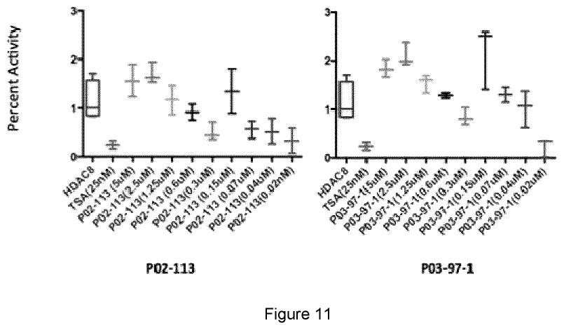

In the class I HDAC family there are four HDACs, 1,2,3 and 8. Of the class I tested HDACs, 1-3 showed no significant change in HDAC activity at the concentrations tested for both P02-113 and P03-97-1 (FIG. 10). For compound P02-113, HDAC8 showed more variable results with no change in HDAC8 activity at higher concentrations but at concentrations of 0.3 uM and below inhibition was seen, that was similar to the HDACi exhibited by TSA. P03-97-1 also followed a similar pattern with no change in activity at higher concentrations but at the lowest concentration 0.02 uM an inhibitory phenotype was seen. This indicates that the analogues P02-113 and P03-97-1 act as inhibitors to HDAC8 but not for other class I enzymes. Another interesting factor that correlates with the inhibitory effects of P02-113 and PO-3-97-1, is that HDACs 1-3 are limited to the nucleus whereas HDAC8 is the only class I also found in the cytosol.

Class II Histone Deacetylase Activity

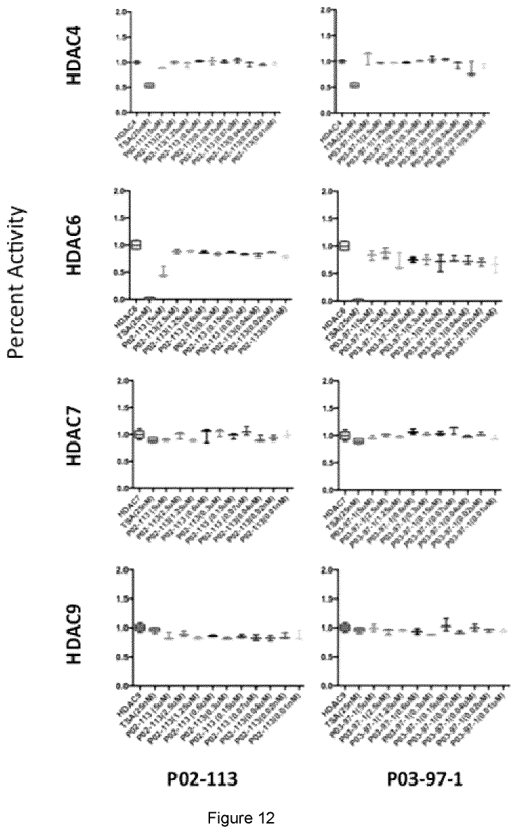

The class II HDAC family encompasses HDACs 4 through 10 excluding HDAC8. All class II HDACs were evaluated with compounds P02-113 and P03-97-1 at concentrations ranging from 0.02 to 5 μM. Of the class II HDACs those that showed no change in activity upon treatment were HDACs 4, 6, 7 and 9 (FIG. 12). Of these HDACs, it is also noteworthy that although TSA was used as a positive control it is known, that TSA has a limited effect on HDACs 6, 7 and 9, indicating these HDACs may have more unique structures making them a harder target when looking for compounds that alter HDAC activity. Alternatively both HDAC 5 and 10 were enhanced upon treatment with either of the curcuphenol analogues (FIG. 13). For HDAC5, it was seen that enhancement was limited to concentrations between the range 2.5-0.01 μM, for both analogues. HDAC10 however was seen to be enhanced at all concentration between 5-0.02 μM, indicating that a wider concentration range is needed to determine the limits of dosage on HDAC10 activity.

Class III Histone Deacetylase Activity

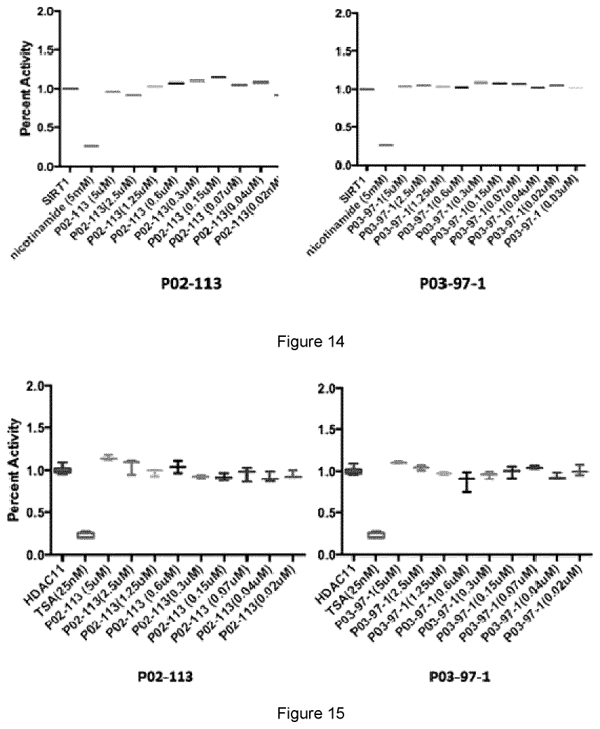

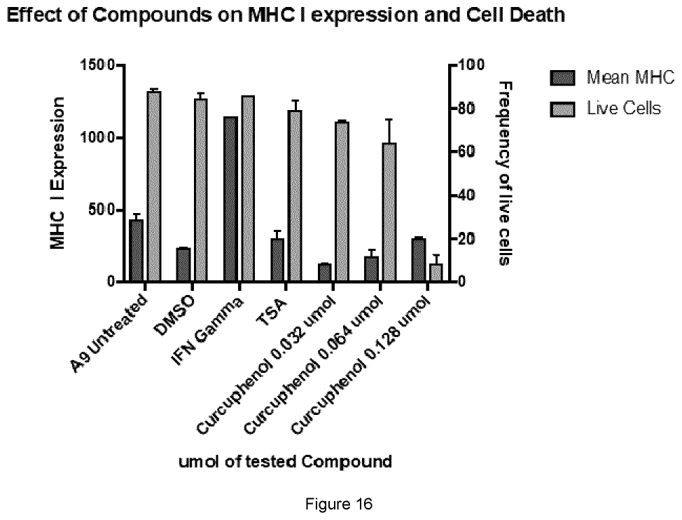

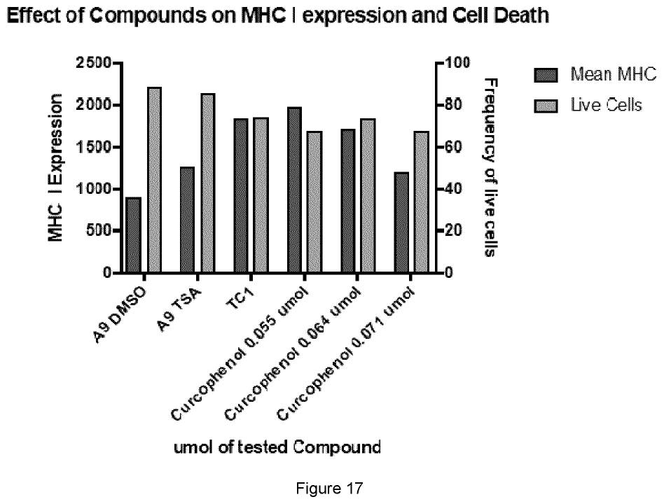

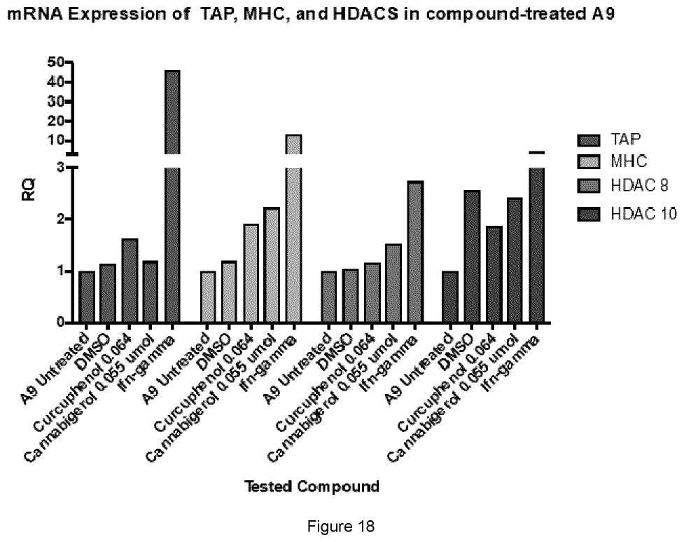

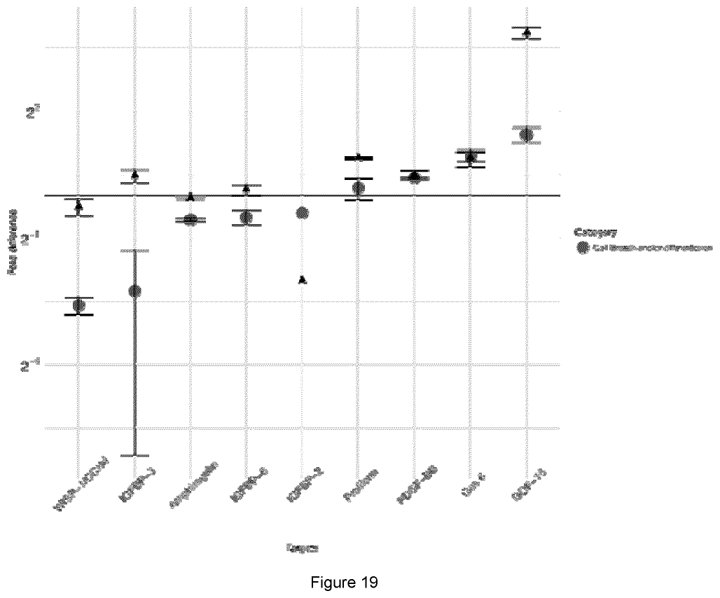

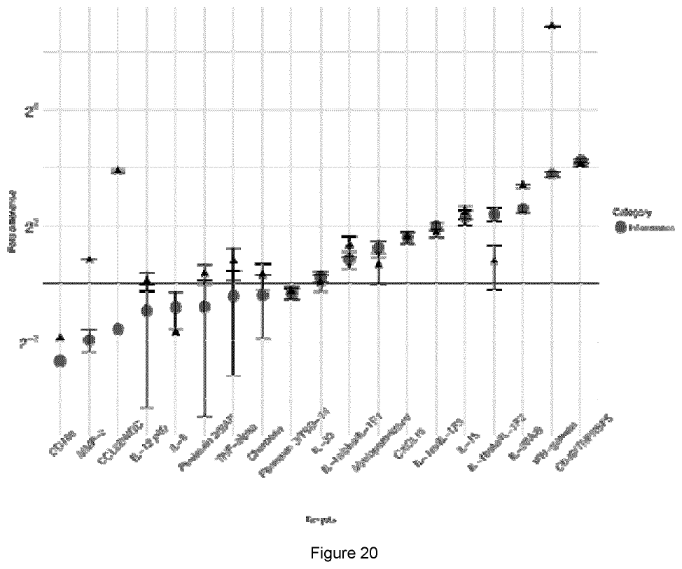

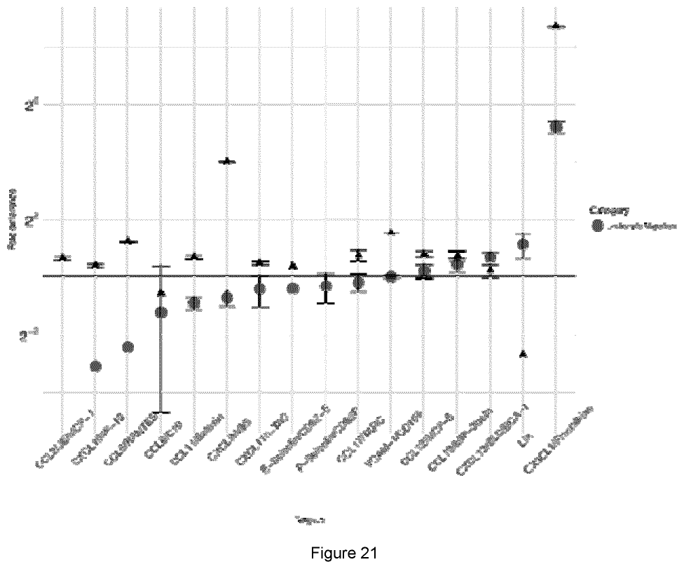

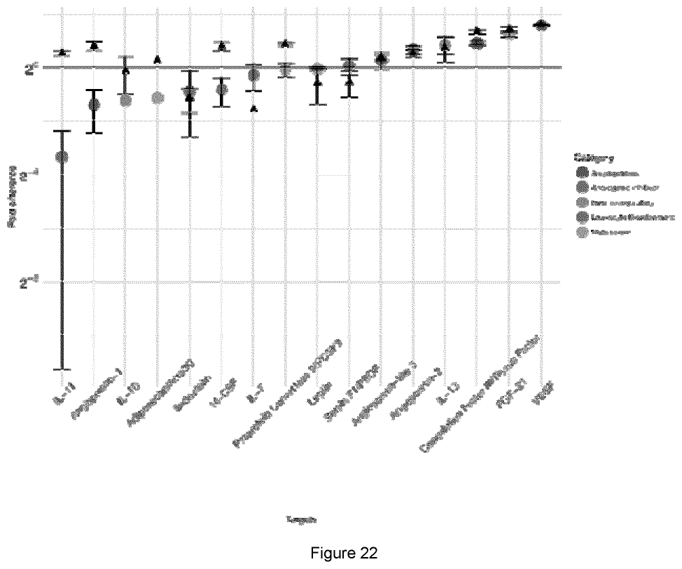

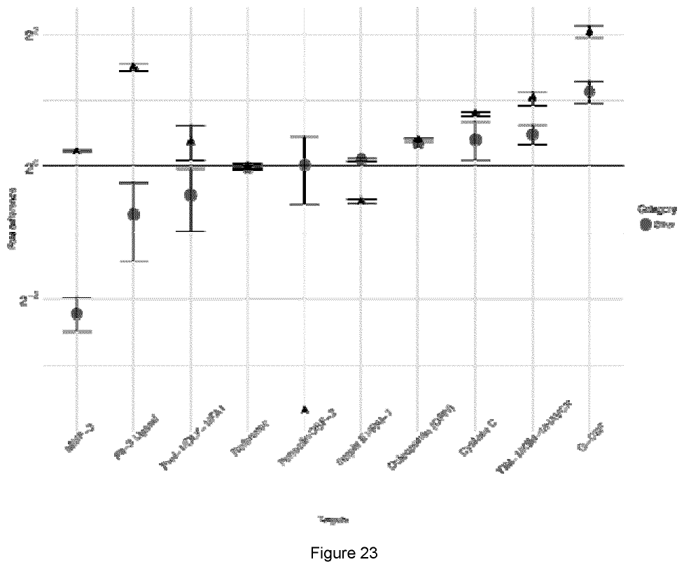

There has yet to be an HDACi that has an effect on the class III enzymes, therefore only one enzyme was selected for analysis of activity upon treatment with the two analogues. SIRT1 was chosen because is the only class III that is known to play a role in carcinogenesis. SIRT1 was treated with compounds P02-113 and P03-97-1 at the same concentration range stated in previous assays. SIRT1 did not demonstrate a change in activity upon treatment (FIG. 14). Due to this result and strong similarity in structure to other class III enzymes further class III enzymes were not tested.

Class IV Histone Deacetylase Activity

The activity of HDAC11, the only class IV enzyme, was unaffected upon treatment with either analogues, P02-113 or P03-97-1 between the range of 5 μM and 0.02 μM (FIG. 15). Indicating that the compounds neither enhance nor reduce the activity of HDAC11 at the examined concentrations.

### DISCUSSION

Immune Response to TC-1 and A9 Cell Lines In Vivo

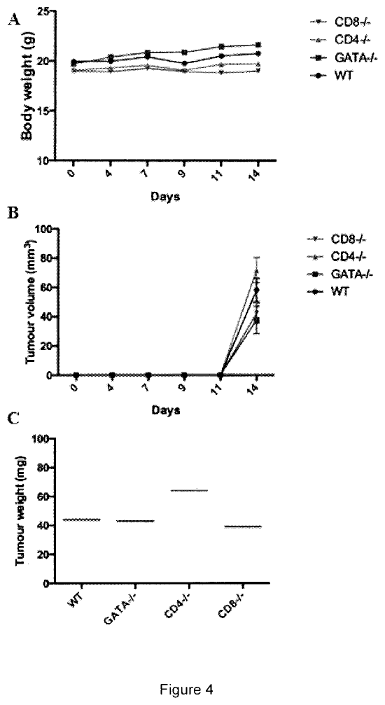

The immune system is responsible for the recognition and elimination of cancerous cells. While both arms of the immune system, innate and adaptive, participate in this process, the endogenous APM of the adaptive immune is of particular importance. The endogenous APM allows the TCR present on the surface of CTLs to recognize MHC-I molecules present on the surface of all nucleated cells and determine if an adaptive immune response should be initiated. Due to the importance of this pathway in adaptive immune surveillance many cancers down-regulate components involved in the endogenous APP. Of the different proteins involved in the APP TAP-1 and MHC-I are the most frequently down-regulated and approach 100% reduction of expression in some carcinomas (9-11, 23, 24). Since the A9 metastatic cell line has reduced expression of both TAP-1 and MHC-I, in comparison to its primary counterpart TC-1, the immune response between the two cell lines was hypothesized to be significantly different in vivo. Due to the lack of expression of TAP-1 and MHC-I in the A9 cell line it was evident that these cells had a clear growth advantage in wild type mice in contrast to the TC-1 cell line. The A9 tumours became measurable 14 days after inoculation (FIG. 4), almost twice as fast as the TC-1 tumours that were measurable after day 25 (FIG. 3). The A9 cells were also found to be significantly more aggressive as the mice had to be taken down at a much earlier time point due to ulceration.

To further specify if a difference in tumour growth is attributed to the APM, specifically recognition of tumour cells by CTLs, both cell lines were evaluated for tumour growth in different mouse models lacking varying components of the immune system alongside wild type mice. The mouse models chosen were mice without CTLs (CD8^{-/-}) representing the endogenous APP, mice without T helper cells (CD4^{-/-}) representing the exogenous APP, and mice without eosinophil's (GATA1^{-/-}) which are known to play a role in cancer elimination. As predicted, the TC-1 cell line showed a significantly faster tumour growth rate in mice lacking CTLs as compared to the C57BL/6 wild type control (FIG. 6). While the TC-1 cells retain the expression of TAP-1 and MHC-I the mice lacking CTLs, are unable to recognize the MHC-I molecules and therefore cannot initiate an appropriate immune response. Interestingly the mice without T helper cells, representing the exogenous APP, also showed a difference in tumour weight compared to wild type mice, indicating that they are contributing the reduction of TC-1 tumour burden. A possible explanation for these results is that T helper cells are known to play a role in maintaining CTL activity after initial activation by cancer cells, therefore with no T helpers cells present, CTL activity may be greatly reduced resulting in faster tumour growth. As for the mice lacking eosinophil's, there was no change in growth compared to wild type mice indicating these cells do no play a role in recognition of the TC-1 cell line. This disagrees with the current notion that eosinophil's play a role in the reduction of tumour burden, however new research is frequently starting to show that eosinophil's role in cancer is largely dependent on the cancer type (79). While the mechanisms by which eosinophils promote cancer have yet to be explained, in multiple human studies hyper eosinophilia has been associated with poor prognosis (80,81). Overall from this experiment, it is clear that the immune is utilizing CTLs as a primary defense to detect and eliminate TC-1 cancer cells and that T helper cells may also be crucial in maintaining such defense.

The same in vivo experiment was performed using the A9 cell line with the hypothesis that there would be no significant difference in tumour growth between wild type and any of the three knockout mouse lines. As for the mouse models examined there was no significant difference seen except in the mice lacking T helper cells, which had more aggressive tumours (FIG. 7). This may be attributed to their role in responding to professional antigen presenting cells that present exogenous peptides through MHC-II in the tumour environment to the T helper cells. To confirm the role of T helper cells as well as the lack of role of other immune cells evaluated, a longer study longer than 20 days will be needed to confirm is this difference is consistent over the long term by using fewer A9 cells in vivo. As well future studies should also include other immune mouse knockout models, such as natural killer cells and macrophages, to rule out any other immune cell types that may be involved in recognition of the either the TC-1 or A9 cell line.

Therapeutic Potential

Since it was discovered that the immune system plays an essential role in reducing the occurrence and severity of cancers, the field of cancer immunotherapy has significantly grown in the last decade (82, 83). Cancer immunotherapy works by initiating an immune response against the invading cancer cells. Currently there are several cancer immunotherapeutic agents in development, including small molecules such as monoclonal antibodies (mAbs), vaccines and cytokines as well as cellular therapies such as adoptive cellular therapy (ACT) (82, 83). Of the small molecules mAbs have shown the greatest potential and are often targeted against immune cells opposed to cancer cells allowing them to treat a range of cancer types (82). Antibodies are often used to target programmed cell-death protein 1 (PD-1) or cytotoxic T-lymphocyte protein 4 (CTLA-4) both located on surface of T lymphocytes and function as inhibitory receptors involved in immune checkpoint signaling (82). By blocking either of these receptors with antibodies cancer cells are no longer able to inhibit T lymphocyte activation via their corresponding receptors. Alternatively to small molecules, ACT works by ex vivo manipulation and expansion of T-lymphocytes to target the cancer cells (82). There are currently several techniques under-development including the selection and expansion of tumour infiltrating lymphocytes (TILs), gene transfer of a synthetic TCR (sTCR) or a chimeric antigen receptor (CAR) into T cells (82). Interestingly many of these therapies have shown great potential but in several cases there are still a significant amount of patients that show no response (82,83). Of the patients experiencing no benefit from such therapies it has been predicted that a percentage of the patient's cancers remain unaffected due to deficiencies in the APM (82). Therefore combination therapies may be key in the future, where the addition of drugs targeting the up regulation of the APM will be utilized (83). The curcuphenol analogues, P02-113 and P03-97-1, have demonstrated optimal effects on tumour burden in vivo and may be optimal choices for combination therapies as they induce MHC-I expression and in combination with other therapies could greatly increase the outcome for patients whose cancers show a immune evasive phenotype due to reduced levels of the APM. However optimization of the dosing of P02-113 and P03-97-1 will be required as it was seen they have a high rate of elimination from the blood stream as they are both undetectable after 6 hours. To increase therapeutic potential an increased dosing regimen may be needed. Alternatively, a different route of administration may overcome the solubility limitation encountered when using i.p. for treatment. Furthermore experimenting with the chemical structures of the compounds may lead to more potent or soluble compounds that could also lead to increased potential for therapeutics.

Histone Deacetylase Activity of P02-113 or P03-97-1

Due to the very similar structure of the curcuphenol analogues to a known HDACi, TSA, which promotes the expression of MHC-I in the A9 cell line (9-11) it was predicted that the analogues were acting through a similar mechanism. However, upon a generalized class I/II HDAC luminescence assay to measure HDAC activity, using A9 cells, the opposite effect was discovered and HDAC activity was enhanced. This HDAC enhancement (HDACe) is a novel trait that has never been seen in the literature for class I/II HDACs, however, there is one known HDAC activator for the class III HDACs, reversitol, which indirectly acts upon SIRT1 (84). To determine if P02-113 and P03-97-1 were in fact directly interacting with HDAC enzymes to promote activity, individual purified recombinant HDACs were assessed following treatment with the analogues. While the majority of HDAC enzymes did not show a change in activity, one enzyme, HDAC8, showed inhibition. This is interesting as this if the only class I HDAC that is known to exist in both the nucleus and cytoplasm and diverged early in evolution from the other class I enzymes (85). This very specific targeted inhibition of HDAC8 is a unique feature of the compounds as the majority of HDACi being developed show pan HDACi. However these analogues present a more targeted and optimal affinity then has been seen before. Luckily it is known that increased HDAC8 activity is associated with cancer as well as in other diseases including neurodegenerative disorders, metabolic deregulation, autoimmune and inflammatory diseases (85). Therefore these compounds hold potential as specific HDAC8 inhibitors. In regard to the APM it has been demonstrated that HDAC8 acts as a scaffold for cAMP responsive element binding protein (CREB), a known transcriptional up-regulator of TAP-1 and MHC-1 (82). One study showed upon over-expression of HDAC8, CREB phosphorylation became decreased along with its transcriptional activity (86). To determine if the increased expression of the APM is directly correlated with the inhibitory activity of P02-113 and P03-97-1 on HDAC8 further experiments in which HDAC8 is knocked down in the TC-1 cell line and APM expression is measured will be required. HDAC8 has previously been knocked-down using RNA interference in lung, colon and cervical cancer cell lines resulting in reduced proliferation while its over-expression promotes proliferation and inhibits apoptosis in hepatocellular carcinoma, however the APM remains to be examined (87, 88).

Alternatively to HDACi there were two HDACs, 5 and 10, which showed an enhanced activity upon treatment with P02-113 and P03-97-1. These are most likely the HDACs candidates showing an increase in activity in the generalized HDAC class I/II assay preformed on the A9 cell line. This is a unique finding as HDACs are currently viewed as being overactive in cancer to decrease the expression of cancer preventing genes. However, reductions in activity of both HDACs 5 and 10 have been implemented in advanced stages of lung cancer and are correlated with poor outcome (89, 90). Interestingly previous studies that have down regulated HDAC5 using siRNA found that there was a pro-angiogenic effect due to increased endothelial cell migration, sprouting, and tube formation (91). As for HDAC10, there have been significantly more research done is relation to its activity in cancer. Decreases in HDAC10 activity has been correlated with more aggressive malignancies in B cell and gastric cancers and has been correlated with metastasis in gastric cancer and squamous cell carcinomas (92-94). A mechanism has also been demonstrated for HDAC10 involvement with metastasis as it is known to suppress matrix metalloproteases 2 and 9 that are critical for cancer cell invasion and metastasis (92). Therefore future work to establish if HDAC5 and HDAC10 are crucial to the regulation of the APM will be fundamental to understand if P02-113 and P03-97-1 exhibit up-regulation through the enhancement of HDACs 5 and 10. Down-regulation of these enzymes in the primary TC-1 cell lines will help establish if this is a contributing mechanism.

### BIBLIOGRAPHY

1. Laird, P. W., & Jaenisch, R. Annual review of genetics 30(1), 441-464 (1996).
2. Bishop, J. M. Science 235(4786), 305-311(1987).
3. Nguyen, D. X., & Massagué, J. Nature Reviews Genetics 8(5), 341-352 (2007).
4. Jones, P. A., & Baylin, S. B. Nature reviews genetics 3(6), 415-428 (2002).
5. Balmain, A., Gray, J., & Ponder, B. Nature genetics 33, 238-244 (2003).
6. Esteller, M. New England Journal of Medicine 358(11), 1148-1159 (2008).
7. Escors, D. New journal of science (2014).
8. Seliger, B., et al., S. Semin Cancer Biol 12, 3-13 (2002).
9. Setiadi, A. Francesca, et al. Cancer research 68(23) 9601-9607 (2008).
10. Setiadi, A. F. et al. Molecular and cellular biology 27(22), 7886-7894 (2007).
11. Setiadi, A. F et al. Cancer research 65(16), 7485-7492 (2005).
12. 2016. Cancer statistics at a glance. Canadian Cancer Society.
13. Tomlinson, I., & Bodmer, W. Nature medicine, 5(1) (1999).
14. Mehlen, P., & Puisieux, A. Nature Reviews Cancer, 6(6), 449-458 (2006).
15. 2016. Overveiw of Metastasis. Cancerquest.
16. Nguyen, D. X., & Massagué, Nature Reviews Genetics, 8(5), 341-352 (2007).
17. Jones, P. A., & Baylin, S. B. Nature reviews genetics 3(6), 415-428 (2002).
18. Kim, R., Emi, M., & Tanabe, K. Immunology, 121(1), 1-14(2007).
19. Reiman, J. M., et al. Seminars in cancer biology, 17 (4), 275-287 (2007)
20. Thompson, A. E. The immune system. Jama 313(16), 1686-1686 (2015).
21. Shreffler, D. C., & David, C. S. Advances in immunology 20, 125-195 (1975).
22. Cromme F V et al. 1994. British journal of cancer, 69(6):1176.
23. Cabrera, T. et al. Hum Immunol 61, 499-506 (2000).
24. Seliger, B., et al. Immunology today, 21(9), 455-464 (2000).
25. Garcia-Lora, A., et al. Journal of cellular physiology, 195(3), 346-355 (2003).
26. Seliger, Barbara, et al. Cancer research, 56(8), 1756-1760 (1996).
27. Cromme, F. V., et al. British journal of cancer, 69(6), 1176 (1994).
28. Johnsen, A., et al. Cancer research, 58(16), 3660-3667 (1998).
29. Vigushin, D. M., et al. Clinical Cancer Research, 7(4), 971-976 (2001).De
30. Visser, K. E., et al. Nature reviews cancer 6(1), 24-37 (2006).
31. Schnare, M., et al. Nature immunology, 2(10), 947-950 (2001).
32. Iwasaki, A., & Medzhitov, R. Science, 327(5963), 291-295 (2010).
33. Roche, P. A., & Furuta, K. Nature Reviews Immunology 15(4), 203-216.
34. Rock, K. L., & Goldberg, A. L. Annual review of immunology 17(1), 739-779 (1999).
35. Concha-Benavente, F., Srivastava, R., Ferrone, S., & Ferris, R. L. 2016. Oral Oncology.
36. Agrawal, Suraksha, and Moitryee Chaterjee Kishore. Journal of hematotherapy & stem cell research 9.6 (2000): 795-812.
37. Cock-Rada, A., & Weitzman, J. B. Biology of the Cell, 105(2), 73-90 (2013).
38. Campoli, M., & Ferrone, S. Oncogene, 27(45), 5869-5885 (2008).
39. Feinberg, A. P., & Tycko, B. Nature Reviews Cancer, 4(2), 143-153 (2004).
40. Dawson, M. A., & Kouzarides, T Cell, 150(1), 12-27 (2012).
41. Ma, X., Ezzeldin, H. H., & Diasio, R. B. Drugs, 69(14), 1911-1934 (2009).
42. Kroesen, M., et al. Oncotarget, 5(16), 6558-72, (2014).
43. Rodriguez-Paredes, M., & Esteller, M. Nature medicine, 330-339 (2011).
44. Lai, P. K., & Roy, J. Current medicinal chemistry, 11(11), 1451-1460 (2004).
45. Thompson, L. U., et al. Carcinogenesis, 17(6), 1373-1376 (1996).
46. Abdullaev, F. I. Experimental biology and medicine, 227(1), 20-25 (2002).
47. Busquets, S., et al. Cancer letters, 167(1), 33-38 (2001).
48. Greenwell, M., & Rahman, P. K. S. M. International journal of pharmaceutical sciences and research, 6(10), 4103 (2015).
49. Simmons, T. L., et al. Molecular Cancer Therapeutics, 4(2), 333-342
50. Gomes, N. G et al. Marine drugs, 14(5), 98 (2005).
51. Rodrigo, G., et al. Fitoterapia, 81(7), 762-76 (2010).
52. Gaspar, H., et al. Marine Drugs, 2(1), 8-13 (2004).
53. El Sayed, K. A., et al. Journal of natural products, 65(11), 1547-1553 (2002).
54. Behery, F. A., et al. Med Chem Comm, 3(10), 1309-1315 (2012).
55. Fusetani, N., et al. Cellular and Molecular Life Sciences, 43(11), 1234-1235 (1987).
56. Wright, A. E., et al. Journal of Natural Products, 50(5), 976-978 (1987).
57. Behery, F. A., et al. Med Chem Comm, 3(10), 1309-1315 (2012).
58. Gaspar, H., et al. Bioactive Semisynthetic Dervatives of (S)-(+)-Curcuphenol (2008).
59. Cichewicz, R. H., et al. Bioorganic & medicinal chemistry, 13 (19), 5600-5612 (2005).
60. Tsukamoto, S., et al. Antifouling terpenes and steroids against barnacle larvae from marine sponges (1997).
61. Chung, H. M., et al. Marine drugs, 10(5), 1169-1179 (2012).
62. Takamatsu, S., et al. Journal of Natural Products, 66(5), 605-608 (2003).
63. Hertiani, T., et al Indonesian Journal of Pharmacy, 78-85 (2008)..
64. Frame, M. C. Reviews on Cancer, 1602(2), 114-130 (2002).
65. Valdivia, A. C., et al Revista Boliviana de Quimica, 26(2), 77-82 (2009).
66. Yonezawa, T et al (2004). Biochemical and biophysical research communications, 314(3), 805-809.
67. Hardy, S., et al Journal of Biological Chemistry, 280(14), 13285-13291(2005).
68. Tomita, T., et al. Diabetologia, 49(5), 962-968 (2006).
69. Chung, H. M et al. Marine drugs, 10(5), 1169-1179 (2012).
70. Chen, C. Y., et al. Journal of natural products, 62(4), 573-576 (1999).
71. Valdivia, A. C., et al. Revista Boliviana de Química, 26(2), 77-82 (2009).
72. Manning, Jasper, et al. Immunology 123, no. 2 (2008): 218-227.
73. Šmahel, M., et al. Immunisation with modified HPV16 E7 genes against mouse oncogenic TC-1 cell sublines with downregulated expression of MHC class I molecules (2003).
74. Indrová, M., et al. International journal of oncology, 28(1), 253 (2006).
75. Cromme, F. V., et al. The Journal of experimental medicine, 179 (1) 335-340 (1994).
76. Maeurer, Markus J., et al. Journal of Clinical Investigation 98 (7), 1233 (1996).
77. Creier, Carola, C. C. Investigation of extract influence on antigen presentation in Nef-expressing dendritic cells and cancer cells. MS thesis. University of Applied Sciences Mannheim, 2011
78. Sanderson, L., Taylor, G. W., Aboagye, E. O., Alao, J. P., Latigo, J. R., Coombes, R. C., & Vigushin, D. M. Drug Metabolism and Disposition, 32(10), 1132-1138 (2004).
79. Sakkal, S., et al. Current medicinal chemistry, 23(7), 650-666 (2016).
80. Venkatesan, R., et al. Cancer Treatment Communications, 4, 55-58 (2015).
81. Astigiano, Simonetta, et al. Neoplasia 7, 4, 390-396 (2005).
82. Khalil, D. N., Smith, E. L., Brentjens, R. J., & Wolchok, J. D. The future of cancer treatment: immunomodulation, CARs and combination immunotherapy. Nature Reviews Clinical Oncology, 13(5), 273-290 (2016).
83. Abken, H., et al. Frontiers in immunology, 4, 371 (2013).
84. Beher, D., et al. Chemical biology & drug design, 74(6), 619-624 (2009).
85. Chakrabarti, et al. Trends in pharmacological sciences, 36(7), 481-492 (2015).
86. Gao, J., et al. Biochemical and biophysical research communications, 379(1), 1-5 (2009).
87. Wu, Jian, et al. Digestive diseases and sciences, 58 (12), 3545-3553 (2013).
88. Vannini, A., et al. Proceedings of the National Academy of Sciences of the United States of America, 101(42), 15064-15069 (2004).
89. Yao, H., & Rahman, I. Current opinion in pharmacology, 9(4), 375-383 (2009).
90. Osada, H., et al. International journal of cancer, 112(1), 26-32(2004).
91. Urbich, et al. Blood, 113(22), 5669-5679 (2009).
92. Song, C., et al. Journal of Biological Chemistry, 288(39), 28021-28033(2013).
93. Jin, Z., et al. Int J Clin Exp Pathol, 7(9), 5872-5879(2014).
94. Powers, J et al. B Cell Malignancies. Histone Deacetylases: Methods and Protocols, 129-145(2016).

### Example 2

Metastatic colonization is movement of cancer from a primary to a secondary site, and involves the survival and proliferation of disseminated cells (1). Oftentimes, metastasizing cancers acquire the ability to evade the immune system, particularly though the downregulation of antigen presentation machinery (APM) (2). Our lab focuses on APM related to tumor antigen presentation on major histocompatibility complexes (MHC) to CD8+ cytolytic T-lymphocytes (CTL). The APM consists of several components, including the proteasome, the transporters of antigen 1 & 2 (TAP1, TAP2), and MHC class I. The disruption of any one of these components will result in faulty presentation of endogenous tumor antigens on nascent MHC class I, and as such, improper recognition of the tumor by CD8+ T-cells.

The APM of relevance to our cancer work presents internal endogenous peptides to a CTL (3). Multi-catalytic proteasome complexes and cytosolic proteases degrade cytosolic internal peptides, and TAP-1 & 2 bring the product peptides to the endoplasmic reticulum. The peptides are loaded onto MHC class I before being presented on the cell surface to cytolytic T-lymphocytes (3). CTL are critical for immunosurveillance against tumors; abnormalities in expression or function of the APM may cause downregulation of cell surface expression of the MHC class I antigens that CTLs require (3). Cytokines, such as IL-33, may cause the upregulation of the APM. Improperly functioning machinery appears in up to 90% of metastatic cancers (4). Many tumors, including lung carcinoma, lack TAP-13. TAP-1 downregulation is thought to allow for tumor evasion of the immune system.

The cell lines being compared in these experiments are primary TC1 tumour line and the metastatic A9 tumor line. The primary TC1 tumor cell line was developed by the transformation of murine primary lung cells with the human papilloma virus type16 E6 and E7 oncogenes and activated H-ras (cell-division regulating GTP-ase). These cells have high TAP-1 and MHC class I levels (5). The metastatic A9 tumor cells are the metastatic clones derived from the primary TC1 tumor; these cells are capable of metastasis when injected subcutaneously into mice, not just when injected into blood (6). The metastatic A9 tumor has downregulated MHC class-I expression and APM components (5).

Previously our lab observed a decrease in global acetylation from primary tumor cell lines to metastatic tumor cell lines (5). We also demonstrated a decrease in TAP-1 expression between primary and metastatic tumor cell lines, specifically primary TC1 and metastatic A9, which was caused by chromatin remodelling. This correlated with a decrease in MHC class I. Furthermore, we have shown that Trichostatin A (TSA), a known histone deacetylase inhibitor (HDACi), is effective in restoring the MHC-I on A9 cells.

Determining the mechanism that causes reduction of immune surveillance through downregulation of APM is an important step towards using these compounds to enhance immune recognition of tumors. It will also provide us with protein targets that will help restore the original APM function, and possibly lead to therapeutics to minimize cancer metastasis. This knowledge could be considered for not only cancer therapy, but a greater understanding of immunosurveillance will aid in vaccine development.

A functional screen was established to identify products that increase the expression of TAP-1 and MHC class I in metastatic tumors thereby reversing the immune escape phenotype of metastatic cells. We identified curcuphenol as being a molecule that enhances the expression of MHC-I on A9 cells, with low cellular toxicity. Here we present evidence of cellular pathways through which this induction may take place.

Research Methods

Cell lines primarily being used: TC1 and A9

Methods primarily used: Fluorescence Activated Cell Sorting (FACS), western blot, RealTime-PCR (RT-PCR), Proteome Profiler Mouse Cytokine Array Kit (Panel A).

Summary of Results

Upon addition of 0.014 mg/ml (0.064 umol) of Curcuphenol, there was a change in the cytokine production of A9 cells, relative to that of cells treated with only vehicle DMSO. This treatment was done in 2 mL of DMEM media, at an initial seeding density of 1×10⁶ A9 cells, and treatment with Curcuphenol in DMSO vehicle or DMSO vehicle was done 24 hours after seeding A9 cells. A9 cells are murine metastatic epithelial carcinoma cells. Cytokine change was seen using the Proteome Profiler Mouse XL cytokine array (R&D Systems, ARY028).

Decrease in the cytokine production from A9 cells upon treatment with Curcuphenol: WISP-1/CCN4; IGFBP-3; Amphiregulin; IGFBP-6; IGFBP-2; CD160; MMP-2; CCL22/MDC; IL-12 p40; IL-6; Pentraxin 2/SAP; TNF-alpha; Chemerin; CCL2/JE/MCP-1; CXCL10/IP-10; CCL5/RANTES; CCL6/C10; CCL11/Eotaxin; CXCL9/MIG; CXCL11/I-TAC; E-Selectin/CD62-E; P-Selectin/CD62P; CCL17/TARC; IL-11; Angiopoietin-1; IL-10; Adiponectin/Acrp30; Endostatin; M-CSF; IL-7; MMP-3; Flt-3 Ligand; Pref-1/DLK-1/FA1 Increase in the cytokine production from A9 cells upon treatment with Curcuphenol: Proliferin; DGF-BB; Gas 6; GDF-15; Pentraxin 3/TSG-14; IL-33; IL-1alpha/IL-1F1; Myeloperoxidase; CXCL16; IL-1ra/IL-1F3; IL-15; IL-1beta/IL-1F2; IL-28A/B; IFN-gamma; CD40/TNFRSF5; CCL12/MCP-5; CCL19/MIP-3beta; CXCL13/BLC/BCA-1; LIX; CX3CL1/Fractalkine; Serpin F1/PEDF; Angiopoietin-like 3; Angiopoietin-2; IL-13; Coagulation Factor III/Tissue Factor; FGF-21; VEGF; Serpin E1/PAI-1; Osteopontin (OPN); Cystatin C; TIM-1/KIM-1/HAVCR; G-CSF

No change in the cytokine production from A9 cells upon treatment with Curcuphenol: VCAM-1/CD106; Proprotein Convertase 9/PCSK9; Leptin; Periostin/OSF-2

### REFERENCES

1. Smulewitz, R., Taylor, J. & Rinker-Schaffer, C. Encyclopeida of Cancer: Chapter “Metastatic Colonization”, 3rd edn. Springer, 2012.
2. Alimonti, J. et al. Nat Biotechnol 18, 515-520. (2000).
3. Seliger, B., et al. Immunol Today 21, 455-464 (2000).
4. Haworth, K. B. et al. Pediatr Blood Cancer 62, 571-576 (2015).
5. Setiadi, A. F. et al. Molecular and cellular biology 27, 7886-7894 (2007).
6. Saranchova, I. et al. D Sci Rep 6, 30555 (2016).

### Example 3

### Abstract

Cancer evasion of the immune system can be initiated by the down-regulation of the cellular antigen processing machinery (APM), through genetic and epigenetic events. Without these essential components, metastatic cancers subvert host immune surveillance and are thus resistant to many immunotherapies that evoke the adaptive immunity to eradicate tumours. The marine environment dominates all other natural environments in its biodiversity making it an important resource for bioactive natural product discovery. (+)-Curcuphenol, a sesquiterpene phenol, was identified from a chemical library made from marine invertebrate extracts using a novel high-throughput cell-based assay to identify compounds that induce the expression of APM components within metastatic prostate and lung carcinomas. Synthetic non-chiral, water soluble curcuphenol analogs were prepared by informed design and found to possess novel and unprecedented histone deacetylase enhancing (HDACe) activity that induces APM component gene expression, including MHC I and Tap1, in both metastatic prostate and lung carcinomas. Treatment of metastatic lung carcinomas-bearing mice with these compounds resulted in significant reduction in the mean tumour volume and increase in cytotoxic T-cell tumour infiltration. The discovery of novel natural products and their improved analogs, that enhance immune responses against metastatic tumours by reversing immune-editing and escape provides rationale for the development of naturally products as therapeutic candidates for harnessing the power of the immune system to recognize and destroy metastatic cancers.

### INTRODUCTION

Understanding the mechanisms that promote a primary cancer to advance to a metastatic derivative is of great concern as metastatic cancers account for 90% of all cancer deaths¹. The cellular immune system plays an essential role in reducing cancer progression through recognition of cancer cells via the antigen processing pathway (APM). In the APM, cellular peptides are presented via the major histocompatibility class I molecules (MHC-I), located on the surface of all nucleated cells in the body, to the cytotoxic T lymphocytes (CTLs) of the immune system. In humans, the MHC-I molecules are referred to as human leukocyte antigen (HLA). To generate the peptides, endogenous proteins are degraded by the proteasome in the cytosol before being transported into the endoplasmic reticulum (ER) by the transporters associated with antigen processing 1 and 2 (TAP-1/2). In the ER, the peptides are loaded onto the MHC-I molecules before being transported to the cell surface. Upon interaction of the CTLs with the MHC-I peptides complexes on the cell surface, the CTLs are able to distinguish between normal, cancerous, or pathogen-infected cells. Following this interaction, an appropriate immune response can be initiated, which often leads to the destruction of the cancerous or pathogen-infected cells¹⁻³.

During a cancer's evolution, there are several genetic and epigenetic alterations, some of which allow the cancer to become genetically unstable and subsequently metastatic. These genetic changes are referred to as a metastatic signature. The selective pressure of immune-surveillance on genetically unstable tumour populations may yield tumours that have lost expression of antigen processing machinery (APM) components, often resulting in reduced assembly of functional major histocompatibility complex (MHC or HLA) molecules. The mechanisms underlying immunoevasion of the adaptive immune system was described by Alimonti et al.⁴ and termed immune-subversion or immune-escape, and subsequently confirmed by Shankaran et al.⁵ and termed immune-editing. A common metastatic signature seen in several forms of cancer is one that allows immunoevasion. Escape from immune recognition is the result of a number of mechanisms that operate either exclusively or in any combination with the following: tumour-induced T-cell anergy, absence or low expression of MHC-I molecules, and/or defective MHC-I antigen presentation machinery (APM)^{4,5}. Alteration in the expression of surface MHC-I molecules is an important tumour escape mechanism since MHC-I antigens are required for antigen presentation to CTLs and the regulation of natural killer cells. In some carcinomas, the frequency of MHC-I loss approaches 100%^{6,7}. Since entry of processed peptides into the ER via TAP-1/2 is required for the construction of MHC-I-peptide complexes, the loss of TAP-1/2 greatly contributes to a functional defect in the APM⁸. These cellular phenotypic changes associated with malignant transformation ultimately disable the cell's ability to present peptides on the cell surface, thus allowing malignant cells to evade immune surveillance⁹⁻¹¹. Tumour cells that have defects in the APM appear to have a selective advantage compared to other tumour cells that retain a functional APM, conferring on them a greater metastatic potential. Several types of cancer, including breast cancer^{12,13} renal carcinoma¹⁴, melanoma^{15,16} colorectal carcinoma¹⁷, head and neck squamous cell cancer¹⁸, cervical cancer¹⁹, and finally prostate carcinoma show a clear correlation between HLA down-regulation and poor prognosis²⁰⁻²². The increasing frequency of immune-escape tumour variants in many forms of metastatic cancers is a predictor of disease progression as well as patient outcome. However, few attempts have been made to directly overcome the APM deficits in immune-escape tumour variants as a therapeutic modality to treat metastatic disease. It has been previously demonstrated that by restoring TAP-1 expression in metastatic cells it is possible to restore APM and the CTL recognition of MHC-I molecules in murine carcinomas^{2, 8, 23-26} Additionally, it was shown that APM deficiency can be restored in vitro and in vivo by complementation of TAP expression²⁸, by either transformation with virus vectors containing the TAP gene^{23, 27} or with immune enhancers²⁸. Intriguingly, in a previous study it has been found the TAP-1 deficiency was not regulated by defects or mutations in the TAP-1 gene, but it was epigenetically regulated in two separate cell lines²⁹ and could be restored by treatment with histone deacetylase inhibitors (HDACi), such as trichostatin-A (TSA)²⁸.

Previous studies have demonstrated that although TSA, an HDACi, has been shown to promote differentiation, cell cycle arrest and apoptosis in tumour cells³⁰, TSA is not effective in decreasing tumour growth in TSA-treated Rag-1^{-/-} mice²⁸, which lack functional lymphocytes. These findings strongly suggest that in TSA-treated animals, the immune recognition of tumours is increased and that the TSA effect is mediated by the adaptive immune response in vivo²⁸. Although TSA has been shown to confer anti-cancer effects in vitro and in vivo³¹⁻³³, cancer treatments using natural HDAC inhibitors, such as TSA, depudecin, trapoxins, apicidins, sodium butyrate, and phenyl butyrate are inefficient due to their instability and low retention in vivo³⁴. This limitation may be overcome by the development of non-toxic compounds that possess new activities, high stability in vivo, and improved efficacy to induce immune recognition of tumours.

The natural products paclitaxel, vincristine, doxorubicin, and bleomycin are among the most important anticancer drugs in clinical use³⁵ and it has been estimated that between the 1940s and 2014, roughly 50% of all new FDA approved anticancer drugs were either natural products or derived from natural products³⁶. Marine organisms represent a highly biodiverse but relatively unexplored resource for the discovery of new natural product anticancer drug leads³⁷. Realization of the promise of this resource is illustrated by the clinically approved anticancer drugs Ara-C, Adcetris, Yondelis and Halavan, which are all based on natural products isolated from marine invertebrates. The marine invertebrate extract collection screened in this study has been a rich source of novel natural product chemical biology tools and drug leads³⁸⁻⁴³ and, therefore, it was selected as an excellent resource for discovery of new compounds that may overcome immunoevasion.

Here, we describe the discovery of compounds from marine extracts with previously undescribed HDACe activity with the potential to reduce immune escape and reduce the growth of metastatic tumours.

Results

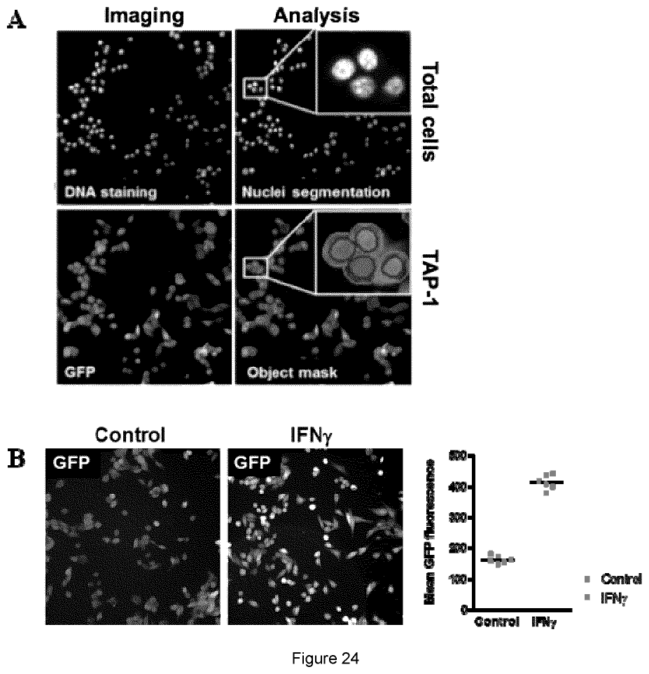

Identification of marine natural product extracts with the ability to promote up-regulation of TAP-1 and MHC-4 expression in cancer cells: A high-throughput cell-based screen was used for identification of candidate compounds that increase the expression of TAP-1 in the LMD murine metastatic prostate cell line. To assess the induction of TAP-1 expression we used the LMD:TAP-1 cell line, which was transfected with a vector containing EGFP under the TAP-1 promoter⁴⁴. The Cellomics™ Arrayscan VTI automated fluorescence imager was used to determine the cell numbers based on DNA staining and the average GFP fluorescence intensity which correlates to the levels of TAP-1 induction (FIG. 24A). The vehicle solution of 1% DMSO in cell culture medium was used as the negative control and IFN-γ (10 ng/ml in 1% DMSO), a known inducer of TAP-1 expression, was used as the positive control (FIG. 24B).

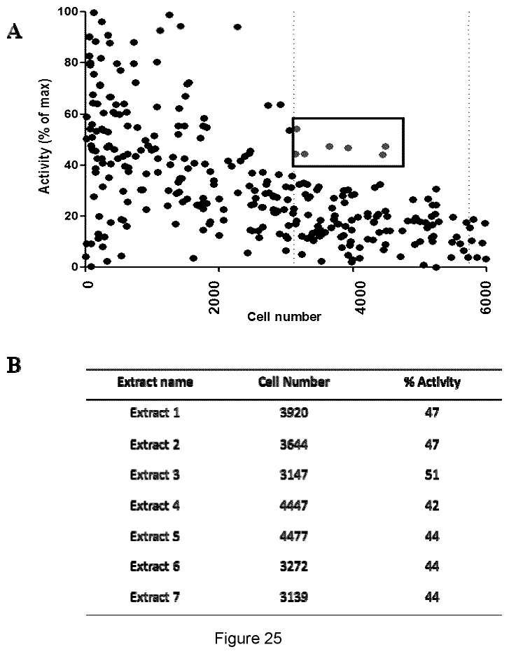

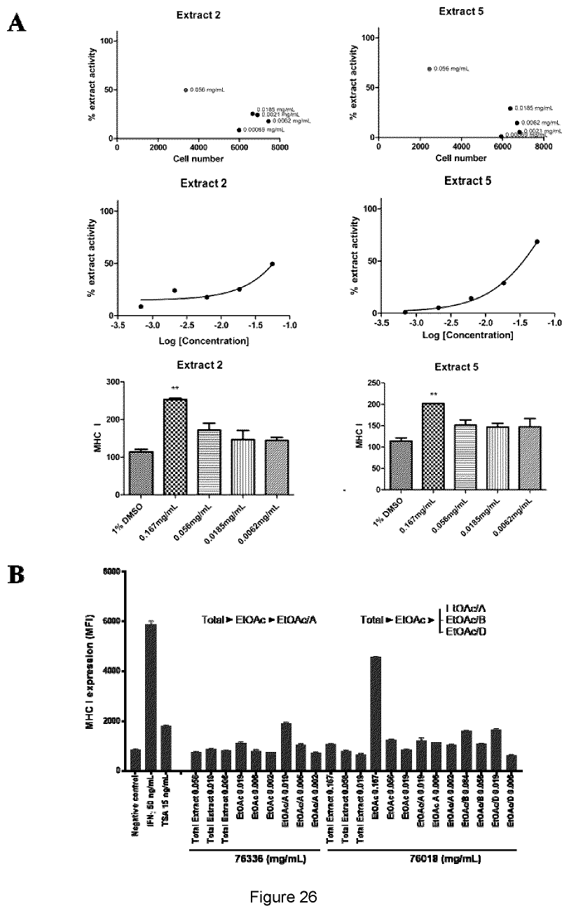

A library, with a total of 480 marine sponge extracts estimated to contain thousands of natural products, was screened using the high-throughput cell-based assay to assess the induction of TAP-1 expression in the LMD: TAP-1 cell line. From this screen, seven extracts were selected based on significant TAP-1 induction (>40% activity when compared to the positive control) and low cell cytotoxicity (within 1 standard deviation of average cell density of vehicle alone) (FIG. 25). Upon retesting the seven extracts using varying concentrations, two of the extracts, Extract 2 (76018 and Extract 5 (76336) were highly replicable and titratable (FIG. 26A, top and middle). Extracts 2 and 5 were further tested for their ability to induce MHC-I expression at the cell surface in the LMD and A9 cells, 48 hours following treatment using flow cytometry. Both extracts showed a significant increase in cell surface MHC-I expression (FIG. 26A, bottom), making them strong candidates for therapeutic agents in immunoevasive cancers.

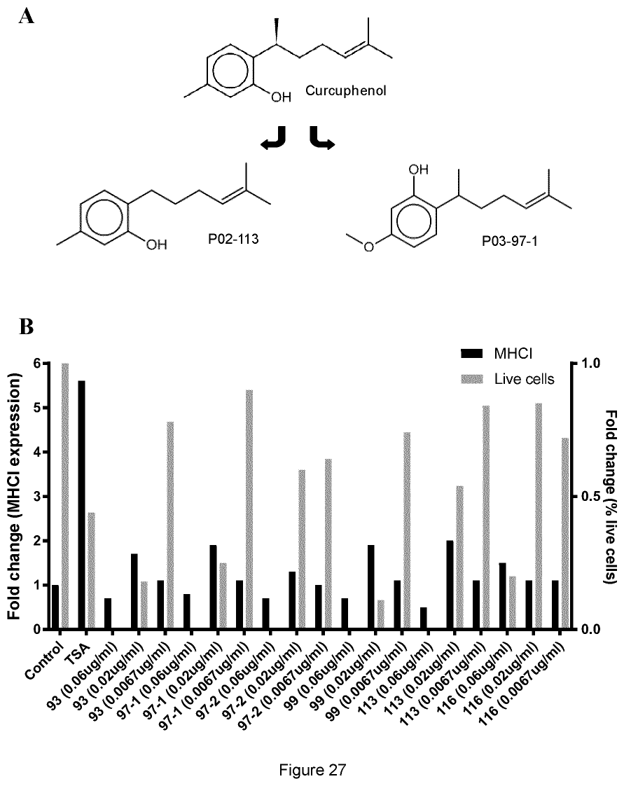

To identify the active biological components of the extracts, Extracts 2 and 5 were subjected to assay-guided fractionation using solvent/solvent (water/ethyl acetate) partioning, Sephadex LH20 size separation chromatography, and HPLC. The purified fractions from Extracts 2 and 5 were tested alongside the whole extracts for their ability to induce MHC-I expression. One fraction, comprised of the ethyl acetate soluble materials from Extract 2 (76018: Halichondria sp) showed significant increase in MHC-I expression compared to all other fractions tested (FIG. 26B). Further assay-guided fractionation of the 76018 ethyl acetate soluble material gave a pure active natural product that was identified by NMR and MS analysis to be curcuphenol (FIG. 27A), a compound not only found in sea sponges but also in turmeric, a common spice used in Asian and Indian cooking.

Isolation, identification and synthesis of curcuphenol and its synthetic analogs: Racemic curcuphenol was synthesized after curcuphenol was identified as a potential therapeutic agent. A series of lower C Log P and achiral analogues that had structural modifications on the phenol ring and on the carbon tail were also synthesized and assessed for their ability to induce the MHC-I in vitro. From this small synthetic library, two analogues, P02-113 and P03-97-1 (FIG. 27A), showed the greatest consistent induction of MHC-I expression at the cell surface 48 hours after treatment when measured by flow cytometry, while maintaining low cytotoxicity (FIG. 27B).

In vivo effects of PC-02-113 and P03-97-1 in tumour-bearing mouse model: P02-113 and P03-97-1 curcuphenol analogs were assessed in vivo for the maximal tolerated dose based on the maximum solubility of the compounds in 1% DMSO. Three doses were tested for each compound (1.0 mg/kg, 3.5 mg/kg, and 5.2 mg/kg), and both compounds were well tolerated in mice at the highest dose with no adverse drug effects, as assessed by necropsy 2 weeks after i.p. administration.

To find an optimal dosing schedule for in vivo studies, the compounds were also analyzed for their pharmacokinetic properties in mouse plasma. Three time points were used: 5 minutes, 10 minutes and 1 hour, with three mice per group for both compounds. From the pharmacokinetic analysis, the half-life of both compounds in mouse plasma was roughly one hour. Due to the short half-life of the compounds, it was decided that everyday treatment would be necessary during the in vivo studies.

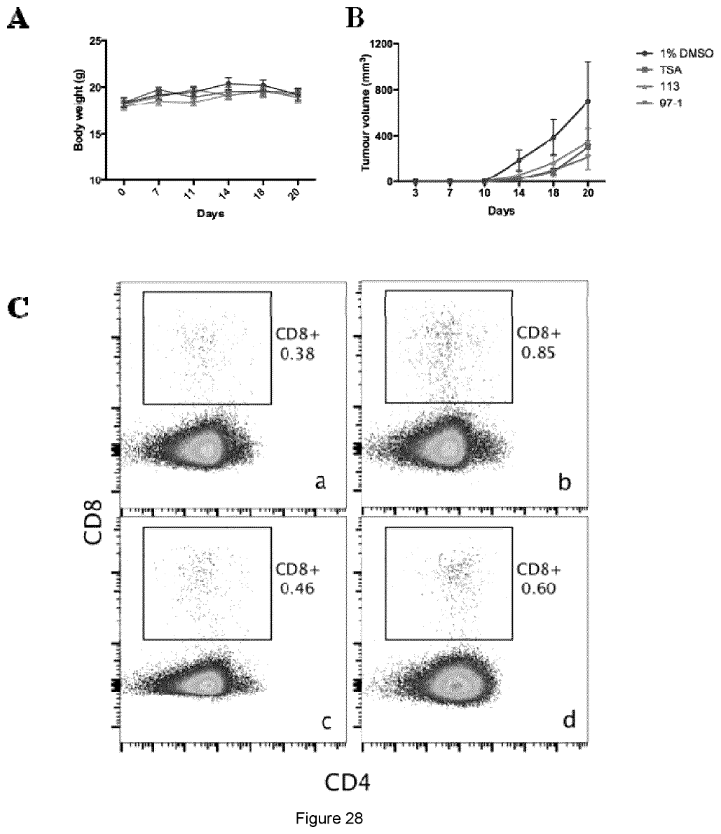

Mice were inoculated subcutaneously in the right flank with 5×10⁴ A9 metastatic tumour cells, and tumours were allowed to grow for 7 days. After 7 days, mice were treated everyday for 12 days with either TSA (positive control, 500 μg/kg), 1% DMSO (negative control), or one of the two test compounds, P02-113 or P03-97-1 (at 5.2 mg/kg). Body weights were measured every 2-4 days and there were no significant changes observed in body weight in any of the 4 groups (FIG. 28A). The tumours were measured in all groups 3 times a week; all mice that did not develop tumours during the study were removed from tumour volume analysis. After treatment of mice for 12 days, there was a statistically significant reduction of tumour volume between treated groups (P02-113 and P03-97-1) and the untreated group (1% DMSO), as determined using a one tailed t-test (p<0.0001). The tumours were also processed and analyzed by flow cytometry for the induction of TIL (CD8+ CTLs) in all mice that developed tumours. There was an increase in the CTL infiltration in the P03-97-1 and TSA groups as compared to the untreated control. Interestingly, the P02-113 treatment group, there was no significant change in CTL infiltration, and the tumours in this group were slightly larger than the tumour volumes seen in either the P03-97-1 and TSA groups. The numbers of CD4+ cells infiltrating tumours did not appear to increase over that of the negative control tumours, although these numbers were assessed at the end of the trial, and not determined throughout. However, both compounds, P02-113 and P03-97-1, showed great anti-cancer therapeutic potential in vitro, as well as in vivo. The ability of the compounds to induce both TAP-1 and MHC-I makes them great candidates for human trials, as loss of expression of these proteins is a phenotype that is seen across several forms of cancers. Future studies will be needed to gain a full understanding of the anti-tumour mechanism(s) of these compounds.

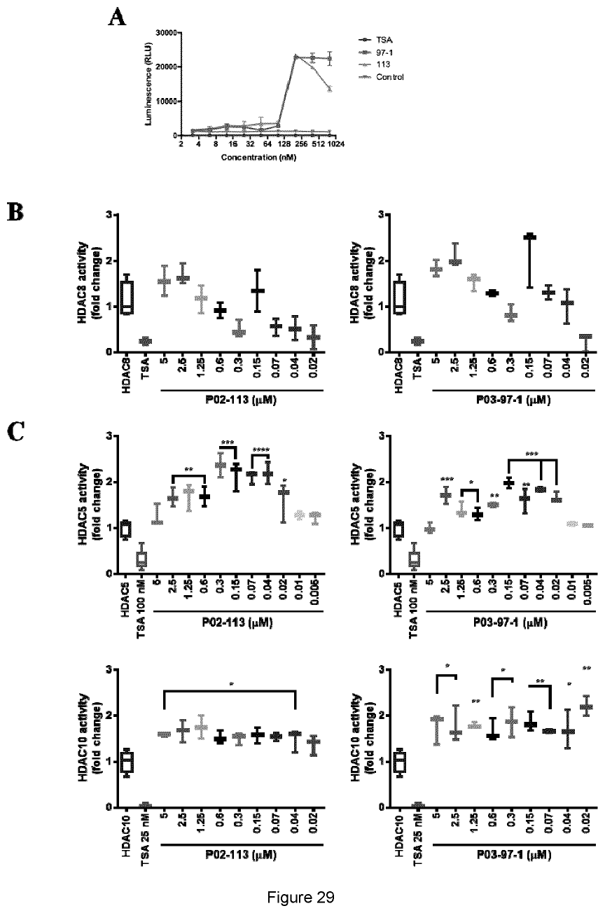

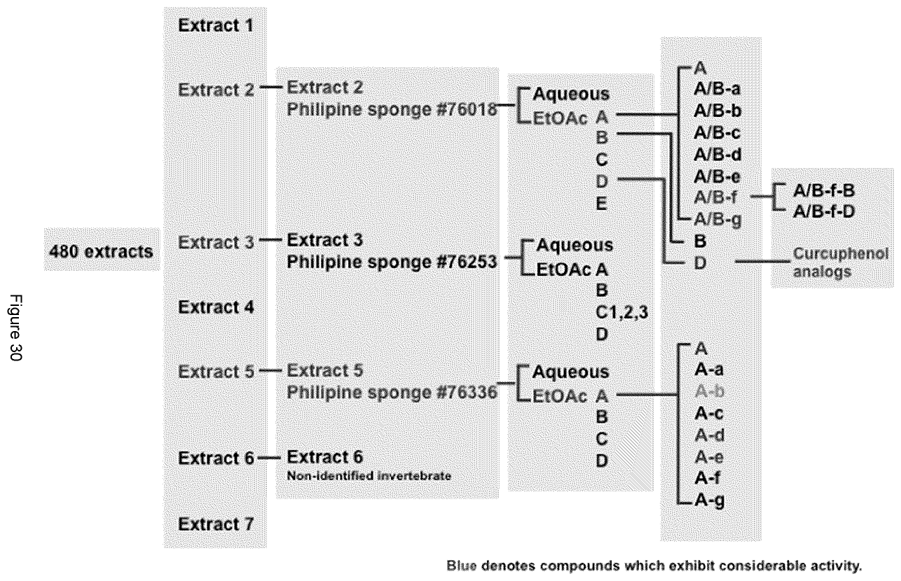

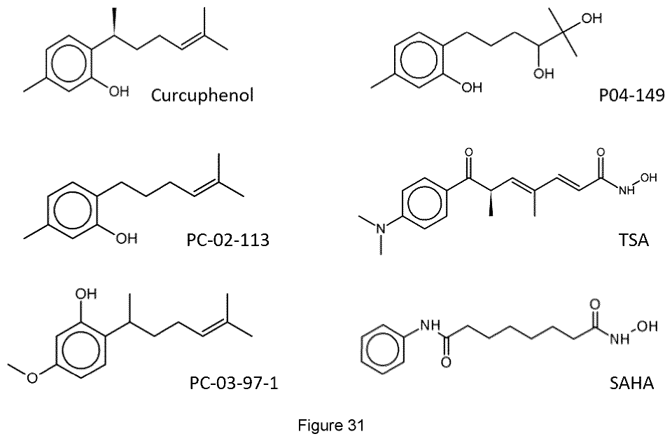

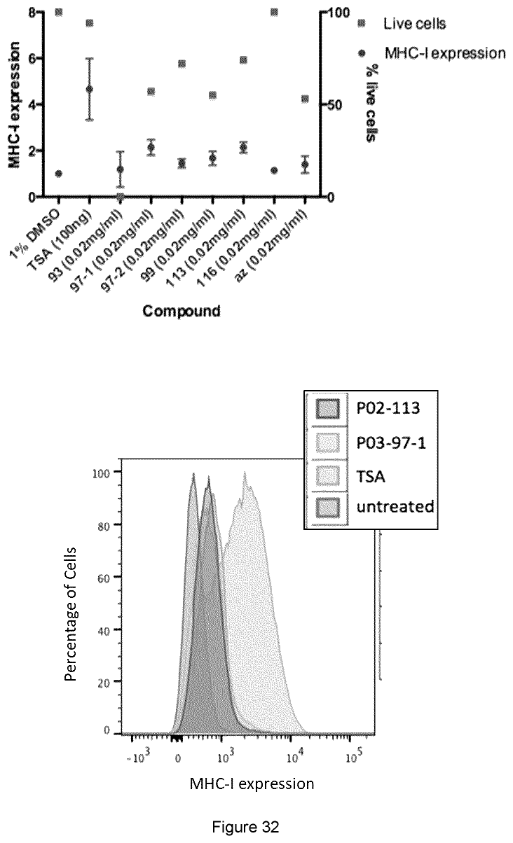

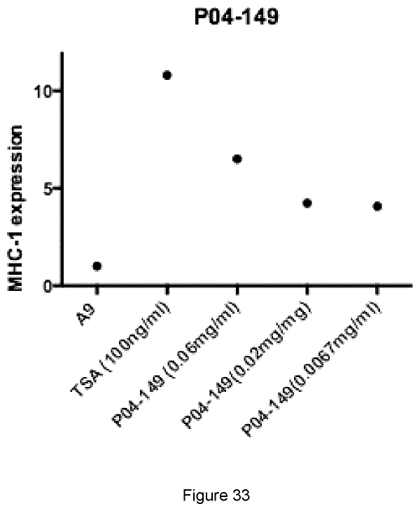

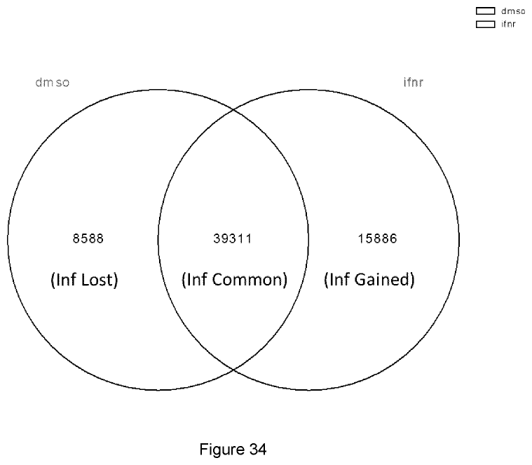

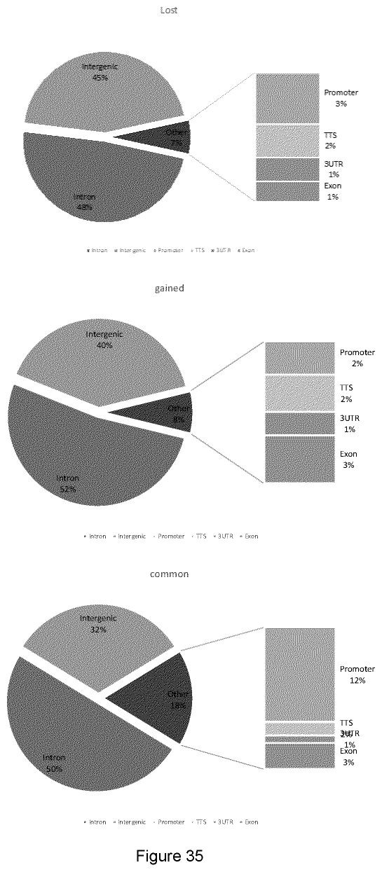

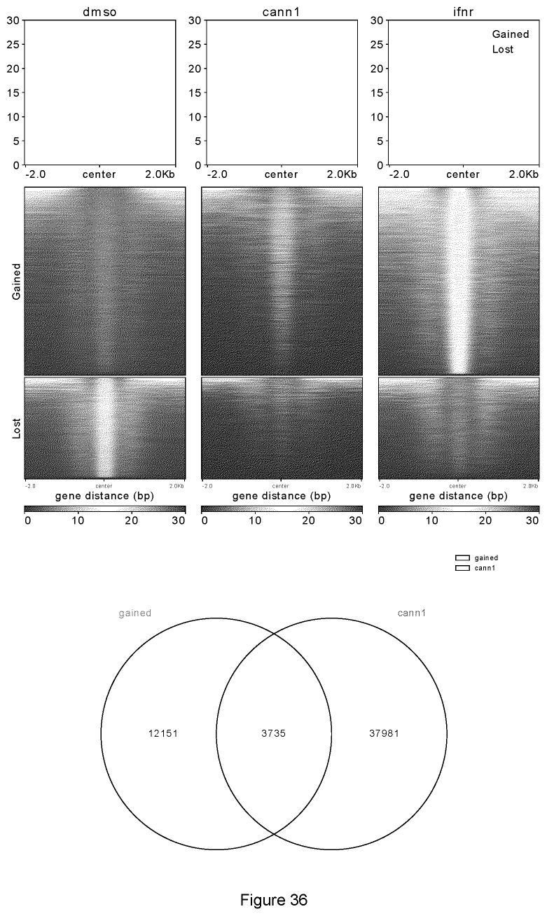

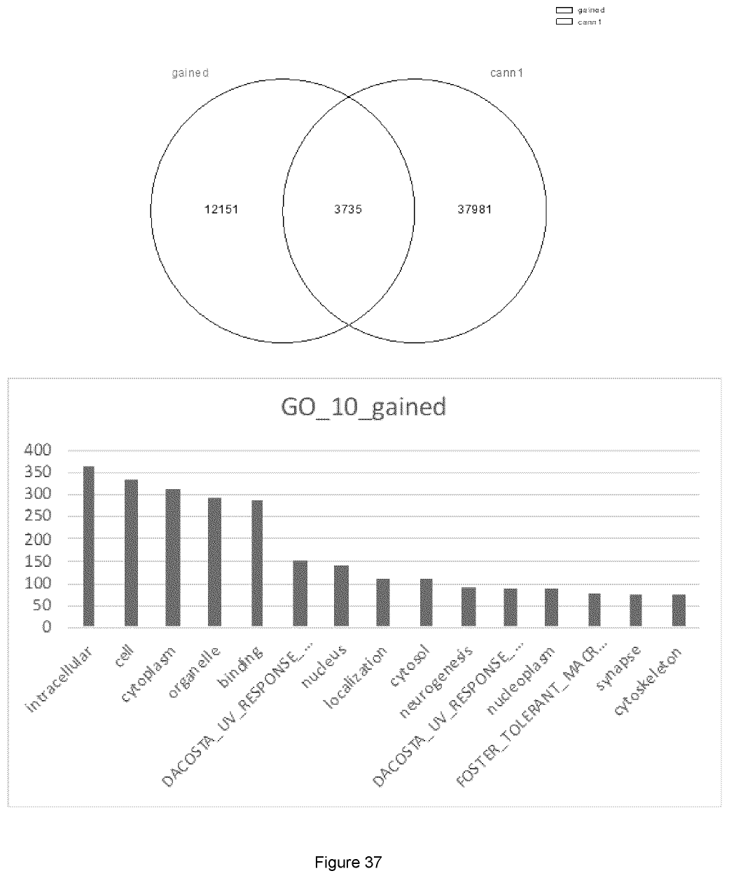

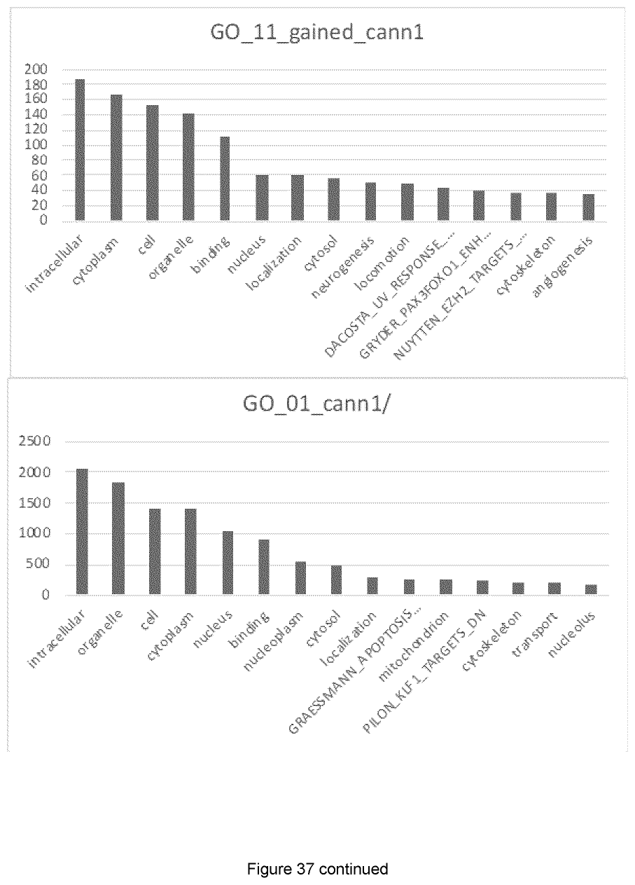

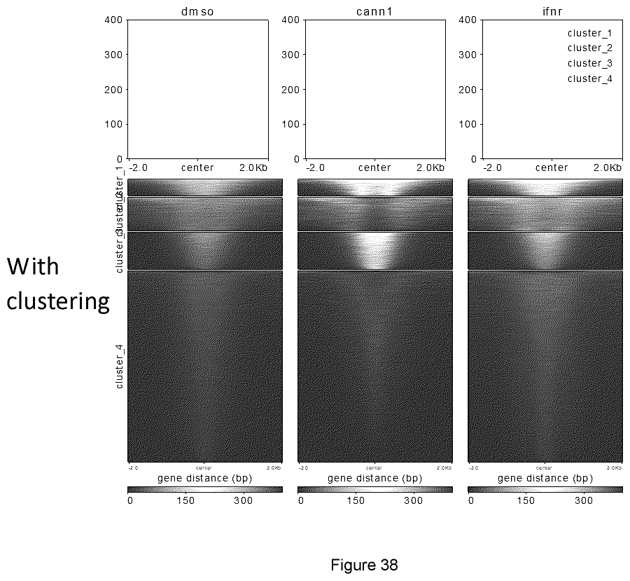

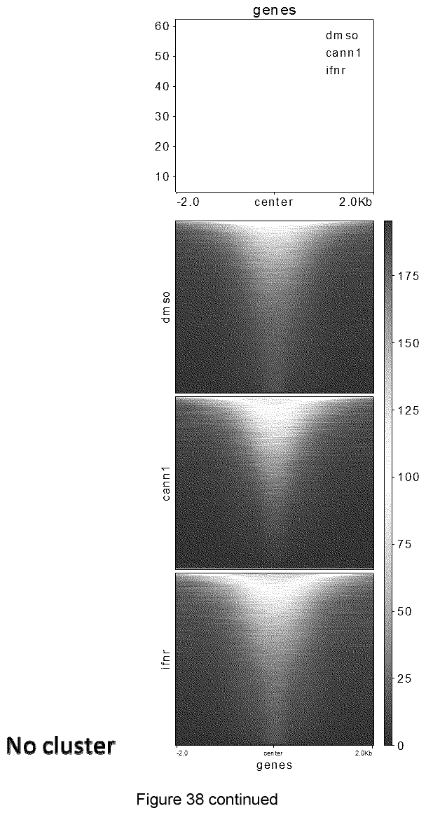

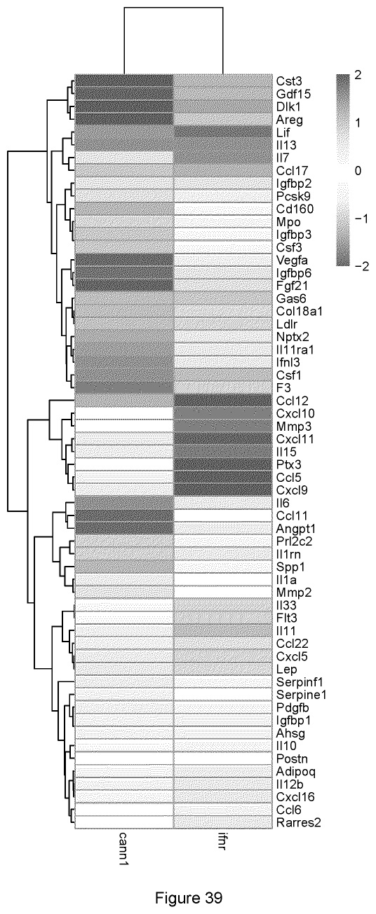

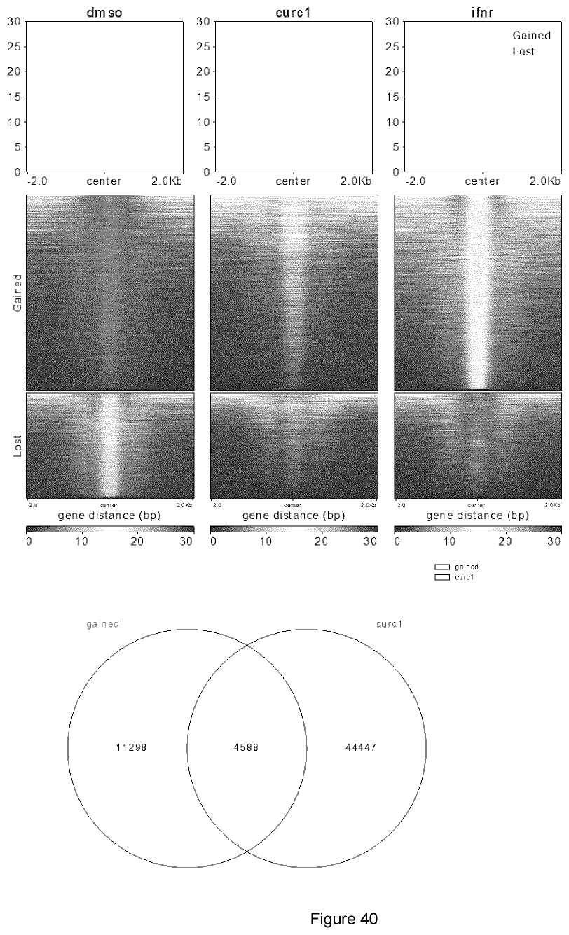

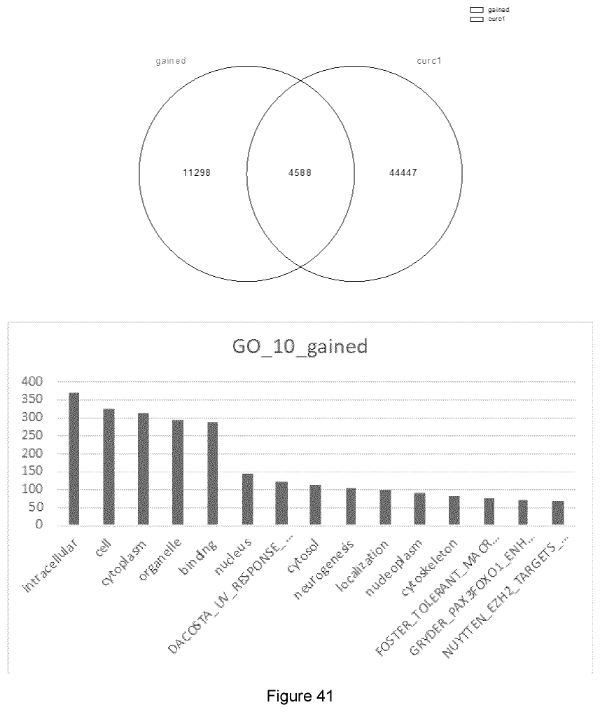

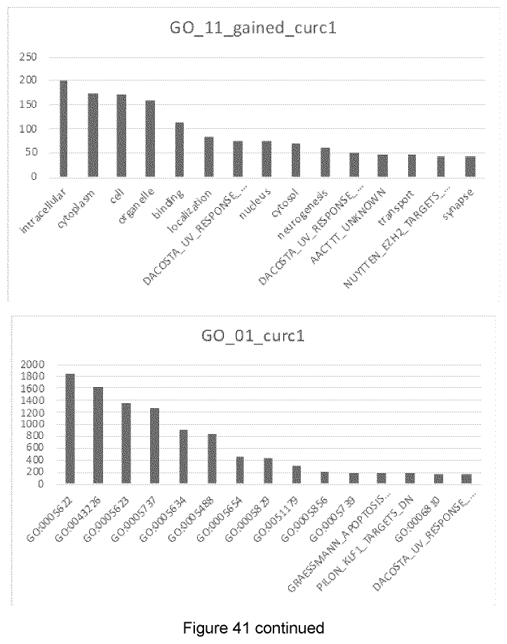

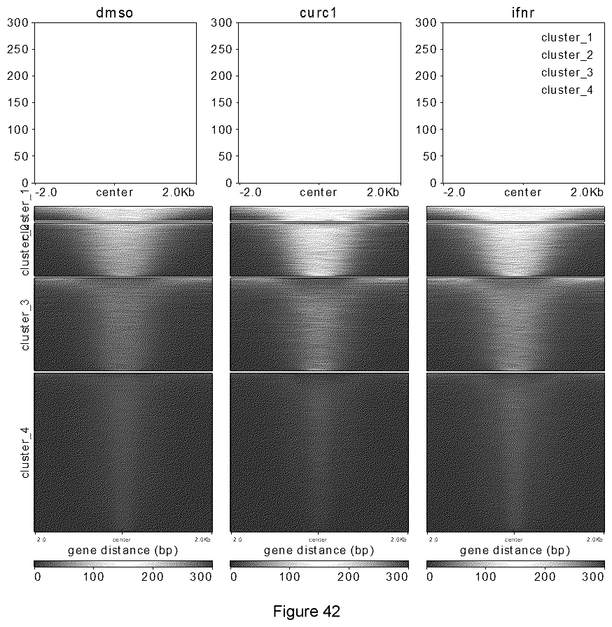

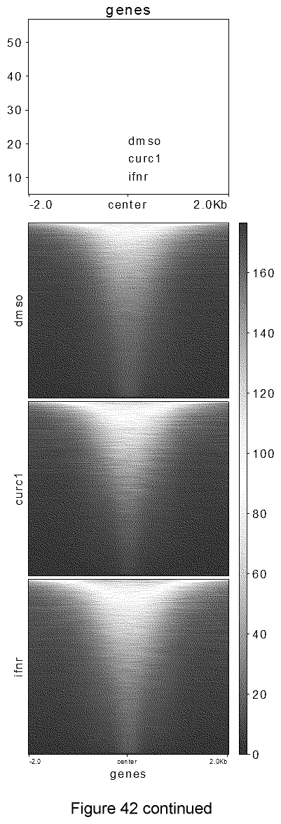

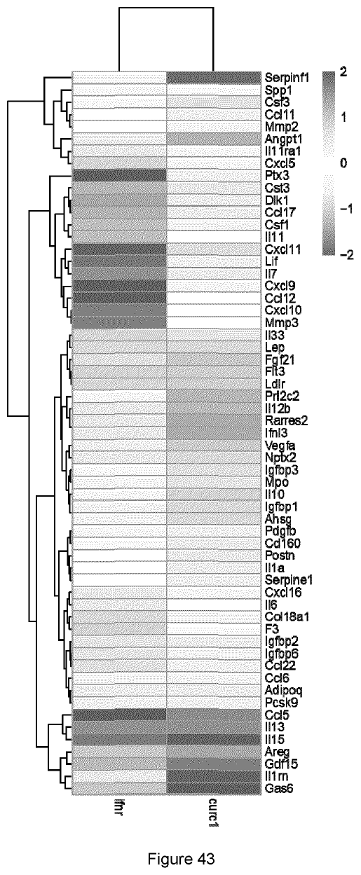

Effects of P02-113 and P03-97-1 on class I/II histone deacetylase activity: Due to the structural similarity of the curcuphenol analogues, P02-113 and P03-97-1, to a previously described HDACi, TSA, it was hypothesized that these molecules could be acting through a similar mechanism. To test this hypothesis, we evaluated the ability of P02-113 and P03-97-1 to affect the class I/II HDAC activity. Interestingly the compounds, P02-113 and P03-97-1 exhibited the opposite effect to what was hypothesized and showed an increase in class I/II HDAC activity (FIG. 29A). Even at the lowest concentrations of 1 nM to 100 nM, there was an induction of HDAC activity. Both compounds showed a peak in HDAC activity around 180 nM, while P02-113 started to reduce the effect at higher concentrations. P03-97-1 maintained peak levels of HDAC activity until the highest concentration of 1 uM suggesting a stronger effect. The stronger effect exhibited by P03-97-1 could be due to several factors including stronger binding affinities to HDAC enzymes, or better ability to enter A9 cells, however the exact reason remains to be determined.

Next, the activity of individual purified recombinant HDACs was evaluated. No significant change in the activities of the class I HDACs 1,2 and 3 were observed at the concentrations tested for both P02-113 and P03-97-1. For compound P02-113, the class I HDAC8 showed more variable results with no change in HDAC8 activity at higher concentrations but at concentrations of 0.3 uM and below inhibition was seen, that was similar to the HDACi exhibited by TSA (FIG. 29B). P03-97-1 also followed a similar pattern with no change in activity at higher concentrations but at the lowest concentration 0.02 uM an inhibitory phenotype was seen. This indicates that the analogues P02-113 and P03-97-1 act as inhibitors to HDAC8 but not for other class I enzymes. Another interesting factor that correlates with the inhibitory effects of P02-113 and PO-3-97-1, is that HDACs 1-3 are limited to the nucleus whereas HDAC8 is the only class I also found in the cytosol.

The class II HDAC family encompasses HDACs 4 through 10 excluding HDAC8. P02-113 and PO-3-97-1 did not affect the activity of the class II HDACs 4, 6, 7 and 9. On the other hand, the activities of both HDAC 5 and 10 were enhanced upon treatment with the curcuphenol analogues (FIG. 6C). Additionally, no effect was observed in the activities of the class III enzyme SIRT1 nor on the class IV enzyme HDAC11.

### DISCUSSION

A novel cell-based high-throughput screening assay designed to identify compounds that induce the expression of the APM components, TAP-1 and surface MHC I molecules, in metastatic prostate and lung carcinomas has been developed. The assay has been used to screen a marine invertebrate natural product extract library resulting in a number of promising hits. Assay guided fractionation of an extract of the sponge Halichindria sp. collected in the Philippines showed that the natural product curcuphenol significantly increased surface MHC I molecules in cancer cell lines in vitro. We have shown that curcuphenol selectively inhibits HDAC 8 and activates the HDACs 5 and 10 and this may be related to its induction of TAP-1 and surface MHC-I molecules in cancer cells.

Natural product libraries offer an excellent source of new compounds that have potential for HDAC modifying activities. Extracts may be isolated from common daily entities such as spices and herbs or they may come from more distant resources, like the depths of the oceans. While spices are typically thought of as staples in cooking, there have been numerous spices identified that have either anti-cancerous properties or can reduce tumour growth that include: cumin, saffron, turmeric, green and black tea and flaxseed that contain curcumin⁴⁵⁻⁴⁸. Another common source of natural therapeutics is herbs, which are a rich source of secondary metabolites including: polyphenols, flavonoids and brassinosteriods⁴⁹. However, of all the natural resources the marine environment dominates in diversity of both biologics and chemicals^{50, 51}. Therefore, screening of extracts from our natural resources remains the greatest source of novel therapeutics that may reduce cancer growth and metastasis.

The work of Stutman in 1974 had momentarily extinguished the concept of T cells mediating immune surveillance⁹ by showing that there was no difference between the growth of tumours in athymic nude mice lacking T cells versus wild-type animals¹⁰. However, Stutman was unaware that these studies were conducted with tumours that lacked APM function and were therefore invisible to host T cell recognition. In 2001, a study done by R. D Schreiber's group¹¹ showed that immune-incompetent Rag2 knockout (Rag2−/−)mice, which do not develop T cells, B cells and NK T cells, or Stat−/− mice, which lack the IFN gamma receptor gene, developed more chemically induced sarcomas much more rapidly than wild type mice^{9, 11}. This was widely hailed as substantial evidence supporting immune surveillance⁹. However, since the animals that Schreiber's group studied lacked T and B cells and NK T cells, in the context of Stutman's study, the deduced conclusion would be that B cells and NK T cells mediate immune surveillance. Fortunately, in the year previous, Alimonte et al,¹² directly repudiated Stutman's study by examining the growth APM competent, chemically induced tumours in athymic nude mice lacking T cells versus wild-type animals. Thus Alimonte et al,¹² demonstrated conclusively for the first time that T cells are required for immune surveillance, as is the coincident expression of functional APM and MHC-I in a tumour: the rules of engagement.

The selective pressure of immune surveillance on genetically unstable tumour populations may yield tumours that have lost expression of APM components, often resulting in reduced assembly of functional major histocompatibility complex (MHC or HLA) molecules (Alimonti et al.⁴). Several types of cancer, including breast cancer^{12, 13} renal carcinoma¹⁴, melanoma¹⁵, ¹⁶, colorectal carcinoma¹⁷, head and neck squamous cell cancer⁸, cervical cancer¹⁹ and finally prostate carcinoma exhibit APM deficits and show a clear correlation between human leukocyte antigen (HLA) down-regulation and poor prognosis²⁰⁻²².

Depending on the tumour type, the loss of APM components and functional MHC-I (HLA-1) molecules with an immune escape may be present in up to 90% of patients and is associated with tumour aggressiveness and increased metastatic potential²⁰⁻²². Furthermore, tumours may become ‘invisible’ or unrecognizable by CTLs and may also become refractory to emerging immunotherapeutics such as CAR-T cells and immune checkpoint blockage inhibitors. Currently only 15-30% of patients do respond to current immunotherapies⁵³. Discovering new therapeutics candidates like those described here which overcome immune escape and can augment the emerging immunotherapy modalities is a priority. Therefore, combination therapies may be key in the future, where the addition of drugs targeting the up regulation of the APM will be utilized.

Our results indicate that multiple extracts isolated from sea sponges are able to induce a significant increase in surface MHC-I expression, while at the same time exhibiting low cytotoxicity. The chemical structure of the active component in one of the sponge extracts has been identified here as curcuphenol, which has also been isolated from turmeric, a commonly used cooking spice. Curcuphenol can be found as one of two enantiomers: S-(+) and R-(−) curcuphenol⁵⁵⁻⁶². Curcuphenol pharmacophore analogs were synthesized in an attempt to find more efficacious analogs. Initial studies indicate that the two curcuphenol-based compounds, P02-113 and P03-97-1, were well tolerated in vivo and there was no toxicity in animals at the doses that were studied. Treatment of metastatic tumour-bearing mice with the compounds resulted in significant reduction in the mean tumour volume. The compound, P03-97-1 also induced a significant infiltration of CTLs into the tumour, indicating the tumour was being recognized by the adaptive immune system. Overall, P03-97-1 exhibited a stronger in vivo effect, which may be attributed to a better ability to enter A9 cells, however the exact reason remains to be determined. Due to the stronger anti-cancer properties of P03-97-1, as well as increased stimulation of CTLs into tumours, it may be a strong candidate for future combination therapies where it could induce the expression of the MHC-I molecules and increase the survival rate for patients whose cancers show an immune-evasive phenotype due to reduced levels of the APM. However, optimization of the dosing of P03-97-1 and further chemical modification of the scaffold to increase its plasma half-life will be required as it was found to have a high rate of elimination from mouse plasma and becomes undetectable after six hours.

The antigen processing genes in many metastatic cancers are under epigenetic control, this indicated that the most fertile avenue of further exploration would be to assess if curcuphenols have a hitherto, undescribed epigenetic modifying activity. To explore this possibility, we established HDAC assays and tested effect of Curcuphenols and controls on these HDAC assays. Due to the very similar structure of the curcuphenol analogues to a known HDACi, TSA, which promotes the expression of MHC-I in the A9 cell line²⁹ it was predicted that the analogues were acting through a similar mechanism. However, upon a generalized class I/II HDAC luminescence assay to measure HDAC activity, using A9 cells, the opposite effect was discovered and HDAC activity was enhanced. This HDAC enhancement (HDACe) is a novel trait that has never been seen in the literature for class I/II HDACs, however, there is one known HDAC activator for the class III HDACs, Resveratrol, which indirectly acts upon SIRT1⁷³. To determine whether P02-113 and P03-97-1 were in fact directly interacting with HDAC enzymes to promote activity, individual purified recombinant HDACs were assessed following treatment with the analogues. While the majority of HDAC enzymes did not show a change in activity, one enzyme, HDAC8, showed inhibition. This is interesting as this is the only class I HDAC that is known to exist in both the nucleus and cytoplasm and diverged early in evolution from the other class I enzymes⁷⁴. This very specific targeted inhibition of HDAC8 is a unique feature of the compounds as the majority of HDACi being developed show pan HDACi. However, these analogues present a more targeted and optimal affinity than has been seen before. Interestingly, it is known that increased HDAC8 activity is associated with cancer as well as in other diseases including neurodegenerative disorders, metabolic deregulation, autoimmune and inflammatory diseases⁷⁴. Therefore, these compounds hold potential as specific HDAC8 inhibitors. In regard to the APM, it has been demonstrated that HDAC8 acts as a scaffold for cAMP responsive element binding protein (CREB), a known transcriptional up-regulator of TAP-1 and MHC-1, where upon over-expression of HDAC8, CREB phosphorylation became decreased along with its transcriptional activity⁷⁵. To determine if the increased expression of the APM is directly correlated with the inhibitory activity of P02-113 and P03-97-1 on HDAC8, further experiments in which HDAC8 is knocked down in the TC-1 cell line and APM expression is measured will be required. HDAC8 has previously been knocked-down using RNA interference in lung, colon and cervical cancer cell lines resulting in reduced proliferation while its over-expression promotes proliferation and inhibits apoptosis in hepatocellular carcinoma, however the APM remains to be examined^{76, 77}.

In contrast to an inhibited HDAC activity, there were two HDACs (5 and 10), which showed an enhanced activity upon treatment with P02-113 and P03-97-1. These are most likely the HDAC candidates showing an increase in activity in the generalized HDAC class I/II assay preformed on the A9 cell line. This is a unique finding as HDACs are currently viewed as being overactive in cancer to decrease the expression of cancer preventing genes. However, reductions in activity of both HDACs 5 and 10 have been implemented in advanced stages of lung cancer and are correlated with poor outcome^{78, 79}. Interestingly, previous studies that have down-regulated HDAC5 using siRNA found that there was a pro-angiogenic effect due to increased endothelial cell migration, sprouting, and tube formation⁸⁰. As for HDAC10, there has been significantly more research done in relation to its activity in cancer. Decreases in HDAC10 activity has been correlated with more aggressive malignancies in B cell and gastric cancers and has been correlated with metastasis in gastric cancer and squamous cell carcinomas⁸¹⁻⁸³. A mechanism has also been demonstrated for HDAC10 involvement with metastasis, as it is known to suppress matrix metalloproteases 2 and 9 that are critical for cancer cell invasion and metastasis⁸¹. Therefore, future work to establish if HDAC5 and HDAC10 are crucial to the regulation of the APM will be fundamental to understand if P02-113 and P03-97-1 exhibit up-regulation through the enhancement of HDACs 5 and 10.

In summary, we have developed a novel high-throughput cell-based assay to screen and identify compounds in a library made from marine invertebrate extracts that induce the expression of the APM components, TAP-1 and MHC-I molecules, in metastatic prostate and lung carcinomas. Curcuphenol a component of turmeric used in curry spices has been identified as the active component in the most promising extract and curcuphenol analogs have been prepared that have increased ease of synthesis and enhanced biological performance. These curcuphenol-based compounds possess novel HDAC enhancing (HDACe) activity, and reverse immune escape in metastatic tumours by enhancing the expression of APM components. They are well tolerated in vivo, and treatment of metastatic tumour-bearing mice with these compounds resulted in significant reduction in the mean tumour volume. These studies explain and highlight the potential medicinal value of common components spices used in the preparation of foods.

Materials and Methods

Marine extract library. The marine invertebrate extract collection was prepared from more than 5,000 frozen sponge, tunicate, and mollusc specimens collected by SCUBA diving at 0-40 meter depths at locations in regions of high marine biodiversity in Papua New Guinea, Indonesia, Thailand, Sri Lanka, Dominica, Brazil, Canada (British Columbia), South Africa, the Philippines, and Norway that were tagged with a global positioning system (GPS)⁷⁴. Specimens were frozen immediately after collection in the field and transported frozen to Vancouver. One hundred grams of each frozen invertebrate was thawed and extracted directly with methanol or lyophilized followed by extraction with methanol. Approximately 2 mg of each concentrated crude methanol extract was dissolved in DMSO and stored in 96-well plates at −20° C. A selection of these plates, containing more than 400 crude sponge extracts, was used in the in vitro screening assays.

Cell lines. PA and LMD murine prostate carcinoma cell lines. PA and LMD cell lines are models of non-metastatic and metastatic prostate cancer, respectively. PA is a primary murine prostate cancer cell line derived from a 129/Sv mouse using a mouse prostate reconstitution model system that displays high expression of MHC-I. LMD is a metastatic derivative of PA which is deficient in the expression of TAP-1 and MHC-I⁷⁵. These cell lines were provided by Dr. T. C. Thompson, Baylor College of Medicine, Houston) and cultured as previously described 75.

TC-1 and A9 murine lung tumour model. The TC-1 cell line is a murine lung tumour model derived from primary lung epithelial cells of C57BL/6 mice immortalized using the amphotropic retrovirus vector LXSN16 carrying Human Papillomavirus E6/E7, and subsequently transformed with pVEJB plasmid expressing the activated human c-Ha-ras oncogene. TC-1 cells display high expression of TAP-1 and MHC-I. The cell line A9 was derived from the TC-1 tumour cell line and display spontaneous down-regulation of MHC-I (H2-K1) by immunoselection in vivo after immunization of animals bearing the original TC-1 parental cells with modified HPV16 E7 genes against mouse oncogenic TC-1 cell resulting in the sub-lines with down regulated expression of MHC-I molecules⁷⁶. A9 cells have been shown to be metastatic in a mouse model⁷⁷. The cells were cultured as previously described⁷⁶.

LMD reporter cell line. For the initial investigation of cancer cells, the LMD TAP-deficient metastatic prostate carcinoma cell line was transfected with a vector expressing enhanced green fluorescent protein (EGFP) under the TAP-1 promoter 44 to generate the LMD:TAP-1 cells. LMD:TAP-1 cells were maintained in DMEM supplemented with 10% fetal bovine serum, and 1 mg/mL of G418. LMD:TAP-1 were stimulated with 100 ng/mL IFN-γ and sorted based on high EGFP expression. Single cells were sorted into 96-well plates and incubated for 12 to 14 days until colonies could be observed. The clonal population that showed low GFP intensity in non-stimulated conditions and highest GFP intensity upon IFN-γ stimulation was further used in the assays.

Cell-based screening assay. LMD:TAP-1 cells were seeded in PerkinElmer View 96-well plates at 3500 cells per well. Twenty four hours after seeding, cells were cultured in the presence of the indicated concentrations of the marine extracts, 10 ng/mL of IFN-γ or 1% DMSO control. Plates were incubated for 48 hours at 37° C. in a 5% CO2 incubator. The medium was removed and cells fixed with 4% (v/v) paraformaldehyde containing 500 ng/mL Hoechst 33342 (Molecular Probes). Fixed cells were stored in PBS at 4° C. until further analysis. Image acquisition, segmentation and analysis of micro plates were carried out using the Cellomics™ Arrayscan V^{TI} automated fluorescence imager (Thermo Fisher Scientific). Images from 12 fields were acquired using a 20× objective in the Hoechst and GFP (XF-100 filter) channels (auto-focus, fixed exposure time). The target activation algorithm was used to identify the nuclei based on Hoechst fluorescence intensity, apply a cytoplasmic mask and quantitate GFP fluorescence intensity within the cytoplasmic mask area. Average GFP fluorescence intensity (intensity per cell per pixel) and total number of cells per well were determined. To assess the quality of the screening assay, the Z′-factor⁷⁸ was calculated as 1-(3×δp+3×δn)/(|μp−μn|), where μp, δp, μn and δn are the means (μ) and standard deviations of both the positive (p) and negative (n) controls (10 ng/mL IFN-γ and 1% DMSO, respectively).

Evaluation of MHC-I surface expression by flow cytometry. LMD:TAP-1 cells or A9 cells were plated in 6 well plates at a concentration of 10,000 cells per well in a 2 mL volume. The next day, cells were treated with the indicated concentrations of the compounds and incubated for 48 hours at 37° C. After incubation, the cells were trypsinized, washed and stained with APC-conjugated anti-mouse MHC-I (specifically anti-H-2K^{b}) antibody (Biolegend) and assessed by flow cytometry analysis. As a positive control, LMD or A9 cells were treated with either IFNγ (50 ng/mL) or TSA (100 ng/mL) for 48 hours, to induce surface MHC-I expression, and vehicle alone (1% DMSO) was used as a negative control.

Analysis of chemical structure and isolation of active ingredient(s). Extracts showing potential activity were subjected to bioassay-guided fractionation to give pure active natural products for further biological examination. Fractionation was performed in multiple rounds to ultimately identify a single active compound. Each sub-fraction was then tested using the cell-based screening assay to identify the active sub-fraction and then further analyzed by flow cytometry to verify MHC-I surface expression.

Isolation of curcuphenol from sponge sample 76018—extract #2. Specimens of the massive orange sponge, Halichondria sp., were collected by hand using SCUBA at Solong-on, Siquijor Island, Philippines¹⁰. A voucher sample has been deposited at the Netherlands Centre for Biodiversity Naturalis in Leiden, the Netherlands (voucher number: RMNH POR. 5872). Lyophilized sponge material (15 g) was extracted with methanol (3×50 mL) at room temperature. Bioassay-guided fractionation of the crude methanol extract as described in the Supplementary Information identified curcuphenol as the active component. Analysis of the 1D and 2D nuclear magnetic resonance (NMR) and mass spectrometry (MS) data collected for the curcuphenol sample obtained from the Halichondria sp. unambiguously identified its constitution, but its absolute configuration was not determined.

Synthesis of racemic curcuphenol and pharmacophore analogs. Racemic curcuphenol was synthesized to provide sufficient material for biological evaluation and the curcuphenol structural analogs P02-113 and P03-97-1 were synthesized in an attempt to increase the bioactivity of the curcuphenol lead structure. The synthetic details can be found in the Supplementary Information.

In vivo efficacy studies. Maximum tolerated dose (MTD). The maximum tolerated dose for the selected compounds, P02-113 and P03-97-1, were assessed in vivo. C57BI/6 mice were injected intraperitoneally (i.p.) with the test compounds at 3 different concentrations: 1.0 mg/kg (n=3), 3.5 mg/kg (n=3) and 5.2 mg/kg (n=3). Mice were then assessed for 14 days for clinical signs of toxicity and at the end point a necropsy was performed. The highest MTD dose showed no adverse effects and was used for further testing.

Pharmacokinetic study. The compounds, P02-113 and P03-97-1, were evaluated at three time points following injection i.p. of the either P02-113 or P03-97-1 at a concentration of 5.2 mg/kg. Three mice were used for each time point, with a total of nine mice per compound. Time points were strategically chosen based on the similar structure of the compounds to TSA, an HDACi, which is known to augment TAP and MHC-I expression in metastatic tumours and to be metabolized at a high rate. The chosen time points were 5 minutes, 10 minutes, 1 hour and 6 hours.

Treatment of tumour-bearing mice with identified compounds. 5×10⁴ A9 cells suspended in HBSS were subcutaneously (s.c.) transplanted into the right flank of 32 eight-week-old female C57BI/6 mice, as previously described 75. Starting at day 7 after tumour injection, mice from each tumour group were treated daily by i.p. injection with either the one of the identified compounds (n=8 for each compound), TSA positive control (n=8), or with vehicle alone (n=8) for two weeks. Body weight and tumours (once established) were measured every 2-4 days (more often as tumour size increased). Tumours were measured using calipers and volume was calculated as following: tumour volume=length×width². The tumour growth rate was assessed using methods previously described²⁸.

Survival curves. Survival for mice receiving A9 tumours was based on an assessment of overall weight of the mouse and tumour volume, where mice were euthanized if they lost 20% of their starting weight or tumours grew beyond 1 cm³ in size, in order to comply with animal ethics guidelines.

Analysis of tumour-infiltrating lymphocytes (TILs). Tumour-infiltrating T lymphocyte (either CD4⁺ or CD8⁺ T cells) infiltration was evaluated in tumours of mice following 2-week treatment with either compounds or controls. Tumours at the site of initial injection were removed from tumour-bearing mice. Following tumour dissociation in the presence of collagenase A (Roche) and erythrocyte lysis, the tumour cells were then washed and prepared as single-cell suspensions to detect TILs. Before staining with antibodies, the cells were incubated with Fc Blocker (Ebiosciences) for 20 minutes at 4° C. The tumour cells were then washed and stained with anti-CD4-APC (Biolegend), anti-CD8-PECy7 (eBiosciences) and 7-AAD (Biolegend) viability stain. Using flow cytometry, 7-AAD positive dead cells were gated out and the remaining population was assessed for CD4⁺ and CD8⁺ expression. Data were acquired using a BD™ LSR II flow cytometer (BD Biosciences) with FACSDiva™ software and analyzed with FlowJo software (Treestar).

HDAC assays. To assess the effect of the compounds on the relative activity of histone deacetylase (HDAC) class I and II enzymes in the A9 cell line, we used the HDAC-Glo™ I/II Assay and Screening System (Promega). The linear range was established for the A9 cells following the manufacturer's instructions. Thirty thousand cells per well were plated in clear-bottom 96-well plates (Perkin Elmer) and plates were incubated at 37° C. After 24 hours, cells were treated with 25 nM of TSA (positive control), 1% DMSO (negative control), or a range of dilutions of P02-113 or P03-97-1 (5 to 0.02 μM) and incubated for 30 minutes. Cell culture media was used as a blank control and HeLa cells provided in the HDAC assay kit were used as a positive control. HDAC class I/II reagent was then added and incubated for 30 minutes before luminescence was measured using the Infinite M200 (Tecan) and i-control software (Tecan).

To evaluate the effect of the compounds on specific HDACs, their activity was assessed with purified HDAC enzymes from all classes I, II, and IV, as well as a select member of HDAC class III (SIRT1). HDACs 1-9 and SIRT1 were evaluated using HDAC Fluorogenic Assay Kits (BPS Biosciences) following the manufacturer's recommendations. Compound treatment started at 5 μM and was two-fold diluted to a concentration 0.02 μM. Alternatively, HDAC 10 and 11 assays (BPS Biosciences) were optimized to be used with the HDAC-Glo™ I/II Assay and Screening System (Promega). The assays were measured using the Synergy HI hybrid reader (BioTek) and Gen5 software (Bio-Tek). For all assays, vehicle (1% DMSO) was used as a negative control and TSA (25 nM) was used as a positive control, except the SIRT1 assay where nicotinamide (5 mM) was used as positive control. To calculate the fold change in HDAC activity, the values from each treated well were divided by the relative mean of activity of the specific HDAC being measured.

Extraction of the Sponge, Halichondria Sp. and Isolation of Curcuphenol.

Freshly collected sponge specimens were frozen on site and transported frozen. Lyophilized sponge material (15 g) was cut into small pieces, immersed in and subsequently extracted repeatedly with MeOH (3×50 mL) at room temperature. The combined methanolic extracts were concentrated in vacuo, and the resultant extract was then partitioned between EtOAc (3×5 mL) and H₂O (15 mL). The combined EtOAc extract was evaporated to dryness, and the resulting active oil was chromatographed on Sephadex LH-20 with 4:1 MeOH/CH₂Cl₂ as eluent to give 6 mg of curcuphenol as a clear oil. Analysis of the 1D and 2D nuclear magnetic resonance (NMR) and mass spectrometry (MS) data collected for the curcuphenol sample unambiguously identified its constitution, but its absolute configuration was not determined.

Curcuphenol. Isolated as a clear oil; ¹H (600 MHz, DMSO-d₆) δ1.08 (d, J=6.8 Hz, 3H), 1.44 (m, 1H), 1.47 (s, 3H), 1.56 (m, 1H), 1.61 (s, 3H), 1.82 (m, 2H), 2.15 (s, 3H), 2.99 (m, 1H), 5.07 (bt, J=7.1 Hz, 1H), 6.53 (d, J=7.6 Hz, 1H), 6.56 (s, 1H), 6.91 (d, J=7.6 Hz, 1H), 9.00 (s, 1H) ppm; ¹³C (150 MHz, DMSO-d₆) δ 17.5, 20.7, 21.0, 25.5, 25.8, 30.9, 36.6, 115.6, 119.6, 124.6, 126.4, 129.9, 130.4, 135.2, 154.4 ppm; positive ion LRESIMS [M+Na]⁺ m/z 241.2 (calcd for C₁₅H₂₂ONa, 241.1568).

Experimental Procedure for the Synthesis of Curcuphenol Analogs.

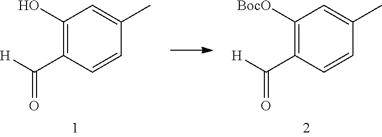

To a solution of 2-hydroxy-4-methylbenzaldehyde (solution 1) (231.0 mg, 1.65 mmol) in CH₂Cl₂ (2 mL) were added a solution of Boc₂O (381.0 mg, 1.73 mmol) in CH₂Cl₂ (1 mL), DMAP (20.3 mg, 0.165 mmol) and i-Pr₂NEt (0.21 mL, 1.19 mmol) at room temperature. After stirring for 3.5 h, the reaction was quenched with saturated aqeuous NH₄Cl solution. The mixture was extracted with CH₂Cl₂ for three times. The combined organic extracts were washed with brine, dried over MgSO₄ and evaporated under vacuum. The residue was purified by flash chromatography (silica gel, step gradient from 0:100 EtOAc/hexanes to 5:100 EtOAc/hexanes) to give compound 2 (372.0 mg, 96%) as a colorless oil.

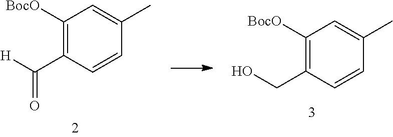

To a solution of 2 (372.0 mg, 1.58 mmol) in THF (15 mL) was added BH₃.Me₂S (0.17 mL, 1.72 mmol) at 0° C. The cooling bath was left in place but not recharged, and the mixture was stirred for 3 h. The reaction was quenched with 0.1 M HCl and extracted with EtOAc. The combined organic extracts were washed with brine, dried over MgSO₄ and evaporated under vacuum. The residue was purified by flash chromatography (silica gel, 25:100 EtOAc/hexanes) to give compound 3 (360.7 mg, 96%) as a colorless oil. ¹H NMR (400 MHz, CDCl₃) δ 7.32 (d, J=7.6 Hz, 1H), 7.05 (d, J=7.6 Hz, 1H), 6.94 (s, 1H), 4.55 (d, J=6.0 Hz, 1H), 2.34 (s, 4H), 1.55 (s, 9H). ¹³C NMR (100 MHz, CDCl₃) δ 152.7, 148.8, 139.4, 130.0, 129.6, 127.4, 122.6, 83.9, 60.3, 27.8, 21.2.

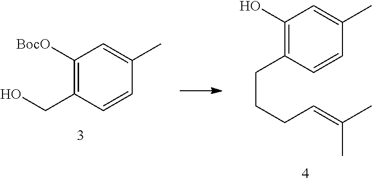

To a mixture of Mg turnings (94.0 mg, 3.92 mmol) and I₂ (tiny) in Et₂O (0.5 mL) was added several drops of a solution of 5-bromo-2-methyl-2-pentene (0.43 mL, 3.21 mmol) in Et₂O (2.5 mL). After stirring for a few minutes, the yellow solution was turned into colorless solution, then the bromide solution was added dropwise over 50 min. The reaction mixture was then stirred under reflux for 1 h. To solution of 3 (110.2 mg, 0.46 mmol) in Et₂O (4 mL) was added the freshly prepared Grignard reagent at −78° C. The reaction was allowed to warm to room temperature over 3 h before quenching with saturated aqeuous NH₄Cl solution. The mixture was extracted with Et₂O. The combined organic extracts were washed with brine, dried over MgSO₄ and evaporated under vacuum. The residue was purified by flash chromatography (silica gel, step gradient from 2:100 Et₂O/hexanes to 4:100 Et₂O/hexanes) to give compound 4 (PC-02-113) (55.4 mg, 59%) as a colorless oil. ¹H NMR (400 MHz, CDCl₃) δ 7.00 (d, J=7.6 Hz, 1H), 6.69 (d, J=7.6 Hz, 1H), 6.60 (s, 1H), 5.18 (t, J=7.2 Hz, 1H), 4.69 (s, 1H), 2.57 (t, J=7.6 Hz, 2H), 2.28 (s, 3H), 2.06 (q, J=6.8 Hz, 2H), 1.72 (s, 3H), 1.62-1.72 (m, 2H), 1.62 (s, 3H). ¹³C NMR (100 MHz, CDCl₃) δ 153.5, 137.2, 132.4, 130.2, 125.4, 124.6, 121.7, 116.2, 30.2, 29.2, 27.9, 26.0, 21.1, 18.0.

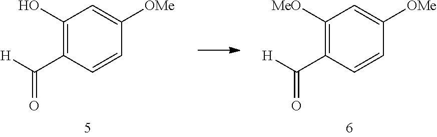 [SMILES: COc1ccc(C=O)c(O)c1 ; COc1ccc(C=O)c(OC)c1]

To a suspension of NaH (172.6 mg, 60% in mineral oil, 4.32 mmol) in DMF/THF (5.4 mL, 4:1 v/v) was slowly added a solution of 2-hydroxy-4-methoxybenzaldehyde (solution 5) (546.6 mg, 3.60 mmol) and MeI (0.46 mL, 7.32 mmol) in THF (3.6 mL) at 0° C. The cooling bath was left in place but not recharged, and the mixture was stirred for 18 h. The mixture was then diluted with Et₂O and washed with H₂O. The organic extract was dried over MgSO₄ and evaporated under vacuum. The residue was purified by flash chromatography (silica gel, step gradient from 5:100 EtOAc/hexanes to 15:100 EtOAc/hexanes) to give compound 6 (552.5 mg, 93%) as a white solid. ¹H NMR (400 MHz, CDCl₃) δ 10.28 (s, 1H), 7.79 (d, J=8.8 Hz, 1H), 6.53 (dd, J=1.2, 8.4 Hz, 1H), 6.43 (d, J=2.0 Hz, 1H), 3.89 (s, 3H), 3.86 (s, 3H). ¹³C NMR (100 MHz, CDCl₃) δ 188.5, 166.4, 163.8, 130.9, 119.2, 106.0, 98.1, 55.81, 55.79.

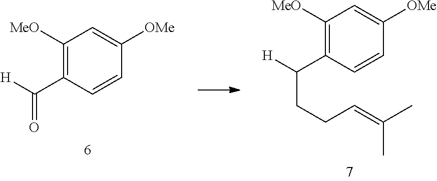 [SMILES: COc1ccc(C=O)c(OC)c1 ; COc1ccc(CCCC=C(C)C)c(OC)c1]

To a mixture of Mg turnings (189.4 mg, 7.89 mmol) and I₂ (tiny) in Et₂O (1.0 mL) was added several drops of a solution of 5-bromo-2-methyl-2-pentene (0.88 mL, 6.57 mmol) in Et₂O (4.8 mL). After stirring for a few minutes, the yellow solution was turned into colorless solution, then the bromide solution was added dropwise over 1 h. The reaction mixture was then stirred under reflux for 1 h. To solution of 6 (272.3 mg, 1.64 mmol) in THF (8 mL) was added the freshly prepared Grignard reagent at 0° C. The cooling bath was left in place but not recharged, and the mixture was stirred for 18 h. The reaction was quenched with saturated aqeuous NH₄Cl solution and extracted with EtOAc for three times. The combined organic extracts were washed with brine, dried over MgSO₄ and evaporated under vacuum. The residue was purified by flash chromatography (silica gel, step gradient from 5:100 EtOAc/hexanes to 15:100 EtOAc/hexanes) to give compound 7 (389.6 mg, 95%) as a colorless oil. ¹H NMR (400 MHz, CDCl₃) δ 7.19 (d, J=8.4 Hz, 1H), 6.44-6.48 (m, 2H), 5.15 (tt, J=1.2, 7.2 Hz, 1H), 4.80 (t, J=6.4 Hz, 1H), 3.81 (s, 3H), 3.79 (s, 3H), 2.53 (bs, 1H), 1.98-2.16 (m, 2H), 1.72-1.88 (m, 2H), 1.69 (s, 3H), 1.59 (s, 3H). ¹³C NMR (100 MHz, CDCl₃) δ 160.2, 157.9, 132.0, 127.8, 125.3, 124.4, 104.2, 98.8, 70.5, 55.5, 55.4, 37.4, 25.9, 25.0, 17.9. HRESIMS [M+Na]⁺ m/z 273.1462 (calcd for C₁₅H₂₂O₃Na, 273.1467).

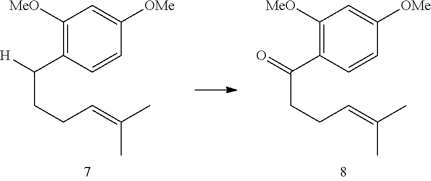 [SMILES: COc1ccc(CCCC=C(C)C)c(OC)c1 ; COc1ccc(C(=O)CCC=C(C)C)c(OC)c1]

To a solution of 7 (327.1 mg, 1.31 mmol) in CH₂Cl₂ (10 mL) was added DMP (714.9 mg, 1.64 mmol) at room temperature. The mixture was stirred for 30 min, and TLC analysis showed a complete disappearance of the starting material. Then saturated aqueous NaHCO₃ solution was added and the mixture was extracted with CH₂Cl₂ for three times. The combined organic extracts were washed with brine, dried over MgSO₄ and evaporated under vacuum. The residue was purified by flash chromatography (silica gel, step gradient from 0:100 EtOAc/hexanes to 8:100 EtOAc/hexanes) to give compound 8 (280.3 mg, 86%) as a colorless oil. ¹H NMR (400 MHz, CDCl₃) δ 7.79 (d, J=8.8 Hz, 1H), 6.52 (dd, J=2.0, 8.8 Hz, 1H), 6.45 (d, J=2.0 Hz, 1H), 5.15 (dt, J=1.6, 7.2 Hz, 1H), 3.88 (s, 3H), 3.85 (s, 3H), 2.95 (t, J=7.6 Hz, 2H), 2.34 (q, J=7.6 Hz, 2H), 1.68 (s, 3H), 1.61 (s, 3H). ¹³C NMR (100 MHz, CDCl₃) δ 200.5, 164.4, 160.9, 132.9, 132.2, 123.9, 121.5, 105.2, 98.5, 55.70, 55.65, 43.9, 25.9, 23.5, 17.8. HRESIMS [M+Na]⁺ m/z 271.1309 (calcd for C₁₅H₂₀O₃Na, 271.1310).

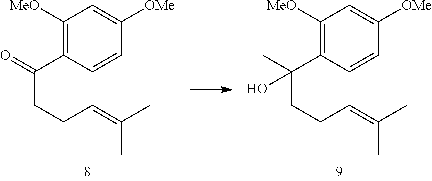 [SMILES: COc1ccc(C(=O)CCC=C(C)C)c(OC)c1 ; COc1ccc(C(C)(O)CCC=C(C)C)c(OC)c1]

To solution of 8 (179.3 mg, 0.72 mmol) in THF (3 mL) was slowly added MeMgBr solution (0.32 mL, 3.0 M in Et₂O, 0.96 mmol) at 0° C. The mixture was stirred at room temperature for 2 h. Then the reaction mixture was cooled to 0° C., and quenched with saturated aqeuous NH₄Cl solution. The mixture was extracted with CH₂Cl₂ for three times. The combined organic extracts were washed with brine, dried over MgSO₄ and evaporated under vacuum.

The residue was purified by flash chromatography (silica gel, step gradient from 0:100 EtOAc/hexanes to 7:100 EtOAc/hexanes) to give compound 9 (128.1 mg, 67%) as a colorless oil. ¹H NMR (400 MHz, CDCl₃) δ 7.21 (d, J=8.4 Hz, 1H), 6.49 (d, J=2.4 Hz, 1H), 6.46 (dd, J=2.4, 8.8 Hz, 1H), 5.08 (t, J=6.8 Hz, 1H), 3.85 (s, 3H), 3.82 (bs, 1H), 3.80 (s, 3H), 1.79-2.01 (m, 4H), 1.65 (s, 3H), 1.54 (s, 3H), 1.51 (s, 3H). ¹³C NMR (100 MHz, CDCl₃) δ 159.8, 157.9, 131.6, 127.6, 127.5, 124.8, 104.1, 99.5, 75.0, 55.5, 42.3, 27.7, 25.8, 23.6, 17.7. HRESIMS [M+Na]⁺ m/z 287.1619 (calcd for C₁₆H₂₄O₃Na, 287.1623).

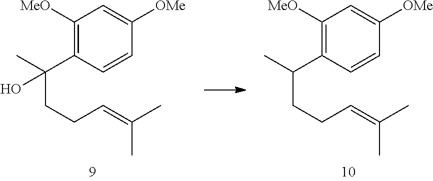 [SMILES: COc1ccc(C(C)(O)CCC=C(C)C)c(OC)c1 ; COc1ccc(C(C)CCC=C(C)C)c(OC)c1]

To solution of 9 (169.3 mg, 0.64 mmol) in CH₂Cl₂ (2 mL) was dropwise added Et₃SiH (0.13 mL, 0.81 mmol) at −78° C. After stirring for 10 min, BF₃OEt₂ (0.12 mL, 0.97 mmol) was added dropwise and stirring was continued for 1 h at −78° C. The mixture was then diluted with CH₂Cl₂ and washed with saturated aqueous NaHCO₃ solution and H₂O until neutral. The organic extract was dried over MgSO₄ and evaporated under vacuum. The residue was purified by flash chromatography (silica gel, step gradient from 0:100 EtOAc/hexanes to 1:100 EtOAc/hexanes) to give compound 10 (135.1 mg, 85%) as a colorless oil. ¹H NMR (400 MHz, CDCl₃) δ 7.06 (d, J=8.0 Hz, 1H), 6.45-6.48 (m, 2H), 5.10-5.15 (m, 1H), 3.802 (s, 3H), 3.797 (s, 3H), 3.10 (sixt, J=7.2 Hz, 1H), 1.83-1.97 (m, 2H), 1.47-1.68 (m, 8H). ¹³C NMR (100 MHz, CDCl₃) δ 158.8, 158.2, 131.3, 128.6, 127.3, 125.1, 104.2, 98.7, 55.54, 55.48, 37.5, 31.5, 26.5, 25.9, 21.4, 17.8. HRESIMS [M+H]⁺ m/z 249.1854 (calcd for C₁₆H₂₅O₂, 249.1855).

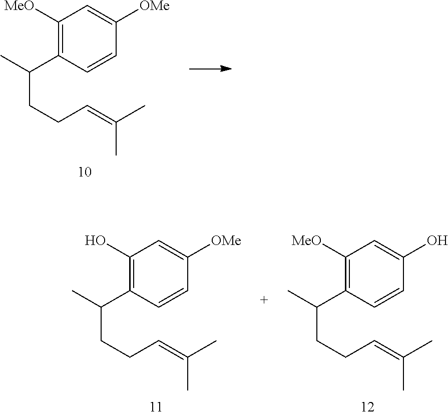 [SMILES: COc1ccc(C(C)CCC=C(C)C)c(OC)c1 ; COc1ccc(C(C)CCC=C(C)C)c(O)c1 ; COc1cc(O)ccc1C(C)CCC=C(C)C]

To NaSEt (489.9 mg, 5.24 mmol) was added DMF (2 mL) at 0° C. The suspension was then warmed up to room temperature and a solution of 10 (110.0 mg, 0.44 mmol) in DMF (1 mL) was added. The mixture was stirred under reflux for 3 h, and then cooled to 0° C. 10% HCl (˜3 mL) and CH₂Cl₂ (˜15 mL) were added at 0° C. The organic layer was washed with H₂O for twice, dried over MgSO₄ and evaporated under vacuum. The residue was purified by flash chromatography (silica gel, step gradient from 0:100 EtOAc/hexanes to 10:100 EtOAc/hexanes) to give compound 11 (PC-03-97-1) (54.2 mg, 52%) as a colorless oil and 12 (PC-03-97-2) (30.4 mg, 29%) as a light yellow oil. For isomer 11: ¹H NMR (600 MHz, CDCl₃) δ 7.05 (d, J=8.4 Hz, 1H), 6.49 (dd, J=2.4, 8.4 Hz, 1H), 6.37 (d, J=2.4 Hz, 1H), 5.14 (t, J=7.2 Hz, 1H), 4.93 (bs, 1H), 3.77 (s, 3H), 2.92 (sixt, J=7.2 Hz, 1H), 1.90-1.98 (m, 2H), 1.70 (s, 3H), 1.55-1.69 (m, 2H), 1.55 (s, 3H), 1.23 (d, J=7.2 Hz, 3H). ¹³C NMR (150 MHz, CDCl₃) δ 158.6, 154.1, 132.4, 127.7, 125.5, 124.8, 106.5, 101.9, 55.5, 37.6, 31.3, 26.2, 25.9, 21.4, 17.9. HRESIMS [M−H]⁻ m/z 233.1543 (calcd for C₁₅H₂₁O₂, 233.1542). For isomer 12: ¹H NMR (600 MHz, CDCl₃) δ 6.99 (d, J=8.4 Hz, 1H), 6.39 (s, 1H), 6.38 (d, J=7.8 Hz, 1H), 5.11 (t, J=6.6 Hz, 1H), 4.81 (bs, 1H), 3.77 (s, 3H), 3.07 (sixt, J=7.2 Hz, 1H), 1.82-1.96 (m, 2H), 1.47-1.69 (m, 8H), 1.15 (d, J=6.6 Hz, 3H). ¹³C NMR (150 MHz, CDCl₃) δ 158.3, 154.5, 131.3, 128.4, 127.5, 125.1, 106.9, 99.1, 55.6, 37.5, 31.4, 26.4, 25.9, 21.4, 17.8. HRESIMS [M−H]⁻ m/z 233.1537 (calcd for C₁₅H₂₁O₂, 233.1542).

Synthesis of Curcuphenol Analogs:

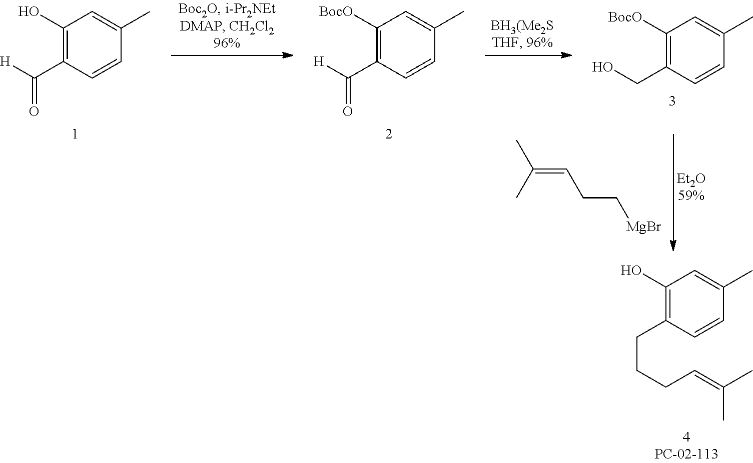

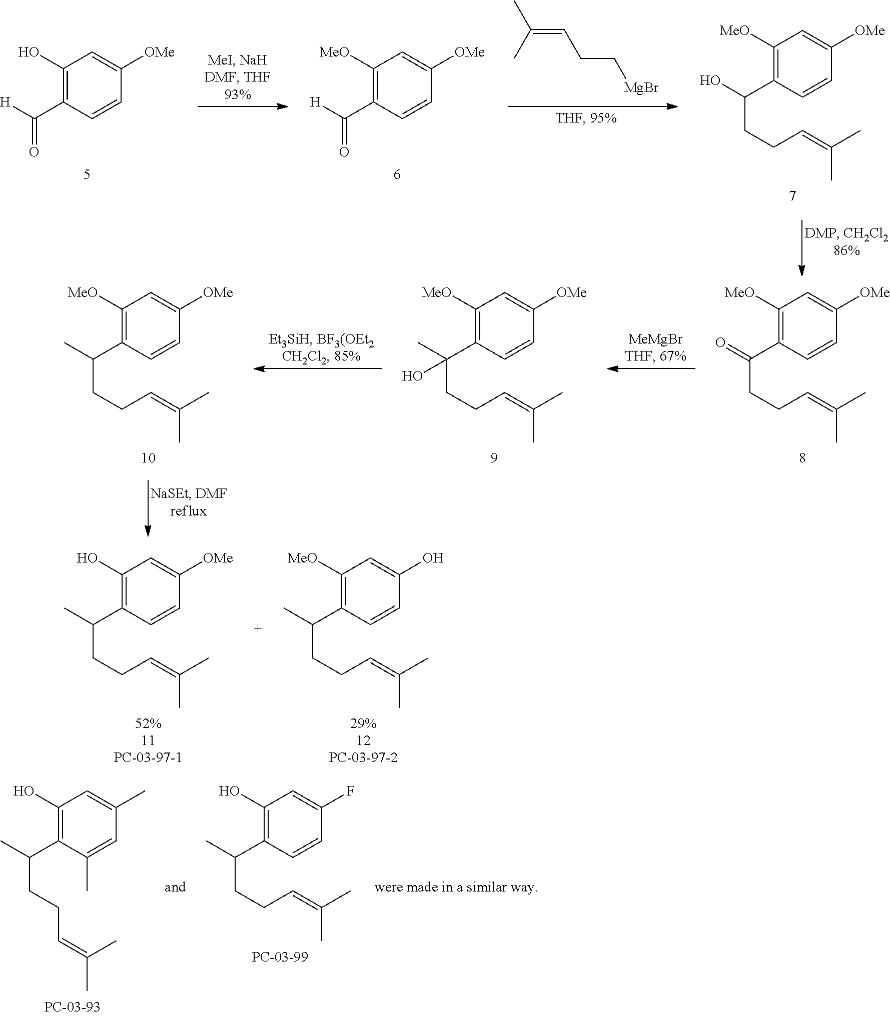

Experimental Procedure for the Synthesis of Racemic Curcuphenol

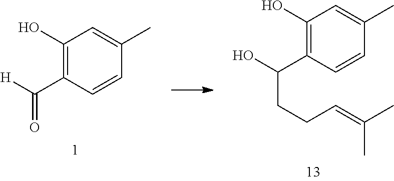

To a mixture of Mg turnings (96.7 mg, 4.03 mmol) and I₂ (tiny) in Et₂O (0.5 mL) was added several drops of a solution of 5-bromo-2-methyl-2-pentene (0.43 mL, 3.21 mmol) in Et₂O (2.5 mL). After stirring for a few minutes, the yellow solution was turned into colorless solution, then the bromide solution was added dropwise over 50 min. The reaction mixture was then stirred under reflux for 1 h. To solution of 2-hydroxy-4-methylbenzaldehyde (1) (106.5 mg, 0.76 mmol) in THF (6 mL) was added the freshly prepared Grignard reagent at room temperature. The mixture was stirred under reflux for 0.5 h and then cooled to room temperature. The reaction was quenched with saturated aqeuous NH₄Cl solution and extracted with EtOAc for three times. The combined organic extracts were washed with brine, dried over MgSO₄ and evaporated under vacuum. The residue was purified by flash chromatography (silica gel, step gradient from 5:100 Et₂O/hexanes to 10:100 Et₂O/hexanes, then 10:100 EtOAc/hexanes) to give compound 13 (170.4 mg, 100%) as a colorless oil. ¹H NMR (400 MHz, CDCl₃) δ 7.98 (s, 1H), 6.81 (d, J=7.6 Hz, 1H), 6.67 (s, 1H), 6.64 (d, J=7.6 Hz, 1H), 5.15 (t, J=7.2 Hz, 1H), 4.76-4.81 (m, 1H), 2.92 (d, J=3.2 Hz, 1H), 2.28 (s, 3H), 2.04-2.16 (m, 1H), 1.88-1.98 (m, 1H), 1.75-1.84 (m, 1H), 1.71 (s, 3H), 1.62 (s, 3H). ¹³C NMR (100 MHz, CDCl₃) δ 155.5, 139.1, 132.9, 127.2, 124.7, 123.7, 120.7, 117.9, 75.8, 37.3, 25.9, 24.6, 21.2, 18.0.

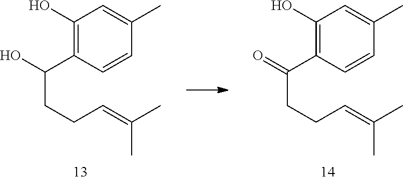 [SMILES: CC(C)=CCCC(O)c1ccc(C)cc1O ; CC(C)=CCCC(=O)c1ccc(C)cc1O]

To a solution of 13 (23.1 mg, 0.11 mmol) in CH₂Cl₂ (1 mL) was added MnO₂ (107.4 mg, 1.05 mmol) at room temperature. The mixture was stirred for 24 h, and TLC analysis showed a complete disappearance of the starting material. Then the mixture was filtered through a Celite pad and rinsed with CH₂Cl₂. The filtrate was concentrated to give a brown residue.

The residue was purified by flash chromatography (silica gel, step gradient from 0:100 Et₂O/hexanes to 2:100 Et₂O/hexanes) to give compound 14 (PC-02-116) (7.0 mg, 31%) as a colorless oil. ¹H NMR (400 MHz, CDCl₃) δ 12.38 (s, 1H), 7.63 (d, J=8.4 Hz, 1H), 6.79 (s, 1H), 6.70 (d, J=8.0 Hz, 1H), 5.13-5.18 (m, 1H), 2.98 (t, J=7.2 Hz, 2H), 2.42 (q, J=7.2 Hz, 2H), 2.35 (s, 3H), 1.70 (s, 3H), 1.64 (s, 3H). ¹³C NMR (100 MHz, CDCl₃) δ 206.0, 162.8, 148.0, 133.4, 130.1, 122.8, 120.3, 118.7, 117.4, 38.5, 25.9, 23.4, 22.1, 17.9.

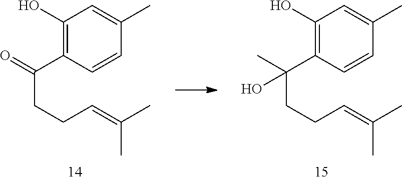 [SMILES: CC(C)=CCCC(=O)c1ccc(C)cc1O ; CC(C)=CCCC(C)(O)c1ccc(C)cc1O]

To solution of 14 (17.4 mg, 0.08 mmol) in THF (1 mL) was slowly added MeMgBr solution (0.17 mL, 3.0 M in Et₂O, 0.51 mmol) at 0° C. The mixture was stirred at 0° C. for 30 min, then the cooling bath was removed. The stirring was continued for 18 h at room temperature. Then the reaction was quenched with saturated aqeuous NH₄Cl solution and extracted with Et₂O for three times. The combined organic extracts were washed with brine, dried over MgSO₄ and evaporated under vacuum. The residue was purified by flash chromatography (silica gel, step gradient from 0:100 EtOAc/hexanes to 10:100 EtOAc/hexanes) to give compound 15 (17.0 mg, 91%) as a colorless oil. ¹H NMR (400 MHz, CDCl₃) δ 9.15 (s, 1H), 6.87 (d, J=8.0 Hz, 1H), 6.68 (s, 1H), 6.63 (d, J=8.0 Hz, 1H), 5.10-5.17 (m, 1H), 2.68 (s, 1H), 2.27 (s, 3H), 1.98-2.12 (m, 3H), 1.80-1.90 (m, 1H), 1.67 (s, 3H), 1.61 (s, 3H), 1.53 (s, 3H). ¹³C NMR (100 MHz, CDCl₃) δ 156.2, 139.0, 133.3, 126.5, 126.1, 124.0, 120.4, 118.4, 79.4, 42.3, 29.7, 25.9, 23.2, 21.1, 17.8.

Synthesis of Racemic Curcuphenol

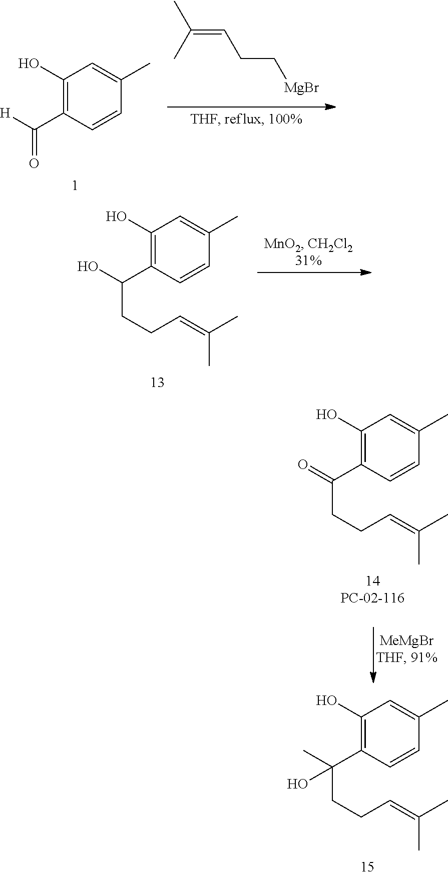

### REFERENCES

1. Lankat-Buttgereit, B. & Tampe, R. Physiol Rev 82, 187-204 (2002).
2. Gabathuler, R., et al. The Journal of experimental medicine 180, 1415-1425 (1994).
3. Jefferies, W. A., et al. The Journal of Immunology 151, 2974-2985 (1993).
4. Spies, T. et al. Nature 355, 644-646 (1992).
5. Diedrich, G., et al. J Immunol 166, 1703-1709. (2001).
6. Cabrera, T. et al. High frequency of altered HLA class I phenotypes in laryngeal carcinomas. Hum Immunol 61, 499-506 (2000).
7. Seliger, B., Cabrera, T., Garrido, F. & Ferrone, S. HLA class I antigen abnormalities and immune escape by malignant cells. Semin Cancer Biol 12, 3-13 (2002).
8. Alimonti, J. et al. TAP expression provides a general method for improving the recognition of malignant cells in vivo. Nat Biotechnol 18, 515-520. (2000).
9. Sanda, M. G. et al. Molecular characterization of defective antigen processing in human prostate cancer. J Natl Cancer Inst 87, 280-285 (1995).
10. Tanaka, K., Isselbacher, K. J., Khoury, G. & Jay, G. Reversal of oncogenesis by the expression of a major histocompatibility complex class I gene. Science 228, 26-30 (1985).
11. Nicolson, G. L. T Cancer Res 47, 1473-1487 (1987).
12. Alpan, R. S., Zhang, M. & Pardee, A. B. Cancer Res 56, 4358-4361 (1996).
13. McDermott, R. S. et al. Pathobiology 70, 324-332 (2002).
14. Kitamura, K., et al. Eur J Immunol 27, 383-388 (1997).
15. Kamarashev, J. et al. Int J Cancer 95, 23-28 (2001).
16. Chang, C. C. et al. J Biol Chem 281, 18763-18773 (2006).
17. Cabrera, C. M., L et al. Int J Cancer 113, 611-618 (2005).
18. Andratschke, M., et al Anticancer Res 23, 1467-1471 (2003).
19. Mehta, A. M., et al. Cancer Immunol Immunother 57, 197-206 (2008).
20. Zhang, H. et al. Cancer Immun 3, 2 (2003).
21. Blades, R. A. et al. Urology 46, 681-686; discussion 686-687 (1995).
22. Naoe, M. et al. BJU Int 90, 748-753 (2002).
23. Lou, Y. et al. Clinical Cancer Research 14, 1494-1501 (2008).
24. Lou, Y. et al. Vaccine 25, 2331-2339 (2007).
25. Brucet, M., et al. Genes and immunity 5, 26-35 (2004).
26. Zhang, Q. J. et al. Int J Cancer (2007).
27. Lou, Y. et al. Cancer Res 65, 7926-7933 (2005).
28. Setiadi, A. F. et al. Cancer Research 68, 9601-9607 (2008).
29. Setiadi, A. F. et al. Molecular and cellular biology 27, 7886-7894 (2007).
30. Peixoto, P. & Lansiaux, A. [Histone-deacetylases inhibitors: from TSA to SAHA]. Bull Cancer 93, 27-36 (2006).
31. Marks, P. et al. Nat Rev Cancer 1, 194-202 (2001).
32. Mukhopadhyay, N. K. et al. Ann Thorac Surg 81, 1034-1042 (2006).
33. Zhu, W. G. & Otterson, G. A. Curr Med Chem Anticancer Agents 3, 187-199 (2003).
34. Monneret, C. Eur J Med Chem 40, 1-13 (2005).
35. Cragg, G. M. L. et al. Anticancer agents from natural products, Edn. 2nd. (CRC Press, Boca Raton; 2012).
36. Newman, D. J. & Cragg, G. M. J Nat Prod 79, 629-661 (2016).
37. Newman, D. J. & Cragg, G. M. Planta Med 82, 775-789 (2016).
38. Andersen, R. J. Biochemical pharmacology 139, 3-14 (2017).
39. Brastianos, H. C. et al. J Am Chem Soc 128, 16046-16047 (2006).
40. Carlile, G. W. et al. Chemistry & biology 19, 1288-1299 (2012).
41. Karjala, G., et al. Cancer Res 65, 3040-3043 (2005).
42. Loganzo, F. et al. Cancer Res 63, 1838-1845 (2003).
43. Ong, C. J. et al. Blood 110, 1942-1949 (2007).
44. Setiadi, A. F. et al. Cancer Research 65, 7485-7492 (2005).
45. Lai, P. K. & Roy, J. A Curr Med Chem 11, 1451-1460 (2004).
46. Thompson, L. U., et al. Carcinogenesis 17, 1373-1376 (1996).
47. Abdullaev, F. I. Exp Biol Med (Maywood) 227, 20-25 (2002).
48. Busquets, S. et al. Cancer Lett 167, 33-38 (2001).
49. Greenwell, M. & Rahman, P. K. Int J Pharm Sci Res 6, 4103-4112 (2015).
50. Simmons, T. L., et al. Mol Cancer Ther 4, 333-342 (2005).
51. Gomes, N. G., et al. Mar Drugs 14 (2016).
52. Chmielewski, M., et al. Front Immunol 4, 371 (2013).
53. Seliger, B. HLA 88, 213-220 (2016).
54. Khalil, D. N., et al. Nat Rev Clin Oncol 13, 394 (2016).
55. Rodrigo, G. et al. Fitoterapia 81, 762-766 (2010).
56. Gaspar, H., et al. Marine Drugs 2, 8-13 (2004).
57. El Sayed, K. A. et al. J Nat Prod 65, 1547-1553 (2002).
58. Behery, F. A., et al. Med chem comm 3, 1309-1315 (2012).
59. Fusetani, N., et al. Experientia 43, 1234-1235 (1987).
60. Wright, A. E., et al. J Nat Prod 50, 976-978 (1987).
61. Gaspar, H., et al. Nat Prod Commun 3, 1457-1464 (2008).
62. Cichewicz, R. H. et al. Bioorgan Med Chem 13, 5600-5612 (2005).
63. Beher, D. et al. Chemical Biology & Drug Design 74, 619-624 (2009).
64. Chakrabarti, A. et al. Trends Pharmacol Sci 36, 481-492 (2015).
65. Gao, J., et al. Biochem Biophys Res Commun 379, 1-5 (2009).
66. Wu, J. et al. Digestive Diseases & Sciences 58, 3545-3553 (2013).
67. Vannini, A. et al. Proc Natl Acad Sci USA 101, 15064-15069 (2004).
68. Yao, H. & Rahman, I. Curr Opin Pharmacol 9, 375-383 (2009).
69. Osada, H. et al. Int J Cancer 112, 26-32 (2004).
70. Urbich, C. et al. Blood 113, 5669-5679 (2009).
71. Song, C., et al. J Biol Chem 288, 28021-28033 (2013).
72. Jin, Z. et al. Int J Clin Exp Pathol 7, 5872-5879 (2014).
73. Powers, J. et al. Methods Mol Biol 1436, 129-145 (2016).
74. CANADIAN CANCER SOCIETY, NATIONAL CANCER INSTITUTE OF CANADA, STATISTICS CANADA, P., TERRITORIAL CANCER REGISTRIES & CANADA, P.H.A.O. Canadian Cancer Statistics 2007. (2007).
75. Lee, H. M., et al. Cancer Res 60, 1927-1933. (2000).
76. Smahel, M. et al. Vaccine 21, 1125-1136 (2003).
77. Saranchova, I. et al. Scientific reports 6, 30555 (2016).
78. Zhang, J. H., et al. J Biomol Screen 4, 67-73 (1999).

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A compound which modulates expression of MHC-1 and/or TAP-1, in eukaryotic cells.

2. The compound of claim 1, wherein said compound has the structure: 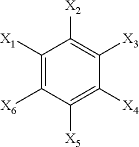
where:
X₁ is H, R, OH, OR, SH, SR, F, Cl, Br, I, OCOR, NH₂, RNH, R₂NH, NHCOR, OSO₃H, OP(OH)₃
X₂ is R₁
X₃ is H, R, OH, OR, SH, SR, F, Cl, Br, I, OCOR, NH₂, RNH, R₂NH, NHCOR, OSO₃H, OP(OH)₃
X₄ and X₆ are independently H, R, OH, OR, SH, SR, F, Cl, Br, I, OCOR, NH₂, RNH, R₂NH, NHCOR, OSO₃H, OP(OH)₃
X₅ is R₂
R is a linear, branched, or cyclic, saturated or unsaturated, one to thirty carbon alkyl group that may be substituted with one or more of OH, OR, SH, SR, ═O, F, Cl, Br, I, OCOR, NH₂, RNH, R₂NH, NHCOR, OSO₃H, OP(OH)₃, and where individual carbon atoms may be replaced by O, N, or S atoms.
R₁ is a linear, branched, or cyclic, saturated, unsaturated or aromatic, one to thirty carbon alkyl group that may be substituted with one or more of OH, OR, SH, SR, ═O, F, Cl, Br, I, OCOR, NH₂, RNH, R₂NH, NHCOR, OSO₃H, OP(OH)₃, and where individual carbon atoms may be replaced by O, N, or S atoms.
R₂ is a linear, branched, or cyclic, saturated, unsaturated, or aromatic one to twenty carbon alkyl group that may be substituted with one or more of OH, OR, SH, SR, ═O, F, Cl, Br, I, OCOR, NH₂, RNH, R₂NH, NHCOR, OSO₃H, OP(OH)₃, and where individual carbon atoms may be replaced by O, N, or S atoms.

3. The compound of claim 1, wherein said compound modulates HDAC activity as compared to activity untreated control cells.

4. The compound of claim 3, wherein said compound inhibits HDAC8 activity and upregulates HDAC5 and HDAC10.

5. The compound of claim 2, wherein
X₁ is OH or OR
X₂ is one of the following: 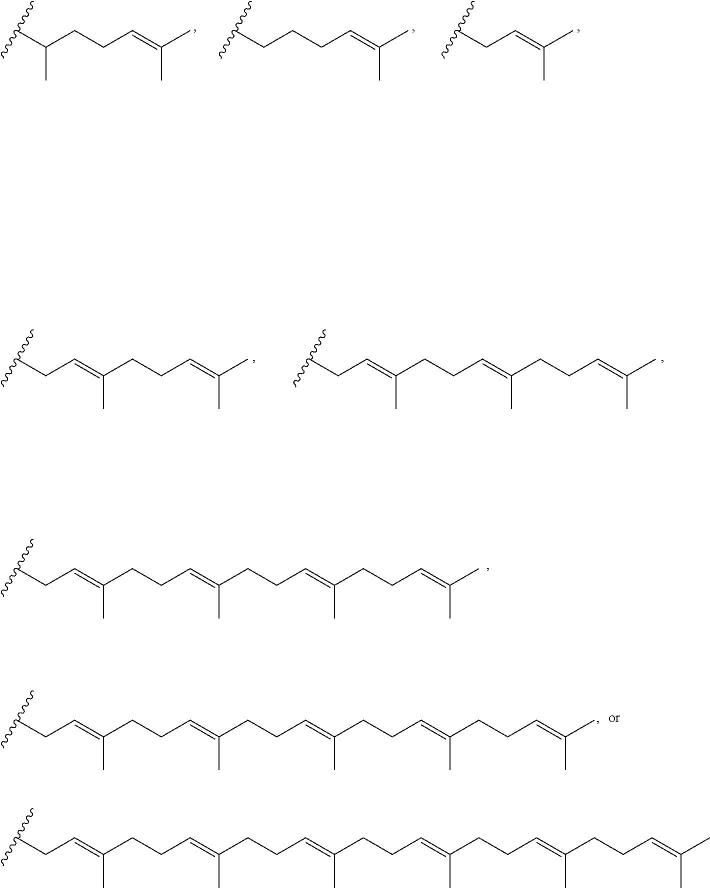
X₃ is H, OH, or OR
X₄ and Xₛ is H
X₅ is OH, OR, or methyl, ethyl, n-propyl, n-butyl, n-pentyl, n-hexyl or any seven to twenty carbon linear saturated n-alkyl

6. A compound having the structure:

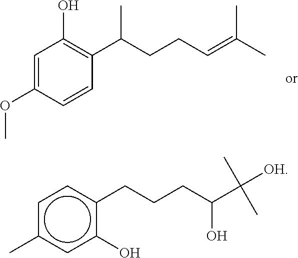

7. The compound of claim 1, wherein said compound is a terpene.

8. The compound of claim 1, wherein said compound is a cannabinoid.

9. The compound of claim 1, wherein said compound is a curcuphenol compound.

10. The compound of claim 9, wherein said curcuphenol compound is water soluble

11. A method of augmenting an immune response involving MHC-1 CTL comprising administering one or more compounds of claim 1 alone or in combination with one or more other therapeutic agents.

12. A method of treating cancer comprising administering one or more compounds of claim 1 alone or in combination with one or more other therapeutic agents.

13. A method of modulating histone acetylation comprising administering one or more compounds of claim 1 alone or in combination with one or more other therapeutic agents.

14. A method of treating a disease associated with histone acetylation abnormalities comprising administering one or more compounds of claim 1 alone or in combination with one or more other therapeutic agents.

15. The method of claim 14, wherein the disease is selected from cancer, a mood disorder or epilepsy.

16. A method of augmenting an immune response, improving general health, improving longevity and/or reducing nausea comprising administering one or more compounds of claim 1 alone or in combination with one or more other therapeutic agents.

17. A composition comprising one or more compounds of claim 1 alone or in combination with one or more other therapeutic agents and a carrier.

18. The composition of claim 17, wherein said compound has the structure:

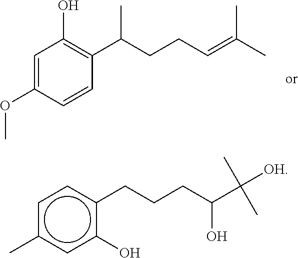

19. A natural product comprising one or more compounds of claim 1.

20. The natural product of claim 19, wherein said product comprises an extract or resin.
